(19) United States
(12) Patent Application Publication
(10) Pub. No.: US 2003/0065157 A1
(43) Pub. Date: Apr. 3, 2003

(54) Genes expressed in lung cancer

(76) Inventor: Amy W. Lasek, Oakland, CA (US)

Correspondence Address: INCYTE GENOMICS, INC., 3160 PORTER DRIVE, PALO ALTO, CA, 94304, US

(21) Appl. No.: 10/116,802

(22) Filed: Apr. 4, 2002

Related U.S. Application Data
(60) Provisional application No. 60/281,593, filed on Apr. 4, 2001.

(51) Int. Cl. C07H 21/02; C07H 21/04

(52) U.S. Cl. 536/23.1

(57) ABSTRACT

The present invention relates to a combination comprising a plurality of cDNAs which are differentially expressed in a respiratory disorder and which may be used in their entirety or in part to diagnose, to stage, to treat, or to monitor the treatment of a subject with a respiratory disorder.

This application claims benefit of provisional application 60/281,593, filed 4 Apr. 2001.

### FIELD OF THE INVENTION

The present invention relates to a combination comprising a plurality of cDNAs which are differentially expressed in lung cancer and which may be used entirely or in part to diagnose, to stage, to treat, or to monitor the progression or treatment of lung cancer.

### BACKGROUND OF THE INVENTION

Lung cancer is the leading cause of cancer death in the United States affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups and four histopathological classes. The Non Small Cell Lung Carcinoma (NSCLC) group accounts for about 70% of all lung cancer cases and includes adenocarcinomas, squamous cell carcinomas, and large cell carcinomas. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium that leads to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the roles of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999, Genomics 51:5-14) identified five genes differentially expressed in lung cancer cell lines compared to normal bronchial epithelial cells. Among the known genes, pulmonary surfactant apoprotein A and alpha 2 macroglobulin were down-regulated, and nm23H1 was upregulated. Petersen et al. (2000, Int J Cancer 86:512-517) used suppression subtractive hybridization to identify 552 clones differentially expressed in lung tumor derived cell lines; 205 of these clones represented known genes. Among the known genes, thrombospondin-1, fibronectin, intercellular adhesion molecule 1, and cytokeratins 6 and 18 had been observed previously to be differentially expressed in lung cancers. Wang et al. (2000, Oncogene 19:1519-1528) used a combination of microarray analysis and subtractive hybridization to identify 17 genes differentially overexpresssed in squamous cell carcinoma compared with normal lung epithelium. Keratin isoform 6, KOC, SPRC, IGFb2, connexin 26, plakofillin 1 and cytokeratin 13 were identified among the known genes..

Array technologies provide a simple way to explore the expression profile of a large number of related or unrelated genes. When an expression profile is examined, arrays provide a platform for examining which genes are tissue-specific, carrying out housekeeping functions, parts of a signaling cascade, or specifically related to a particular genetic predisposition, condition, disease, or disorder. The potential application of gene expression profiling is particularly relevant to improving diagnosis, prognosis, and treatment of disease. For example, both the sequences and the amount of expression can be compared between tissues from subjects with lung cancer and cytologically normal lung tissue.

The present invention provides for a combination comprising a plurality of cDNAs for use in detecting changes in expression of genes encoding proteins that are associated with lung cancer. The present invention satisfies a need in the art by providing a set of cDNAs that represent genes that are differentially expressed in lung cancers, particularly adenocarcinoma and squamous cell carcinoma, and can be used entirely or in part to diagnose, to stage, to treat, or to monitor the progression or treatment of a subject with lung cancer.

### SUMMARY

The present invention provides a combination comprising a plurality of cDNAs wherein the cDNAs are SEQ ID NOs:1-519 as presented in the Sequence Listing that are differentially expressed in lung cancers and the complements of SEQ ID NOs:1-519. The invention also provides a combination comprising a plurality of cDNAs wherein the cDNAs are SEQ ID NOs:1-12 that are differentially expressed at least 2.5-fold in lung cancers and the complements of SEQ ID NOs:1-12. The invention further provides a combination comprising a plurality of cDNAs wherein the cDNAs are SEQ ID NOs:13-83 that are differentially expressed at least 2.0-fold in lung cancers and the complements of SEQ ID NOs:13-83. The invention still further provides a combination comprising a plurality of cDNAs wherein the cDNAs are SEQ ID NOs:98, 132, 135, 140, 145, 152, 174, 210, 223, 242, 246, 278, 304, 340, and 490 that are differentially expressed in squamous cell carcinomas thereby distinguishing squamous cell carcinoma from adenocarcinoma and the complements of SEQ ID NOs:98, 132, 135, 140, 145, 152, 174, 210, 223, 242, 246, 278, 304, 340, and 490. In one aspect, a combination is useful to diagnose a respiratory disorder such as lung cancer. In another aspect, a combination is immobilized on a substrate.

The invention also provides a high throughput method to detect differential expression of one or more of the cDNAs of the combination. The method comprises hybridizing the substrate comprising the combination with the nucleic acids of a sample, thereby forming one or more hybridization complexes, detecting the hybridization complexes, and comparing the hybridization complexes with those of a standard, wherein differences in the size and signal intensity of each hybridization complex indicates differential expression of nucleic acids in the sample. In one aspect, the nucleic acids of the sample are amplified prior to hybridization. In another aspect, the sample is from a subject with lung cancer and differential expression identifies the type of lung cancer or stages the lung cancer.

The invention provides a high throughput method of screening a library or a plurality of molecules or compounds to identify a ligand. The method comprises combining the substrate comprising the combination with a library or a plurality of molecules or compounds under conditions to allow specific binding and detecting specific binding, thereby identifying a ligand. The library or plurality of molecules or compounds are selected from DNA molecules, enhancers, mimetics, peptide nucleic acids, proteins, repressors, regulatory proteins, RNA molecules, and transcription factors. The invention also provides a method for purifying a ligand, the method comprising combining a cDNA of the invention with a sample under conditions which allow specific binding, recovering the bound cDNA, and separating the cDNA from the ligand, thereby obtaining purified ligand.

The invention provides an isolated cDNA comprising a nucleic acid selected from SEQ ID NOs:12, 45, 51, 56, 64, 70, 72, 75-83, 344, 346, 375, 376, 377, 402, 406, 407, 412, 419, and 431 as presented in the Sequence Listing. The invention also provides a vector comprising the cDNA, a host cell comprising the vector, and a method for producing a protein comprising culturing the host cell under conditions for the expression of a protein and recovering the protein from the host cell culture.

The present invention provides a purified protein encoded and produced by a cDNA of the invention. The invention also provides a high-throughput method for using a protein to screen a library or a plurality of molecules or compounds to identify a ligand. The method comprises combining the protein or a portion thereof with the library or plurality of molecules or compounds under conditions to allow specific binding and detecting specific binding, thereby identifying a ligand which specifically binds the protein. The library or plurality of molecules or compounds is selected from agonists, antagonists, antibodies, DNA molecules, small molecule drugs, immunoglobulins, inhibitors, mimetics, peptide nucleic acids, peptides, pharmaceutical agents, proteins, RNA molecules, and ribozymes. In one aspect, an antibody which specifically binds a protein of the invention is selected from a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein. The invention further provides a method for using a protein to purify a ligand. The method comprises combining the protein or a portion thereof with a sample under conditions to allow specific binding, recovering the bound protein, and separating the protein from the ligand, thereby obtaining purified ligand. The invention still further provides method for using the protein to produce a polyclonal or a monoclonal antibody. The method for producing a polyclonal antibody comprises immunizing a animal with a protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibody comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating a monoclonal antibody from culture. The method comprises immunizing an animal with the protein or an antigenic determinant thereof under conditions to elicit an antibody response, isolating animal antibodies, and screening the isolated antibodies with the protein to identify an antibody which specifically binds the protein. The invention yet still further provides a method for using the protein to purify antibodies which bind specifically to the protein.

The invention provides a purified antibody which specifically binds a protein of the invention. The invention also provides a method of using an antibody to detect the expression of a protein in a sample, the method comprising contacting the antibody with a sample under conditions for the formation of an antibody:protein complex and detecting complex formation wherein the formation of the complex indicates the expression of the protein in the sample. In one aspect, complex formation is compared to standards and is diagnostic of a respiratory disorder such as lung cancer. The invention further provides using an antibody to immunopurify a protein comprising combining the antibody with a sample under conditions to allow formation of an antibody:protein complex, and separating the antibody from the protein, thereby obtaining purified protein.

The invention provides a composition comprising a cDNA, a protein, an antibody, or a ligand which has agonistic or antagonistic activity.

### DESCRIPTION OF THE COMPACT DISC-RECORDABLE (CD-R)

CD-R 1 is labeled: “PA-0045 US, Copy 1,” was created on Apr. 4, 2002 and contains: the Sequence Listing formatted in plain ASCII text. The file for the Sequence Listing is entitled pa0045.txt, created on Apr. 4, 2002 and is 1.294 KB in size.

CD-R 2 is an exact copy of CD-R 1. CD-R 2 is labeled: “PA-0045 US, Copy 2,” and was created on Apr. 4, 2002.

The CD-R labeled as: “PA-0045 US, CRF,” contains the Sequence Listing formatted in plain ASCII text. The file for the Sequence Listing is entitled pa0045us.txt, was created on Apr. 4, 2002 and is 1.294 KB in size.

The content of the Sequence Listing named above and as described below, submitted in duplicate on two (2) CD-Rs (labeled “PA-0045 US, Copy 1” and “PA-0045 US, Copy 2”), and the CRF (labeled “PA-0045 US, CRF”) containing the Sequence Listing, are incorporated by reference herein, in their entirety.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of cDNAs obtained by sequencing and extending clone inserts. Each sequence is identified by a sequence identification number (SEQ ID NO) and by the Incyte template number (INCYTE ID) from which it was obtained.

Table 1 lists the differential expression of clones representing cDNAs of the present invention on microarrays. Columns 1, 2, and 3 show the SEQ ID NO, TEMPLATE ID, and CLONE ID, respectively. Columns 4 through 10 show the differential expression of each cDNA in a lung tumor sample relative to non-involved lung tissue from the same patient. Differential expression values are in log base 2. Negative values indicate downregulation of the cDNA in lung tumor.

Table 2 lists the functional annotation of the cDNAs of the present invention. Columns 1, 2, and 3 show the SEQ ID NO, TEMPLATE ID, and CLONE ID, respectively. Columns 3, 4, and 5 show the GenBank hit (GENBANK ID), probability score (E-VALUE), and functional annotation, respectively, as determined by BLAST analysis (version 2.0 using default parameters; Altschul et al. (1997) Nucleic Acids Res 25:3389-3402; Altschul (1993) J Mol Evol 36: 290-300; and Altschul e al. (1990) J Mol Biol 215:403-410) of the cDNA against GenBank (release 121; National Center for Biotechnology Information (NCBI), Bethesda Md.).

Table 3 shows the region of each cDNA encompassed by the clone present on a microarray and identified as differentially expressed. Columns 1 and 2 show the SEQ ID NO and TEMPLATE ID, respectively. Column 3 shows the CLONE ID and columns 4 and 5 show the first residue (START) and last residue (STOP) encompassed by the clone on the template.

### DESCRIPTION OF THE INVENTION

### Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

A “combination” refers to at least two cDNAs and up to about 1038 cDNAs selected from SEQ ID NOs:1-519 and the complements thereof.

“cDNA” refers to an isolated polynucleotide, nucleic acid, or a fragment thereof, that contains from about 400 to about 12,000 nucleotides. It may have originated recombinantly or synthetically, may be double-stranded or single-stranded, represents coding and noncoding 3′ or 5′ sequence, generally lacks introns and may be purified or combined with carbohydrate, lipids, protein or inorganic elements or substances.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul, supra; Altschul (1990) supra) and BLAST2 (Altschul (1997) supra) which provide identity within the conserved region. Brenner et al. (1998, Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a vector or a labeling moiety; a purified protein and a pharmaceutical carrier or a heterologous, labeling, or purification moiety; an antibody and a labeling moiety or pharmaceutical agent; and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased, upregulated or present, or decreased, downregulated or absent, gene expression as detected by the absence, presence, or at least two-fold change in the amount of transcribed messenger RNA or translated protein in a sample.

“Disorder” refers to conditions, diseases or syndromes of the respiratory system including, but not limited to, lung cancer, particularly adenocarcinomas and squamous cell carcinomas; chronic obstructive pulmonary disease, emphysema, or asthma.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses PAGE, ELISA, FACS, or arrays and labeling moieties or antibodies to detect expression in a sample. The nucleic acids, proteins, or antibodies may be used in solution or attached to a substrate, and their detection is based on methods well known in the art.

“Fragment” refers to a chain of consecutive nucleotides from about 60 to about 5000 base pairs in length. Fragments may be used in PCR, hybridization or array technologies to identify related nucleic acids and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997) supra). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Isolated” or “purified” refers to any molecule or compound that is separated from its natural environment and is from about 60% free to about 90% free from other components with which it is naturally associated.

“Labeling moiety” refers to any reporter molecule whether a visible or radioactive label, stain or dye that can be attached to or incorporated into a cDNA or protein. Visible labels and dyes include but are not limited to anthocyanins, 8 glucuronidase, BIODIPY, Coomassie blue, Cy3 and Cy5, digoxigenin, FITC, green fluorescent protein, luciferase, spyro red, silver, and the like. Radioactive markers include radioactive forms of hydrogen, iodine, phosphorous, sulfur, and the like.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

“Oligonucleotide” refers a single stranded molecule from about 18 to about 60 nucleotides in length which may be used in hybridization or amplification technologies or in regulation of replication, transcription or translation. Equivalent terms are amplimer, primer, and oligomer.

“Portion” refers to any part of a protein used for any purpose which retains at least one biological or antigenic characteristic of a native protein, but especially, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid molecule in a sample. Where targets are single stranded, probes are complementary single strands. Probes can be labeled for use in hybridization reactions including Southern, northern, in situ, dot blot, array, and like technologies or in screening assays.

“Protein” refers to a polypeptide or any portion thereof. An “oligopeptide” is an amino acid sequence from about five residues to about 15 residues that is used as part of a fusion protein to produce an antibody.

“Sample” is used in its broadest sense as containing nucleic acids, proteins, antibodies, and the like. A sample may comprise a bodily fluid such as ascites, blood, lymph, semen, sputum, urine and the like;; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, tissue biopsy or tissue print; buccal cells, skin, a hair or its follicle; and the like.

“Specific binding” refers to a special and precise interaction between two molecules which is dependent upon their structure, particularly their molecular side groups. Examples include the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a cDNA or a protein encoded by the cDNA. Splice variants may be determined by BLAST score, wherein the score is at least 100, and most preferably at least 400. Allelic variants have high percent identity to the cDNAs of the invention and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

### The Invention

The present invention provides a combination comprising a plurality of cDNAs, wherein the cDNAs are SEQ ID NOs:1-519 of the Sequence Listing, are differentially expressed in lung tissues, and can be used to detect changes in expression associated with respiratory disorders, particularly adenocarcinomas and squamous cell carcinomas of the lung, chronic obstructive pulmonary disease, emphysema, and asthma. The invention also provides a combination comprising a plurality of cDNAs wherein the cDNAs are SEQ ID NOs:1-12 that are differentially expressed at least 2.5-fold in lung cancers and the complements of SEQ ID NOs:1-12. The invention further provides a combination comprising a plurality of cDNAs wherein the cDNAs are SEQ ID NOs:13-83 that are differentially expressed at least 2.0-fold in lung cancers and the complements of SEQ ID NOs:13-83. The invention still further provides a combination comprising a plurality of cDNAs wherein the cDNAs are SEQ ID NOs:98, 132, 135, 140, 145, 152, 174, 210, 223, 242, 246, 278, 304, 340, and 490 that are differentially expressed in squamous cell carcinoma thereby distinguishing squamous cell carcinoma from adenocarcinoma and the complements of SEQ ID NOs:98, 132, 135, 140, 145, 152, 174, 210, 223, 242, 246, 278, 304, 340, and 490.

Table 1 shows those cDNAs having the nucleic acid sequences of SEQ ID NOs:1-83 that are regulated more than 2.5-fold in all tumors (log₂>|1.32|). Table 1 also shows the cDNAs having the nucleic acid sequences of SEQ ID NOs:84-519 which are regulated more than two-fold in a majority of tumors. These combinations are useful in the diagnosis of respiratory disorders, especially when immobilized on a substrate. The cDNAs comprising the nucleic acids of SEQ ID NOs:12, 45, 51, 56, 64, 70, 72, 75-83, 344, 346, 375, 376, 377, 402, 406, 407, 412, 419, and 431 are novel cDNAs and are known only by their differential expression in lung tumor tissue. The usefulness of the novel cDNAs exists in their immediate value as diagnostics for respiratory disorders such as lung cancer.

Table 1 lists each clone representing cDNAs of the present invention that showed differential expression on a microarray. Columns 1, 2, and 3 show the SEQ ID NO, Template ID, and Clone ID, respectively. Columns 4 through 10 show the differential expression of each cDNA in a lung tumor sample relative to non-involved lung tissue from the same patient. Differential expression values are in log base 2. Negative values indicate upregulation of the cDNA in lung tumor. Column 11 shows a two-tailed t-test assuming unequal variance comparing values obtained from squamous cell carcinomas to values obtained from adenocarcinomas. T-test probability scores of less than 0.05 identify genes that show significantly different expression in one carcinoma type relative to the other. The cDNAs of SEQ ID NOs:98, 132, 135, 140, 145, 152, 174, 210, 223, 242, 246, 278, 304, 340, and 490 are of particular importance to distinguish squamous cell carcinomas from adenocarcinomas. Table 2 lists the functional annotation of the cDNAs of the present invention. Columns 1, 2, and 3 show the SEQ ID NO, Template ID, and Clone ID, respectively. Columns 3, 4, and 5 show the GenBank ID, probability score (E-Value), and functional annotation, respectively, as determined by BLAST2 (Altschul_(1997) supra) of the cDNA against GenBank (release 121; NCBI). Table 3 shows the region of each cDNA encompassed by the clone; columns 1, 2, and 3 show the SEQ ID NO, Template ID, and Clone ID respectively; and columns 4 and 5 show the first residue and last residue encompassed by the clone on the template.

The cDNAs of the invention define a differential expression pattern against which to compare the expression pattern of biopsied and/or in vitro treated lung tissues. The combination may be arranged on a substrate and hybridized with tissues from subjects with other diagnosed respiratory disorders. This will provide information useful for distinguishing among different disorders, or stages of those disorders, and identifying those sequences of highest diagnostic and potential therapeutic value for each disorder. In another embodiment, an additional set of useful cDNAs, such as cDNAs encoding signaling molecules, are arranged on the substrate with the combination. Such combinations may be useful in the elucidation of pathways which are affected in a particular disorder or to identify new, coexpressed, candidate, therapeutic molecules.

In another embodiment, the combination can be used for large scale genetic or gene expression analysis of a number of nucleic acids. These samples are prepared by methods well known in the art and are derived from mammalian cells or tissues which are in a certain stage of development; have been treated with a known molecule or compound, such as a cytokine, growth factor, drug, or the like; or have been extracted or biopsied from a mammal with a known or unknown condition, disorder, or disease before or after treatment. The sample nucleic acids are hybridized to the combination for the purpose of defining a novel gene profile associated with that developmental stage, treatment, or disorder.

### cDNAs and Their Uses

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7) 215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2, 6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate. Synthesis on the surface of a substrate may be accomplished using a chemical coupling procedure and a piezoelectric printing apparatus as described by Baldeschweiler et al. (PCT publication WO95/25 1116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

The cDNAs may be used for a variety of purposes. For example, the combination of the invention may be used on an array. The array, in turn, can be used in high-throughput methods for detecting a related polynucleotide in a sample, screening a plurality of molecules or compounds to identify a ligand, diagnosing a respiratory disorder such as chronic obstructive pulmonary disease or lung cancer, or inhibiting or inactivating a therapeutically relevant gene related to the cDNA.

When the cDNAs of the invention are employed on a microarray, the cDNAs are arranged in an ordered fashion so that each cDNA is present at a specified location. Because the cDNAs are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

### Hybridization

The cDNAs or fragments or complements thereof may be used in various hybridization technologies. The cDNAs may be labeled using a variety of reporter molecules by either PCR, recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Pharmacia Biotech (APB), Piscataway NJ), fluorescent (Qiagen-Operon, Alameda Calif.), and chemiluminescent labeling (Promega, Madison Wis.) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide, a fragment of the cDNA, may be used to detect a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5× saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1% SDS at either 45° C. (medium stringency) or 65° C.-68° C. (high stringency). At high stringency, hybridization complexes will remain stable only where the nucleic acids are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York NY, Units 2.8-2.11, 3.18-3.19 and 4-6-4.9).

Dot-blot, slot-blot, low density and high density arrays are prepared and analyzed using methods known in the art. cDNAs from about 18 consecutive nucleotides to about 6000 consecutive nucleotides in length are contemplated by the invention and used in array technologies. The number of cDNAs on a substrate is at least about five and can be up to about 100,000. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; W095/11995; WO95/35505; U.S. Pat. No. 5,605,662; and U.S. Pat. No. 5,958,342.)

### Screening and Purification Assays

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of the polynucleotide in the biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

The cDNA may be used to purify a ligand from a sample. A method for using a cDNA to purify a ligand would involve combining the cDNA or a fragment thereof with a sample under conditions to allow specific binding, recovering the bound cDNA, and using an appropriate agent to separate the cDNA from the purified ligand.

### Protein Production and Uses

The cDNAs or fragment thereof may be used to produce purified proteins using recombinant DNA technologies described herein and taught in Ausubel (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.) and algorithms included in RasMol software (University of Massachusetts, Amherst Mass.) may be used to determine reading frame and which and how many amino acid residues in a particular portion of the protein may be substituted, inserted, or deleted without abolishing biological or immunological activity.

### Expression of Encoded Proteins

Expression of a particular cDNA may be accomplished by cloning the cDNA into a vector and transforming this vector into a host cell. The cloning vector used for the construction of cDNA libraries in the LIFESEQ databases (Incyte Genomics, Palo Alto Calif.) may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside (IPTG) using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters includes SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the 431A peptide synthesizer (Applied Biosystems (ABI), Foster City Calif.). Proteins produced by any of the above methods may be used as pharmaceutical compositions to treat disorders associated with null or inadequate expression of the genomic sequence.

### Screening and Purification Assays

A protein or a portion thereof encoded by the cDNA may be used to screen a library or a plurality of molecules or compounds for a ligand with specific binding affinity or to purify a molecule or compound from a sample. The protein or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against a library or a plurality of ligands and the specificity of binding or formation of complexes between the expressed protein and the ligand may be measured. The ligands can be agonists, antagonists, antibodies, DNA molecules, enhancers, small drug molecules, immunoglobulins, inhibitors, mimetics, peptide nucleic acid molecules, peptides, pharmaceutical agents, proteins, and regulatory proteins, repressors, RNA molecules, ribozymes, and transcription factors, or any other test molecule or compound that specifically binds the protein. An exemplary assay involves combining the mammalian protein or a portion thereof with the molecules or compounds under conditions that allow specific binding and detecting the bound protein to identify at least one ligand that specifically binds the protein.

This invention also contemplates the use of competitive drug screening assays in which neutralizing antibodies capable of binding the protein specifically compete with a test compound capable of binding to the protein or oligopeptide or fragment thereof. One method for high throughput screening using very small assay volumes and very small amounts of test compound is described in U.S. Pat. No. 5,876,946. Molecules or compounds identified by screening may be used in a model system to evaluate their toxicity, diagnostic, or therapeutic potential.

The protein may be used to purify a ligand from a sample. A method for using a protein to purify a ligand would involve combining the protein or a portion thereof with a sample under conditions to allow specific binding, recovering the bound protein, and using an appropriate chaotropic agent to separate the protein from the purified ligand.

### Antibody Production and Use

A protein encoded by a cDNA of the invention may be used to produce specific antibodies. Antibodies may be produced using an protein, polypeptide, peptide or oligopeptide with inherent antigenicity. Methods for producing antibodies include:1) injecting an animal, usually goats, rabbits, or mice, with the protein, or an epitope or oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

### Antibody Arrays

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt et al. (2000) Nature Biotechnol 18:989-94).

### Labeling of Molecules for Assay

A wide variety of reporter molecules and conjugation techniques are known by those skilled in the art and can be used with cDNAs, polynucleotides, proteins, peptides or antibodies in screening, purification, and diagnostic assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

### DIAGNOSTICS

The cDNAs, or fragments thereof, may be used to detect and quantify differential gene expression; absence, presence, or excess expression of mRNAs; or to monitor mRNA levels during therapeutic intervention. Disorders associated with altered expression include chronic obstructive pulmonary disease, lung cancer, emphysema and asthma. These cDNAs can also be utilized as markers of treatment efficacy against the disorders noted above and other disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

### Gene Expression Profiles

A gene expression profile comprises a plurality of cDNAs and a plurality of detectable hybridization complexes, wherein each complex is formed by hybridization of one or more probes to one or more complementary nucleic acids in a sample. The cDNAs of the invention are used as elements on an array to analyze gene expression profiles. In one embodiment, the array is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

Experimentally, expression profiles can also be evaluated by methods including, but not limited to, differential display by spatial immobilization, gel electrophoresis, genome mismatch scanning, representational discriminate analysis, clustering, and transcript imaging, and protein or antibody arrays. Expression profiles produced by these methods may be used alone or in combination. The correspondence between mRNA and protein expression has been discussed by Zweiger (2001, Transducing the Genome. McGraw-Hill, San Francisco, Calif.) and Glavas et al. (2001, T cell activation upregulates cyclic nucleotide phosphodiesterases 8A1 and 7A3, Proc Natl Acad Sci 98:6319-6342) among others.

In another embodiment, animal models which mimic a human disease can be used to characterize expression profiles associated with a particular condition, disorder or disease or its treatment. Novel treatment regimens may be tested in these animal models using arrays to establish and then follow expression profiles over time. In addition, arrays can be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

### Assays Using Antibodies

Antibodies directed against epitopes on a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, fluorescent activated cell sorting (FACS), and arrays. Such immunoassays typically involve the formation of complexes between the protein and its specific antibody and the measurement of such complexes.

### THERAPEUTICS

The cDNAs and fragments thereof can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overexpression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997)Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These sequences can be produced biologically by transforming an appropriate host cell with a vector containing the sequence of interest.

Molecules which regulate the activity of the cDNA or encoded protein are useful as therapeutics for respiratory disorders including chronic obstructive pulmonary disease, lung cancer, emphysema and asthma. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

### Transgenic Animal Models

Transgenic rodents that overexpress or underexpress a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

### Embryonic Stem Cells

Embryonic (ES) stem cells isolated from rodent embryos retain the potential to form embryonic tissues. When ES cells such as the mouse 129/SvJ cell line are placed in a blastocyst from the C57BL/6 mouse strain, they resume normal development and contribute to tissues of the live-born animal. ES cells are preferred for use in the creation of experimental knockout and knockin animals. The method for this process is well known in the art and the steps are:the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

### Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

### Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

As described herein, the uses of the cDNAs, provided in the Sequence Listing of this application, and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining, assembling or expressing cDNAs that will be known to those skilled in the art. It is also to be understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

### I Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Invitrogen). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla Calif.) was provided with RNA and constructed the cDNA libraries. Otherwise, cDNA was synthesized and cDNA libraries were constructed with the UNIZAP vector system (Stratagene) or SUPERSCRIPT plasmid system (Invitrogen) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT phagemid (Stratagene), pSPORT1 plasmid (Invitrogen), or pINCY plasmid (Incyte Genomics). Recombinant plasmids were transformed into XL1-BLUE, XL1-BLUEMRF, or SOLR competent E. coli cells (Stratagene) or DH5α, DH1OB, or ELECTROMAX DH10B competent E. coli cells (Invitrogen).

In some cases, libraries were superinfected with a 5× excess of the helper phage, M13K07, according to the method of Vieira et al. (1987, Methods Enzymol 153:3-11) and normalized or subtracted using a methodology adapted from Soares (1994, Proc Natl Acad Sci 91:9228-9232), Swaroop et al. (1991, Nucleic Acids Res 19:1954), and Bonaldo et al. (1996, Genome Research 6:791-806). The modified Soares normalization procedure was utilized to reduce the repetitive cloning of highly expressed, high abundance cDNAs while maintaining the overall sequence complexity of the library. Modification included significantly longer hybridization times which allowed for increased gene discovery rates by biasing the normalized libraries toward those infrequently expressed, low-abundance cDNAs which are poorly represented in a standard transcript image (Soares, supra).

### II Isolation and Sequencing of cDNA Clones

Plasmids were recovered from host cells by in vivo excision using the UNIZAP vector system (Stratagene) or by cell lysis. Plasmids were purified using one of the following:the Magic or WIZARD MINIPREPS DNA purification system (Promega); the AGTC MINIPREP purification kit (Edge BioSystems, Gaithersburg MD); the QIAVELL 8, QIAWELL 8 Plus, or QIAWELL 8 Ultra plasmid purification systems, or the REAL PREP 96 plasmid purification kit (Qiagen). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the Calif.TALYST 800 thermal cycler (ABI) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by APB or supplied in sequencing kits such as the PRISM BIGDYE cycle sequencing kit (ABI). Electrophoretic separation of cDNA sequencing reactions and detection of labeled cDNAs were carried out using the MEGABACE 1000 DNA sequencing system (APB); the PRISM 373 or 377 sequencing systems (ABI) in conjunction with standard protocols and base calling software; or other sequence analysis systems known in the art. Reading frames within the cDNA sequences were identified using standard methods and software (reviewed in Ausubel, supra, Unit 7.7).

### III Extension of cDNA Sequences

Nucleic acid sequences were extended using the cDNA clones and oligonucleotide primers. One primer was synthesized to initiate 5′ extension of the known fragment, and the other, to initiate 3′ extension of the known fragment. The initial primers were designed using OLIGO primer analysis software (Molecular Biology Insights, Cascade Colo.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+(Stratagene) were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN reagent (0.25% reagent in 1×TE, v/v; Molecular Probes) and 0.5 μl of undiluted PCR product into each well of an opaque fluorimeter plate (Corning Costar, Acton Mass.) and allowing the DNA to bind to the reagent. The plate was scanned in a FLUOROSKAN II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC 18 vector (APB), treated with Pfu DNA polymerase (Stratagene) to fill in restriction site overhangs, and transformed into competent E. coli cells. Transformed cells were selected on antibiotic-containing media, and individual colonies were picked and cultured overnight at 37° C.in 384-well plates in LB/2× carbenicillin liquid media.

The cells were lysed and DNA was amplified by PCR using Taq DNA polymerase (APB) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

### IV Assembly and Analysis of Sequences

The nucleic acid sequences of the cDNAs presented in the Sequence Listing may contain occasional sequencing errors and unidentified nucleotides (N) that reflect state-of-the-art technology at the time the cDNA was sequenced. Occasional sequencing errors and Ns may be resolved and SNPs verified either by resequencing the cDNA or using algorithms to compare multiple sequences; these techniques are well known to those skilled in the art who wish to practice the invention. The sequences may be analyzed using a variety of algorithms described in Ausubel (supra, unit 7.7) and in Meyers (1995; Molecular Biology and Biotechnology, Wiley VCH, New York N.Y., pp. 856-853).

Component nucleotide sequences from chromatograms were subjected to PHRED analysis (Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417-424).

Processed sequences were subjected to assembly procedures in which the sequences were assigned to bins, one sequence per bin. Sequences in each bin were assembled to produce consensus sequences, referred to as “templates”. Subsequent new sequences were added to existing bins using BLAST (Altschul (supra); Altschul (1993, supra); Karlin et al. (1988) Proc Natl Acad Sci 85:841-845), BLASTn (vers.1.4, WashU), and CROSSMATCH software (Green, supra). Candidate pairs were identified as all BLAST hits having a quality score greater than or equal to 150. Alignments of at least 82% local identity were accepted into the bin. The component sequences from each bin were assembled using PHRAP (Green, supra). Bins with several overlapping component sequences were assembled using DEEP PHRAP (Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Genomics).

The assembled templates were annotated using the following procedure. Template sequences were analyzed using BLASTn (vers. 2.0, NCBI) versus GBpri (GenBank vers. 116). “Hits” were defined as an exact match having from 95% local identity over 200 base pairs through 100% local identity over 100 base pairs, or a homolog match having an E-value equal to or greater than 1×10⁻⁸ . (The “E-value” quantifies the statistical probability that a match between two sequences occurred by chance). The hits were subjected to frameshift FAST× versus GENPEPT (GenBank version 109). In this analysis, a homolog match was defined as having an E-value of 1×10⁻⁸. The assembly method used above was described in U.S. Ser. No. 09/276,534, filed Mar. 25, 1999, and the LIFESEQ GOLD user manual (Incyte Genomics).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in U.S. Ser. No. 08/812,290, filed Mar 6, 1997; U.S. Ser. No. 08/947,845, filed Oct. 9, 1997; U.S. Pat. No. 5,953,727; and U.S. Ser. No. 09/034,807, filed Mar. 4, 1998. Template sequences may be further queried against public databases such as the GenBank rodent, mammalian, vertebrate, eukaryote, prokaryote, and human EST databases.

### V Selection of Sequences, Microarray Preparation and Use

In most cases, Incyte cDNAs represent template sequences derived from the LIFESEQ GOLD assembled human sequence database (Incyte Genomics). Where more than one clone was available for a particular template, the 5′-most clone in the template was used on the microarray. The HUMAN GENOME GEM series 1-5 microarrays (Incyte Genomics) contain 45,320 array elements which represent 22,632 annotated clusters and 22,688 unannotated clusters. Table 2 shows the GenBank annotations (where available) for SEQ ID NOs:1-519 of this invention as produced by BLAST analysis.

To construct microarrays, cDNAs were amplified from bacterial cells using primers complementary to vector sequences flanking the cDNA insert. Thirty cycles of PCR increased the initial quantity of cDNAs from 1-2 ng to a final quantity greater than 5 μg. Amplified cDNAs were then purified using SEPHACRYL-400 columns (APB). Purified cDNAs were immobilized on polymer-coated glass slides. Glass microscope slides (Corning, Corning NY) were cleaned by ultrasound in 0.1% SDS and acetone, with extensive distilled water washes between and after treatments. Glass slides were etched in 4% hydrofluoric acid (VWR Scientific Products, West Chester Pa.), washed thoroughly in distilled water, and coated with 0.05% aminopropyl silane (Sigrna-Aldrich) in 95% ethanol. Coated slides were cured in a 110° C.oven. cDNAs were applied to the coated glass substrate using a procedure described in U.S. Pat. No. 5,807,522. One microliter of the cDNA at an average concentration of 100 ng/μl was loaded into the open capillary printing element by a high-speed robotic apparatus which then deposited about 5 nl of cDNA per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

### VI Preparation of Samples

Dn5797

Squamous cell carcinoma and grossly uninvolved tissue were removed from a 73 year old male donor (Roy Castle International Centre for Lung Cancer Research (RCIC), Liverpool, UK). The tumor sample showed 80% overt tumor cells within the tumor and no overtly abnormal cells in uninvolved tissue.

Dn5800

Squamous cell carcinoma and grossly uninvolved tissue were removed from a 75 year old female donor (RCIC). The tumor sample showed 70% overt tumor cells within the tumor and no overtly abnormal cells in uninvolved tissue.

Dn5793

Squamous cell carcinoma and grossly uninvolved tissue were removed from a 73 year old male donor (RCIC). The tumor sample showed 60% overt tumor cells within the tumor and no overtly abnormal cells in uninvolved tissue.

Dn5796

Squamous cell carcinoma and grossly uninvolved tissue were removed from a 66 year old male donor (RCIC). The tumor sample showed 50% overt tumor cells within the tumor and <10% overtly abnormal cells in uninvolved tissue.

Dn5792

Squamous cell carcinoma and grossly uninvolved tissue were removed from a 68 year old female donor (RCIC). The tumor sample showed 50% overt tumor cells within the tumor and no overtly abnormal cells in uninvolved tissue.

Dn5799

Adenocarcinoma and grossly uninvolved tissue were removed from a 66 year old female donor (RCIC). The tumor sample showed 80% overt tumor cells within the tumor and <5% overtly abnormal cells in uninvolved tissue.

Dn5792

Adenocarcinoma and grossly uninvolved tissue were removed from a 71 year old female donor (RCIC). The tumor sample showed 60% overt tumor cells within the tumor and <5% overtly abnormal cells in uninvolved tissue.

Each of the lung tissue samples was lysed in 1 ml of TRIZOL reagent (Invitrogen). The lysate was vortexed thoroughly, incubated at room temperature for 2-3 minutes, and extracted with 0.5 ml chloroform. The extract was mixed, incubated at room temperature for 5 minutes, and centrifuged at 15,000 rpm for 15 minutes at 4° C. The aqueous layer was collected, and an equal volume of isopropanol was added. Samples were mixed, incubated at room temperature for 10 minutes, and centrifuged at 15,000 rpm for 20 minutes at 4° C. The supernatant was removed, and the RNA pellet was washed with 1 ml of 70% ethanol, centrifuged at 15,000 rpm at 4° C., and resuspended in RNAse-free water. The concentration of the total RNA was determined by measuring the optical density at 260 nm.

Poly(A) RNA was prepared using an OLIGOTFX mRNA kit (Qiagen) with the following modifications: OLIGOTEX beads were washed in tubes instead of on spin columns, resuspended in elution buffer, and then loaded onto spin columns to recover MRNA. To obtain maximum yield, the mRNA was eluted twice.

Each poly(A) RNA sample was reverse transcribed using a cDNA synthesis system (Invitrogen) with Not I-T7-VN primers (5′ GCATTAGCGGCCGCGAAATTAATACGACTCACTATAGGGAGAT TTTTTTTTTTTTTTTTVN 3′) and 100 units MMLV RNAseH (−) reverse-transcriptase (Progmega) in the first strand reaction. The resulting cDNA was purified on a CHROMASPIN TE-200 column (Clontech) and lyophilized until dry. The cDNA was amplified 200-400 fold using an AMPLISCRIBE IVT kit (Epicentre Technologies, Madison Wis.) in a procedure modified from U.S. Pat. No. 5,716,785 and U.S. Pat. No. 5,891,636. The amplified RNA was purified on a CHROMASPIN DEPC-200 column (Clontech).

Amplified RNA was labeled using MMLV reverse-transcriptase, random primer (9mer), 1× first strand buffer, 0.03 units/μl RNAse inhibitor, 500 μM dATP, 500 μM dGTP, 500 μM dTTP, 40 μM dCTP, and 40 μM either dCTP-Cy3 or dCTP-Cy5 (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mnRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

cDNAs were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml sodium acetate, and 300 ml of 100% ethanol. The cDNAs were then dried to completion using a SPEEDVAC system (Savant Instruments, Holbrook N.Y.) and resuspended in 14 ,μl 5× SSC/0.2% SDS.

### VII Hybridization and Detection

Competitive hybridization reactions compared cDNAs derived from tumor tissue and uninvolved tissue from the same donor. cDNA from all donors except Dn5795 were hybridized to HUMAN GENOME GEMs 1-5. cDNAs from Dn5795 were hybridized to HUMAN GENOME GEM 1.

Hybridization reactions contained 9 μl of sample mixture containing 0.2 μg each of Cy3 and Cy5 labeled cDNA synthesis products in 5× SSC, 0.2% SDS hybridization buffer. The mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5× SSC in a corner of the chamber. The chamber containing the microarrays was incubated for about 6.5 hours at 60° C. The microarrays were washed for 10 min at 45° C. in low stringency wash buffer (1× SSC, 0.1% SDS), three times for 10 minutes each at 45° C. in high stringency wash buffer (0.1× SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light was focused on the microarray using a 20× microscope objective (Nikon, Melville N.Y.). The slide containing the microarray was placed on a computer-controlled X-Y stage on the microscope and raster-scanned past the objective. The 1.8 cm ×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Genomics). Significance was defined as signal to background ratio exceeding 2× and area hybridization exceeding 40%.

### VIII Data Analysis and Results

Array elements that exhibited a 2-fold or greater change in expression at one or more time points, a signal intensity over 250 units, a signal-to-background ratio of at least 2.5, and an element spot size of at least 40% were identified as differentially expressed using the GEMTOOLS program (Incyte Genomics). Differential expression values are in log base 2 scale. The cDNAs that were differentially expressed are shown in Table 1 and are identified by SEQ ID NO, Template ID, and Clone ID. Negative values represent upregulation in tumor tissue.

### IX Further Characterization of Differentially Expressed cDNAs and Proteins

Clones were aligned against the LIFESEQ GOLD 5.1 database (January 2001 EST data release; Incyte Genomics) using BLAST analysis and an Incyte template was chosen for each clone. The template was aligned against GenBank database using BLAST analysis to acquire annotation. The nucleotide sequences were translated into amino acid sequences which were aligned against GenPept and other protein databases to acquire annotation and characterization, i.e., structural motifs. The cDNAs that were differentially expressed are shown in Table 2 and are identified by SEQ ID NO, Template ID, Clone ID, and by the description associated with at least a fragment of a polynucleotide found in GenBank. The descriptions were obtained using the sequences of the Sequence Listing and BLAST analysis. Different templates identified in Table 2 may share an identical GenBank annotation. These templates represent related homologs or splice variants. Templates with no similarity to a sequence in the GenBank database are identified in Table 2 as “Incyte Unique”.

Percent sequence identity can be determined electronically for two or more amino acid or nucleic acid sequences using the MEGALIGN program, a component of LASERGENE software (DNASTAR). The percent identity between two amino acid sequences is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage identity.

Sequences with conserved protein motifs may be searched using the BLOCKS search program. This program analyses sequence information contained in the SWISSPROT and PROSITE databases and is useful for determining the classification of uncharacterized proteins translated from genomic or cDNA sequences (Bairoch, supra; Attwood, supra). PROSITE database is a useful source for identifying functional or structural domains that are not detected using motifs due to extreme sequence divergence. Using weight matrices, these domains are calibrated against the SWISSPROT database to obtain a measure of the chance distribution of the matches.

The PRINTS database can be searched using the BLIMPS search program to obtain protein family “fingerprints”. The PRINTS database complements the PROSITE database by exploiting groups of conserved motifs within sequence alignments to build characteristic signatures of different protein families. For both BLOCKS and PRINTS analyses, the cutoff scores for local similarity were: >1300=strong, 1000-1300=suggestive; for global similarity were: p<exp-3; and for strength (degree of correlation) were: >1300=strong, 1000-1300=weak. Pfam is a large collection of multiple sequence alignments and hidden Markov models covering many common protein domains. Version 5.5 of Pfam (September 2000) contains alignments and models for 2478 protein families, based on the SWISSPROT 38 and SP-TrEMBL 11 protein sequence databases.

### X Other Hybridization Technologies and Analyses

Other hybridization technologies utilize a variety of substrates such as nylon membranes, capillary tubes, etc. Arranging cDNAs on polymer coated slides is described in Example V; sample cDNA preparation, hybridization, and analysis using polymer coated slides is described in examples VI and VII, respectively.

The cDNAs are applied to a membrane substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hours. The membrane is removed from the agar and consecutively placed colony side up in 10% SDS, denaturing solution (1.5 M NaCl, 0.5 M NaOH ), neutralizing solution (1.5 M NaCl, 1 M Tris, pH 8.0), and twice in 2× SSC for 10 minutes each. The membrane is then UV irradiated in a STRATALINKER UV-crosslinker (Stratagene). In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPRACRYL-400 beads (APB). Purified nucleic acids are arranged on a nylon membrane manually or using a dot/slot blotting manifold and suction device and are immobilized by denaturation, neutralization, and WV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl Tris-EDTA (ethylenediamine tetraacetic acid) (TE) buffer, denaturing by heating to 100° C. for five minutes and centrifuging briefly. The denatured cDNA is then added to a REDIPRIME tube (APB), gently mixed until blue color is evenly distributed, and centrifuged briefly. Five microliters of [³²P]dCTP is added to the tube, and the contents are incubated at 37° C. for 10 minutes. The labeling reaction is stopped by adding 5 ,μl of 0.2M EDTA, and probe is purified from unincorporated nucleotides using a PROBEQUANT G-50 microcolumn (APB). The purified probe is heated to 100° C. for five minutes and then snap cooled for 2 minutes on ice.

Membranes are pre-hybridized in hybridization solution containing 1% Sarkosyl and 1× high phosphate buffer (0.5 M NaCl, 0.1 M Na₂HPO₄, 5 mM EDTA, pH 7) at 55° C. for 2 hours. The diluted in 15 ml fresh hybridization solution, is then added to the membrane. The membrane is hybridized with the probe at 55° C. for 16 hours. Following hybridization, the membrane is washed for 15 minutes at 25° C. in 1 mM Tris (pH 8.0), 1% Sarkosyl, and four times for 15 minutes each at 25° C. in 1 mM Tris (pH 8.0). To detect hybridization complexes, XOMAT-AR film (Eastman Kodak, Rochester N.Y.) is exposed to the membrane overnight at −70° C., developed, and examined.

### XI Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with IPTG. Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB). The fusion protein is purified on immobilized glutathione under conditions that maintain protein activity and antigenicity. After purification, the GST moiety is proteolytically cleaved from the protein with thrombin. Alternatively, a fusion protein antigenically tagged with FLAG, an 8-amino acid peptide, is purified using commercially available monoclonal and polyclonal anti-FLAG antibodies (Eastman Kodak, Rochester N.Y.).

### XII Production of Specific Antibodies

A denatured protein from a reverse phase HPLC separation is obtained in quantities up to 75 mg. This denatured protein is used to immunize mice or rabbits following standard protocols. About 100 ,μg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to select regions of high antigenicity, antigenically-effective epitopes of the protein. The optimal sequences for immunization are usually at the C-terminus, the N-terminus, and those intervening hydrophilic regions of the protein that are likely to be exposed to the external environment when the protein is in its natural conformation. Typically, oligopeptides about 15 residues in length are synthesized using a 431 peptide synthesizer (ABI) using Fmoc-chemistry and then coupled to keyhole limpet hemocyanin (KLH; Sigma-Aldrich) by reaction with M-maleimidobenzoyl-N-hydroxysuccinimide ester. If necessary, a cysteine may be introduced at the N-termninus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA, washed, and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

### XIII Purification of Naturally Occurring Protein Using Specific Antibodies

Naturally occurring or recombinant protein is immunopurified by affinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

### XIV Screening Molecules for Specific Binding with the cDNA or Protein

The cDNA or fragments thereof and the protein or portions thereof are labeled with ³²P-dCTP, Cy3-dCTP, Cy5-dCTP (APB), or BIODIPY or FITC (Molecular Probes), respectively. Candidate molecules or compounds previously arranged on a substrate are incubated in the presence of labeled nucleic or amino acid. After incubation under conditions for either a cDNA or a protein, the substrate is washed, and any position on the substrate retaining label, which indicates specific binding or complex formation, is assayed. The binding molecule is identified by its arrayed position on the substrate. Data obtained using different concentrations of the nucleic acid or protein are used to calculate affinity between the labeled nucleic acid or protein and the bound molecule. High throughput screening using very small assay volumes and very small amounts of test compound is fully described in U.S. Pat. No. 5,876,946.

All patents and publications mentioned in the specification are incorporated herein by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ ID | | | Avg | Avg | Avg | Avg | Avg | Avg | Avg | |
|---|---|---|---|---|---|---|---|---|---|---|
| NO | Template ID | Clone ID | Dn5797 | Dn5800 | Dn5793 | Dn5796 | Dn5792 | Dn5799 | Dn5795 | TTEST |
| 1 | 1867417CB1 | 1357231 | −2.81 | −2.71 | −2.25 | −3.08 | −3.66 | −1.90 | | |
| 2 | 1970111CB1 | 1970111 | −1.79 | −2.10 | −1.80 | −2.00 | −3.97 | −1.86 | | |
| 3 | 959142CB1 | 1930447 | −2.74 | −1.72 | −1.98 | −2.73 | −1.81 | −3.14 | −1.61 | 0.855 |
| 4 | 064684.7 | 1824717 | −3.64 | −1.59 | −2.95 | −3.67 | −3.14 | −3.63 | | |
| 5 | 245093.34 | 958486 | −3.91 | −3.82 | −3.69 | −3.34 | −1.78 | −3.18 | | |
| 6 | 331908.5 | 1995457 | −3.44 | −2.23 | −1.67 | −1.91 | −0.20 | −2.22 | −1.65 | 0.9425 |
| 7 | 1329880.35 | 5150602 | −4.05 | −1.87 | −4.15 | −4.43 | −2.05 | −4.14 | −2.00 | 0.8648 |
| 8 | 963555.1 | 2820294 | −4.14 | −2.15 | −4.96 | −4.86 | −1.74 | −4.51 | | |
| 9 | 199471.2 | 2047549 | −2.60 | −2.48 | −1.51 | −3.80 | −4.01 | −1.69 | | |
| 10 | 048849.1 | 1358605 | −3.21 | −1.56 | −2.34 | −3.34 | −1.35 | −3.20 | −1.51 | 0.9964 |
| 11 | 278283.1 | 4180161 | −4.06 | −1.94 | −4.23 | −4.63 | −1.87 | −4.29 | −2.15 | 0.929 |
| 12 | 978433CB1 | 1869068 | −4.49 | −1.65 | −3.35 | −3.48 | −3.38 | −4.46 | −2.44 | 0.8909 |
| 13 | 611514CB1 | 605219 | 2.62 | 2.10 | 2.12 | 2.52 | 2.92 | 2.52 | | |
| 14 | 1382907.35 | 1888708 | 1.79 | 1.98 | 2.12 | 0.42 | 2.39 | 2.15 | 1.65 | 0.7242 |
| 15 | 350509.2 | 1510413 | 1.91 | 2.25 | 1.85 | 3.18 | 3.00 | 2.00 | 1.23 | 0.2156 |
| 16 | 2512879CB1 | 2512879 | 3.06 | 2.19 | 1.61 | 2.41 | 2.71 | 2.94 | | |
| 17 | 241123.1 | 3876732 | 1.42 | 1.83 | 2.15 | 1.76 | 2.71 | 1.59 | 1.85 | 0.3632 |
| 18 | 247817.4 | 1480063 | 1.85 | 1.29 | 2.36 | 2.43 | 2.29 | 1.51 | 1.61 | 0.0862 |
| 19 | 1674368CB1 | 1241484 | 2.17 | 1.43 | 0.96 | 2.75 | 2.22 | 2.09 | 2.07 | 0.6117 |
| 20 | 343963.1 | 2671006 | 2.68 | 1.86 | 1.63 | 2.08 | 2.49 | 1.20 | 1.96 | 0.3444 |
| 21 | 273154CB1 | 2899786 | 2.63 | 4.16 | 1.65 | 4.44 | 3.51 | 2.09 | 0.85 | 0.1263 |
| 22 | 331508.4 | 1967759 | 1.98 | 0.58 | 1.80 | 1.94 | 1.80 | 1.61 | 2.25 | 0.5177 |
| 23 | 1137924.1 | 1480479 | 2.41 | 1.56 | 2.00 | 2.21 | 1.41 | 0.90 | 1.68 | 0.3208 |
| 24 | 247168.4 | 2278925 | 2.26 | 1.43 | 1.89 | 1.81 | 2.56 | 2.15 | 1.00 | 0.5995 |
| 25 | 2275817CB1 | 224996 | 1.96 | 0.83 | 1.58 | 1.68 | 1.75 | 1.63 | 1.40 | 0.8492 |
| 26 | 2717806CB1 | 4918603 | 2.52 | 1.63 | 2.02 | 1.65 | 1.80 | 2.17 | | |
| 27 | 407624.1 | 3771805 | 1.83 | 1.43 | 1.68 | 1.43 | 1.41 | 2.00 | | |
| 28 | 3950154CB1 | 2748163 | 2.12 | 1.10 | 1.38 | 2.51 | 2.61 | 1.96 | 2.35 | 0.5842 |
| 29 | 3741842CB1 | 1988080 | 1.65 | 0.43 | 2.23 | 2.51 | 2.03 | 1.46 | 2.14 | 0.9552 |
| 30 | 221055.4 | 2718391 | 2.69 | 1.20 | 2.25 | 1.68 | 2.10 | 2.40 | 1.56 | 0.9943 |
| 31 | 379425CB1 | 433622 | 1.70 | 1.43 | 1.96 | 2.23 | 3.19 | 1.58 | 0.26 | 0.2891 |
| 32 | 1685090CB1 | 2721792 | 3.48 | 2.66 | 3.75 | 1.74 | 3.72 | 3.22 | 3.42 | 0.5619 |
| 33 | 350476.1 | 123312 | 3.47 | −0.07 | 3.55 | 3.02 | 2.68 | 2.84 | 3.30 | 0.4814 |
| 33 | 350476.1 | 4044520 | 3.39 | −0.26 | 4.50 | 3.89 | 3.69 | 2.76 | | |
| 34 | 404278.1 | 3116479 | 2.22 | 2.09 | 1.63 | 2.19 | 2.84 | 1.77 | | |
| 35 | 1250492CB1 | 1361644 | 2.34 | 2.78 | 2.10 | 2.79 | 3.64 | 2.26 | | |
| 36 | 200095.2 | 3125685 | 2.02 | 2.12 | 1.35 | 2.08 | 2.49 | 1.80 | −0.63 | 0.4459 |
| 37 | 997405.3 | 1240444 | 4.92 | 3.90 | 3.14 | 4.13 | 4.42 | 4.65 | 4.25 | 0.3769 |
| 38 | 235369.10 | 1965041 | 2.55 | 2.07 | 2.29 | 2.98 | 2.70 | 1.91 | 0.43 | 0.3093 |
| 39 | 236587.4 | 1362125 | 3.07 | 2.22 | 2.26 | 3.57 | 2.87 | 2.60 | 0.00 | 0.4502 |
| 40 | 1135936.1 | 4271973 | 2.67 | 2.28 | 3.37 | 3.54 | 3.96 | 3.13 | 4.26 | 0.5115 |
| 41 | 251123.6 | 3557441 | 1.65 | 1.70 | 1.77 | 1.57 | 1.53 | 1.77 | 1.58 | 0.8036 |
| 41 | 251123.6 | 2600963 | 1.56 | 2.34 | 2.06 | 1.87 | 1.12 | 2.04 | | |
| 42 | 2547002CB1 | 2475740 | 2.49 | 1.14 | 2.61 | 1.43 | 1.75 | 1.54 | 2.99 | 0.6935 |
| 43 | 251123.8 | 3557441 | 1.65 | 1.70 | 1.77 | 1.57 | 1.53 | 1.77 | 1.58 | 0.8036 |
| 44 | 1000172.35 | 3645309 | 3.61 | 2.42 | 2.52 | 3.58 | 3.16 | 3.38 | 2.72 | 0.9862 |
| 45 | 2921920CB1 | 1498363 | 3.29 | 0.48 | 2.77 | 2.09 | 2.60 | 1.88 | 3.99 | 0.6319 |
| 46 | 631645CB1 | 1274935 | 2.36 | 1.68 | 1.17 | 2.48 | 1.64 | 2.06 | 1.48 | 0.8188 |
| 46 | 631645CB1 | 1577756 | 1.56 | 1.13 | 0.72 | 1.89 | 1.38 | 1.51 | | |
| 47 | 1507546CB1 | 3507734 | 2.50 | 2.50 | 1.80 | 2.22 | 3.10 | 2.75 | | |
| 48 | 1508437CB1 | 1376121 | 2.53 | 2.60 | 1.88 | 3.00 | 2.96 | 1.74 | 1.17 | 0.0764 |
| 48 | 1508437CB1 | 1607091 | 2.97 | 2.92 | 2.63 | 2.96 | 3.44 | 2.25 | | |
| 49 | 1100669.4 | 1675122 | 2.20 | 1.29 | 1.67 | 2.90 | 2.33 | 1.74 | 2.05 | 0.5901 |
| 50 | 124600CB1 | 1578941 | 3.12 | 0.38 | 3.23 | 2.03 | 3.22 | 2.38 | 2.94 | 0.6875 |
| 51 | 470771.11 | 322066 | 1.51 | 1.52 | 1.74 | 1.68 | 1.94 | 1.84 | 1.51 | 0.9888 |
| 52 | 273259.4 | 2201411 | 2.26 | 1.41 | 1.91 | 1.98 | 1.38 | 0.60 | 1.58 | 0.3717 |
| 53 | 5170638CB1 | 4014022 | 2.52 | 1.51 | 2.64 | 2.62 | 2.15 | 2.06 | 2.71 | 0.8267 |
| 54 | 1330170.3 | 4221057 | 2.15 | 2.50 | 2.46 | 3.22 | 3.81 | 2.32 | | |
| 55 | 1383898.2 | 1499549 | 2.22 | 1.17 | 1.65 | 1.65 | 1.94 | 1.71 | 1.58 | 0.6827 |
| 56 | 019238.3 | 1430507 | 2.79 | 1.53 | 2.00 | 2.46 | 2.56 | 2.18 | 1.29 | 0.4234 |
| 57 | 1102296.5 | 3026658 | 2.20 | 2.88 | 3.58 | 3.84 | 4.97 | 2.39 | | |
| 58 | 995673.3 | 2149968 | 2.15 | 3.10 | 3.21 | 3.48 | 4.56 | 2.16 | | |
| 58 | 995673.3 | 4215545 | 2.26 | 2.53 | 2.91 | 3.34 | 4.26 | 2.17 | | |
| 59 | 312256CB1 | 313697 | 1.71 | 2.02 | 1.91 | 1.81 | 2.23 | 1.94 | 1.72 | 0.5193 |
| 60 | 199183.2 | 2763310 | 2.34 | 2.74 | 2.67 | 2.63 | 2.99 | 2.13 | −1.29 | 0.4125 |
| 61 | 289671.40 | 3678546 | 3.19 | 2.29 | 3.02 | 3.89 | 3.10 | 2.42 | | |
| 62 | 154403.1 | 2050104 | 2.12 | 2.68 | 2.17 | 2.93 | 4.02 | 1.98 | 2.92 | 0.6197 |
| 63 | 086518.22 | 1987759 | 2.47 | 1.51 | 2.20 | 2.42 | 2.51 | 1.94 | | |
| 64 | 1383156.10 | 311197 | 4.70 | 5.83 | 3.18 | 6.64 | 5.29 | 5.17 | 5.83 | 0.6026 |
| 65 | 1100669.3 | 1675122 | 2.20 | 1.29 | 1.67 | 2.90 | 2.33 | 1.74 | 2.05 | 0.5901 |
| 66 | 1330148.1 | 3867795 | 1.89 | 2.73 | 2.26 | 2.23 | 3.38 | 1.54 | 2.59 | 0.5565 |
| 67 | 1436702CB1 | 1358185 | 1.98 | 1.87 | 1.91 | 2.06 | 2.05 | 1.48 | 2.10 | 0.6587 |
| 68 | 3141226CB1 | 3625857 | 2.42 | −0.19 | 2.17 | 4.13 | 4.30 | 1.91 | 1.89 | 0.4588 |
| 69 | 289671.44 | 970905 | 2.34 | 2.02 | 2.34 | 3.60 | 2.58 | 1.83 | 2.02 | 0.0755 |
| 70 | 197927.7 | 2417149 | 2.68 | 1.72 | 2.38 | 2.66 | 3.10 | 2.28 | | |
| 71 | 1134834.2 | 1911306 | 2.08 | 1.46 | 1.96 | 2.08 | 1.19 | 1.86 | 2.25 | 0.3424 |
| 72 | 200273.1 | 2721144 | 2.77 | 2.57 | 2.79 | 3.29 | 2.42 | 2.15 | | |
| 73 | 477974.1 | 3117677 | 2.10 | 2.34 | 1.77 | 2.41 | 3.02 | 2.39 | | |
| 74 | 235095.7 | 1809377 | 2.10 | 1.77 | 1.91 | 1.32 | 2.22 | 1.96 | 1.56 | 0.7161 |
| 75 | 407593.1 | 205053 | 1.41 | 1.92 | 2.02 | 2.26 | 3.41 | 1.46 | | |
| 76 | 332301.1 | 1417150 | 2.12 | 1.51 | 1.91 | 1.72 | 1.81 | 2.29 | | |
| 77 | 407699.4 | 1919860 | 2.05 | 1.61 | 2.25 | 1.83 | 2.41 | 2.05 | | |
| 78 | 405501.1 | 2593077 | 2.63 | 2.66 | 2.34 | 3.25 | 3.20 | 1.56 | | |
| 79 | 482541.2 | 2685454 | 2.35 | 1.84 | 1.90 | 2.56 | 3.00 | 1.96 | | |
| 80 | 237026.3 | 2963374 | 2.02 | 2.25 | 1.96 | 2.55 | 2.55 | 2.23 | | |
| 81 | 443605.4 | 3556587 | 3.26 | 3.38 | 2.49 | 2.92 | 3.32 | 2.87 | 0.32 | 0.4506 |
| 82 | 443605.15 | 3556587 | 3.26 | 3.38 | 2.49 | 2.92 | 3.32 | 2.87 | 0.32 | 0.4506 |
| 83 | 029997.1 | 3638532 | 1.87 | 2.26 | 1.65 | 2.09 | 3.10 | 3.21 | 1.77 | 0.7534 |
| 84 | 331743.9 | 789903 | −1.70 | −1.39 | −1.20 | −1.85 | −2.25 | −1.59 | | |
| 85 | 337334.1 | 3220029 | −1.23 | −1.48 | −0.48 | −1.56 | −1.37 | −0.81 | 0.20 | 0.292 |
| 86 | 410438.2 | 1879818 | 1.96 | 1.72 | 1.26 | 0.60 | 1.83 | 0.13 | 1.54 | 0.5245 |
| 87 | 453004.32 | 2394888 | 1.58 | 1.46 | 1.07 | 1.96 | 1.85 | 1.35 | 0.38 | 0.3609 |
| 88 | 236319.2 | 3044230 | 1.23 | 0.38 | 1.06 | 1.08 | 1.56 | 0.81 | | |
| 89 | 978118.5 | 2508618 | 2.62 | 0.43 | 1.96 | 3.07 | 1.62 | 1.92 | | |
| 90 | 1383177.16 | 1646505 | 1.53 | 0.00 | 1.35 | 1.48 | 2.15 | 1.26 | 1.51 | 0.834 |
| 91 | 1383298.1 | 1510539 | 1.86 | 1.04 | 0.63 | 0.77 | 2.45 | 1.22 | 2.22 | 0.6032 |
| 92 | 1000222.20 | 1729693 | −1.14 | −1.29 | −0.89 | −1.10 | −1.99 | −0.85 | | |
| 93 | 1096863.27 | 1303863 | 1.59 | 0.26 | 1.17 | 1.77 | 0.68 | 1.10 | | |
| 94 | 245334.1 | 2380412 | −3.74 | −2.63 | −1.30 | −3.15 | 0.00 | −2.79 | 0.07 | 0.6767 |
| 95 | 1656674CB1 | 2222802 | 1.40 | 1.67 | 0.76 | 2.08 | 2.33 | 1.34 | 1.32 | 0.3106 |
| 96 | 522678CB1 | 2199851 | −1.48 | −1.04 | −1.83 | −0.14 | −0.99 | −1.42 | | |
| 97 | 1424985CB1 | 2150615 | 1.26 | −0.26 | 1.32 | 0.68 | 1.66 | 1.29 | | |
| 98 | 1468237CB1 | 1573505 | −1.29 | −1.40 | −0.63 | −1.63 | −1.63 | −0.72 | −0.43 | 0.0325 |
| 99 | 337008.1 | 1599272 | 1.85 | 1.23 | 1.32 | −0.88 | 1.58 | 1.65 | 1.70 | 0.2501 |
| 100 | 444850.9 | 2860704 | 1.68 | 1.10 | 1.14 | 1.14 | 0.72 | −0.63 | | |
| 101 | 786284CB1 | 1458210 | 1.87 | 0.85 | 1.96 | 2.37 | 2.06 | 1.07 | 0.58 | 0.0586 |
| 102 | 1352170CB1 | 1988092 | 1.79 | 1.17 | 1.61 | 0.63 | 2.68 | 0.96 | | |
| 103 | 1815320CB1 | 1742116 | 1.14 | −0.38 | 1.61 | 2.54 | 1.56 | 1.10 | 0.32 | 0.3969 |
| 104 | 331192.11 | 2007554 | −0.31 | −1.63 | −2.10 | −3.08 | −3.60 | −1.28 | | |
| 105 | 344053.5 | 2928545 | −2.07 | 1.10 | −1.93 | −1.38 | 0.07 | −2.47 | −1.46 | 0.2316 |
| 106 | 411188.2 | 4247796 | −2.73 | 2.05 | −4.03 | −1.49 | 0.20 | −3.56 | | |
| 107 | 331510.4 | 3814138 | −2.07 | 1.72 | −3.15 | −1.17 | 0.72 | −2.36 | | |
| 108 | 492750CB1 | 2732630 | −1.07 | −1.29 | −0.85 | −1.65 | −1.74 | −0.67 | | |
| 109 | 348912.4 | 1716655 | 2.70 | 2.31 | 1.80 | 1.65 | 2.87 | 0.43 | −1.92 | 0.2253 |
| 110 | 2680109CB1 | 2242677 | 1.59 | 1.61 | 1.23 | 1.72 | 1.98 | 1.54 | −0.81 | 0.4763 |
| 110 | 2680109CB1 | 2125020 | 1.20 | 1.03 | 0.72 | 0.93 | 1.60 | 1.29 | | |
| 111 | 512261CB1 | 512261 | 1.29 | 0.67 | 1.37 | 1.93 | 1.53 | 0.93 | 1.43 | 0.6267 |
| 112 | 232719.2 | 1848259 | 1.14 | 0.32 | 1.59 | 1.10 | 1.49 | 0.80 | 1.26 | 0.7793 |
| 113 | 369664.2 | 3119171 | −2.20 | 2.45 | −2.74 | −1.35 | 0.43 | −2.62 | −1.63 | 0.2357 |
| 114 | 021042.1 | 2879922 | −3.02 | −1.32 | −2.62 | −3.18 | 0.07 | −2.82 | −1.04 | 0.945 |
| 115 | 418805.7 | 1637320 | 1.16 | 1.07 | 1.43 | 1.14 | 2.23 | 0.53 | | |
| 116 | 1094000.4 | 1397816 | 1.94 | −0.12 | 1.98 | 0.00 | 1.80 | 2.17 | | |
| 117 | 1094000.5 | 1380927 | 2.94 | 0.48 | 3.50 | 2.51 | 2.97 | 2.86 | | |
| 118 | 1222734CB1 | 1300701 | 2.02 | 0.13 | 2.08 | 0.00 | 1.90 | 2.17 | | |
| 118 | 1222734CB1 | 1397816 | 1.94 | −0.12 | 1.98 | 0.00 | 1.80 | 2.17 | | |
| 119 | 411205.5 | 1420883 | −2.06 | −2.10 | −0.76 | −3.13 | −2.98 | −1.68 | | |
| 120 | 1092777.6 | 1643711 | 1.29 | 0.43 | 1.46 | 1.82 | 1.28 | 0.99 | 1.51 | 0.9874 |
| 121 | 234358.5 | 1996180 | −1.35 | −1.34 | −0.72 | −2.48 | −2.71 | −1.31 | 0.26 | 0.3401 |
| 122 | 1092777.7 | 1643711 | 1.29 | 0.43 | 1.46 | 1.82 | 1.28 | 0.99 | 1.51 | 0.9874 |
| 123 | 4180444CB1 | 3872317 | 1.23 | 1.23 | 0.53 | 1.29 | 1.25 | 1.17 | 1.54 | 0.3847 |
| 124 | 1987983CB1 | 690819 | 1.76 | 1.34 | 1.00 | 1.45 | 1.53 | 1.07 | 1.61 | 0.8297 |
| 125 | 1398420.2 | 4655050 | 2.19 | 1.82 | 1.85 | 1.32 | 2.18 | 0.53 | −1.04 | 0.2136 |
| 126 | 1749102CB1 | 1749102 | 3.32 | 2.23 | 2.09 | 2.44 | −1.54 | 0.48 | 1.17 | 0.3762 |
| 127 | 2965804CB1 | 2965804 | 1.28 | 0.20 | 2.26 | 1.64 | 3.31 | 1.54 | | |
| 128 | 210095.21 | 1285926 | 2.29 | 1.26 | 1.87 | 1.07 | 2.51 | 0.53 | | |
| 129 | 1555752CB1 | 42248 | −1.14 | −1.10 | 0.00 | −1.98 | −2.54 | −0.29 | | |
| 130 | 110245.1 | 3028719 | −1.04 | −1.23 | 0.07 | −1.84 | −1.60 | −0.43 | 0.34 | 0.1341 |
| 131 | 009476CB1 | 2017386 | −1.51 | −0.96 | −0.72 | −1.96 | −1.85 | −1.43 | | |
| 132 | 1560874CB1 | 3815942 | −1.46 | −1.20 | −0.67 | −1.74 | −1.53 | −0.38 | −0.13 | 0.0061 |
| 133 | 3571894CB1 | 2132715 | −1.04 | −0.58 | −0.68 | −1.28 | 1.74 | −1.72 | | |
| 134 | 349622.1 | 2055903 | −3.15 | −1.11 | −2.05 | −5.27 | −0.24 | −0.85 | | |
| 135 | 002940CB1 | 1679482 | −1.77 | −1.30 | −0.58 | −2.21 | −2.01 | −0.29 | 0.13 | 0.0114 |
| 136 | 1362466CB1 | 1834502 | −1.03 | −1.38 | −0.92 | −2.12 | −2.20 | −1.23 | 0.58 | 0.3975 |
| 137 | 383376.19 | 4461157 | 1.74 | 1.00 | 1.17 | 1.89 | 2.05 | 0.76 | 1.65 | 0.558 |
| 138 | 1382961.3 | 3184882 | −4.32 | −1.91 | −1.58 | −3.76 | 0.58 | −0.20 | 0.37 | 0.0581 |
| 139 | 1382961.15 | 4382348 | −5.07 | −4.05 | −3.81 | −5.39 | 1.14 | −0.13 | | |
| 140 | 1454852CB1 | 793403 | −3.01 | −2.51 | −1.32 | −2.42 | −0.14 | −0.26 | 0.38 | 0.0253 |
| 141 | 2161632CB1 | 954057 | −1.78 | −1.72 | −0.58 | −1.59 | −0.68 | −1.32 | 0.19 | 0.5145 |
| 142 | 2058013CB1 | 1968413 | −3.63 | −1.24 | −2.30 | −3.70 | 0.72 | −0.20 | | |
| 143 | 235333.1 | 1684632 | 2.16 | 0.81 | 2.32 | 2.03 | 2.58 | 1.35 | | |
| 144 | 702628CB1 | 2418629 | −1.35 | −1.70 | −1.46 | −2.31 | −2.05 | −1.10 | | |
| 145 | 1383415.3 | 1420380 | −1.98 | −1.14 | −0.68 | −2.87 | −2.37 | 0.00 | 0.19 | 0.0077 |
| 146 | 236309.1 | 2925376 | 1.58 | 1.26 | 0.63 | 0.71 | 1.30 | 1.29 | | |
| 147 | 344868.13 | 3333118 | −0.48 | −1.04 | −1.00 | −1.07 | −1.51 | −1.23 | | |
| 148 | 368869.1 | 5100602 | −1.93 | 2.26 | −2.83 | −1.49 | 0.13 | −2.38 | | |
| 149 | 3256566CB1 | 1666069 | −2.39 | −1.72 | −0.43 | −1.80 | 0.26 | −1.48 | | |
| 150 | 4104673CB1 | 4104673 | −3.20 | −1.56 | −0.72 | −4.70 | 1.93 | −2.75 | | |
| 151 | 345511.2 | 4243318 | 2.12 | 2.02 | 1.65 | 2.22 | 2.07 | 1.20 | | |
| 152 | 1723834CB1 | 1723834 | −2.56 | −1.59 | −1.35 | −1.44 | −3.11 | −0.77 | −0.48 | 0.0151 |
| 153 | 078756CB1 | 490795 | 3.16 | 0.00 | 2.94 | 1.22 | 2.33 | 2.08 | 2.75 | 0.5062 |
| 154 | 331749.3 | 1628341 | −4.41 | −3.57 | −0.85 | −4.02 | 0.24 | −1.48 | 0.26 | 0.2185 |
| 155 | 350528.7 | 1429293 | 1.56 | 0.53 | 0.96 | 1.44 | 1.51 | 1.35 | 1.91 | 0.3306 |
| 156 | 1099159.1 | 1519404 | −2.47 | −1.35 | −1.92 | −3.39 | −0.26 | −2.32 | −0.05 | 0.6525 |
| 157 | 1530186CB1 | 3715059 | 1.31 | 1.22 | 1.89 | 3.14 | 1.94 | 1.51 | 1.46 | 0.2931 |
| 158 | 1092387.11 | 2674527 | −3.22 | −1.49 | −0.26 | −4.28 | 0.96 | −2.93 | | |
| 159 | 107939.2 | 2313581 | 1.29 | 0.37 | 1.34 | 1.03 | 1.13 | 0.72 | 1.68 | 0.788 |
| 160 | 899248.6 | 1832584 | 1.61 | 1.03 | 2.12 | 1.10 | 1.61 | 0.14 | | |
| 161 | 2959521CB1 | 87727 | −1.38 | −1.69 | −0.43 | −1.29 | −0.26 | −1.35 | | |
| 162 | 3331519CB1 | 2197965 | 1.91 | 0.77 | 1.73 | 2.33 | 1.26 | 1.00 | 1.56 | 0.4695 |
| 163 | 1453334CB1 | 1445547 | −2.10 | −2.42 | −0.96 | −1.65 | 0.00 | −1.89 | −0.53 | 0.815 |
| 164 | 2798854CB1 | 4385292 | −1.26 | −1.60 | −0.43 | −2.55 | −1.11 | 0.00 | | |
| 165 | 227961.1 | 2742979 | −1.10 | −1.17 | −0.58 | −2.47 | −1.98 | −1.00 | −0.14 | 0.2247 |
| 166 | 1427470CB1 | 1430862 | 2.12 | 0.48 | 1.17 | 0.24 | 1.43 | 1.51 | 1.38 | 0.354 |
| 167 | 206250.6 | 2180220 | 2.17 | 1.23 | 0.76 | 1.69 | 2.32 | 1.50 | 1.63 | 0.8272 |
| 168 | 995068.15 | 893230 | 1.43 | 1.17 | 1.60 | 1.87 | 0.00 | 1.10 | 1.74 | 0.6796 |
| 169 | 2545475CB1 | 1981569 | −2.76 | −2.44 | −0.63 | −1.91 | −1.59 | −1.17 | 0.19 | 0.2439 |
| 170 | 1502559CB1 | 1502559 | 1.43 | −0.58 | 1.26 | 1.38 | 1.50 | 0.89 | | |
| 171 | 2239738CB1 | 2239738 | 1.85 | 2.38 | 1.17 | 1.17 | 1.45 | 1.10 | 0.43 | 0.1717 |
| 172 | 347572.1 | 1501621 | 1.26 | 1.35 | 1.26 | 0.54 | 1.48 | −1.06 | 0.14 | 0.2033 |
| 173 | 977985.10 | 1445387 | 1.17 | 1.14 | 2.00 | 2.31 | 1.41 | 0.93 | | |
| 174 | 236582.2 | 2720359 | 2.17 | 1.97 | 1.48 | 1.91 | 1.91 | 0.96 | 0.76 | 0.0033 |
| 175 | 403676.1 | 1656490 | 1.14 | 2.25 | −0.26 | 1.07 | 1.54 | 0.63 | | |
| 176 | 138472CB1 | 2373085 | 2.98 | 2.17 | 1.23 | 2.70 | 2.54 | 2.57 | 1.13 | 0.6288 |
| 177 | 253672.11 | 2851539 | −1.14 | −1.56 | −0.63 | −1.43 | −0.93 | −1.29 | −0.20 | 0.5998 |
| 178 | 201928.3 | 2126712 | −1.90 | −1.80 | −1.40 | −2.33 | −2.34 | −1.43 | | |
| 179 | 198643.1 | 2187262 | −1.48 | −1.07 | −0.20 | −1.26 | −1.07 | −0.68 | | |
| 180 | 979448.2 | 1497012 | 1.41 | 0.48 | 1.29 | 1.14 | 1.26 | 1.17 | 1.07 | 0.9824 |
| 181 | 460745.7 | 1381145 | 1.29 | 0.80 | 1.46 | 1.07 | 1.69 | 1.26 | | |
| 182 | 399300.18 | 2842978 | 1.46 | −0.07 | 1.61 | 0.43 | 1.43 | 1.07 | 2.10 | 0.4263 |
| 183 | 248091.1 | 1607083 | 1.20 | 1.03 | 0.68 | 1.20 | 1.84 | 0.92 | | |
| 184 | 4030737CB1 | 2440848 | 1.70 | 0.93 | 1.68 | 1.26 | 1.49 | 1.56 | 1.58 | 0.3354 |
| 185 | 3012575CB1 | 3012575 | 1.72 | 0.81 | 1.48 | 2.63 | 1.49 | 1.46 | 1.10 | 0.3625 |
| 186 | 221827.1 | 2057406 | 0.89 | 0.63 | 2.12 | 1.26 | 2.43 | 1.63 | | |
| 187 | 238660.5 | 1604437 | 0.63 | 2.07 | 0.89 | 1.26 | 3.06 | 1.14 | 2.17 | 0.922 |
| 188 | 2171401CB1 | 2055814 | −1.35 | −1.00 | −1.12 | −1.40 | −2.48 | −1.61 | −0.26 | 0.5691 |
| 189 | 1420940CB1 | 1603408 | 2.12 | 0.43 | 2.19 | 1.94 | 2.27 | 1.87 | | |
| 190 | 1383160.14 | 1834906 | 1.14 | 0.20 | 1.58 | 1.14 | 0.26 | 1.26 | | |
| 191 | 233331.8 | 2154725 | 1.38 | 1.04 | 1.48 | 0.48 | 1.23 | 0.98 | 1.38 | 0.8431 |
| 192 | 338217.10 | 1989545 | 1.51 | 1.14 | 2.23 | 2.42 | 1.48 | 1.00 | | |
| 193 | 5982278CB1 | 2640427 | −1.48 | −1.83 | 0.00 | −1.58 | −1.51 | −0.66 | 0.07 | 0.1445 |
| 194 | 1097030.1 | 1760232 | 1.63 | 0.13 | 1.51 | 1.87 | 1.20 | 0.87 | | |
| 195 | 1100509.4 | 2215183 | 1.96 | 0.85 | 1.80 | 1.53 | 1.63 | 1.10 | 1.38 | 0.2493 |
| 196 | 199671.1 | 1694119 | 1.85 | 1.26 | 2.02 | 1.93 | 1.58 | 1.14 | | |
| 197 | 232137.20 | 2053221 | −0.20 | −1.89 | −0.58 | −1.14 | −1.25 | −1.17 | | |
| 198 | 898608.2 | 3112520 | 1.04 | 0.92 | 1.04 | 1.20 | 1.26 | 1.43 | | |
| 199 | 411373.3 | 1625856 | 1.85 | −0.85 | 1.42 | 1.63 | 2.02 | 1.72 | | |
| 200 | 1092387.17 | 2070554 | −2.14 | −1.32 | −0.32 | −3.11 | 0.67 | −1.83 | | |
| 201 | 201906.5 | 3733666 | 1.78 | 1.68 | 1.23 | −1.20 | 0.07 | 1.26 | | |
| 202 | 3607580CB1 | 3607580 | 0.93 | 0.07 | 1.29 | 2.04 | 2.43 | 2.03 | 2.65 | 0.1241 |
| 203 | 991163CB1 | 1809178 | 1.29 | 1.43 | 1.74 | 0.46 | 2.66 | 1.29 | 0.00 | 0.3788 |
| 204 | 1309633.1 | 1880421 | −1.04 | −1.29 | −0.43 | 0.12 | −1.89 | −1.40 | | |
| 205 | 1384719.29 | 4372330 | −1.81 | −1.72 | −0.26 | −1.68 | −1.45 | −0.96 | 0.32 | 0.3135 |
| 206 | 178250.2 | 3075015 | −1.13 | −1.59 | −0.48 | −1.68 | −1.44 | −0.57 | | |
| 207 | 1099945.13 | 1911742 | −1.54 | −1.23 | −1.35 | −1.41 | −0.14 | −1.68 | | |
| 208 | 2172334CB1 | 2172334 | 2.03 | 0.85 | 1.70 | 1.61 | 1.48 | 1.80 | 0.58 | 0.673 |
| 209 | 253946.17 | 3625189 | 1.46 | 0.76 | 1.07 | 1.61 | 1.68 | 0.85 | | |
| 210 | 008513.49 | 2057510 | −6.64 | −5.91 | −4.12 | −6.61 | 0.00 | −1.38 | 0.07 | 0.0402 |
| 211 | 1092257.2 | 3440567 | 1.35 | 0.85 | 1.40 | 1.44 | 1.63 | 2.01 | 1.04 | 0.762 |
| 211 | 1092257.2 | 1447795 | 1.23 | 0.43 | 1.42 | 1.65 | 0.92 | 2.15 | | |
| 211 | 1092257.2 | 1962235 | 1.10 | 0.76 | 1.84 | 1.14 | 1.99 | 1.65 | | |
| 212 | 4349106CB1 | 2820861 | 1.35 | 0.68 | 1.77 | 1.43 | 2.05 | 2.02 | | |
| 213 | 1814803CB1 | 1698951 | −1.56 | −1.43 | −0.72 | −1.86 | −1.70 | −0.92 | | |
| 214 | 1141764.8 | 1926114 | −0.96 | −1.56 | −1.04 | −1.23 | 0.38 | −1.83 | | |
| 215 | 406531.1 | 1603580 | 1.70 | 0.81 | 1.17 | 1.26 | 1.74 | 1.07 | | |
| 216 | 429307.4 | 3144021 | −3.74 | −1.70 | −1.25 | −3.24 | 0.00 | −0.63 | 0.00 | 0.0761 |
| 217 | 239515.9 | 1675369 | 1.93 | 1.00 | 1.54 | 1.43 | 1.93 | 1.51 | 0.38 | 0.4606 |
| 218 | 1089210.1 | 5033671 | −2.70 | −1.43 | 0.43 | −4.18 | 2.15 | −2.02 | 0.43 | 0.8466 |
| 219 | 010796.18 | 1445895 | 1.82 | 0.85 | 1.43 | 1.14 | 1.35 | 1.35 | | |
| 220 | 3752346CB1 | 1378037 | −1.51 | −1.07 | −0.48 | −1.43 | −1.92 | −0.20 | | |
| 221 | 2729828CB1 | 1858171 | −1.86 | −1.23 | −3.09 | −3.35 | 1.13 | −0.48 | | |
| 222 | 1648579CB1 | 3142624 | 1.35 | 0.07 | 1.74 | 2.32 | 2.58 | 1.10 | | |
| 223 | 1454418CB1 | 1468660 | −1.63 | −2.26 | −0.53 | −2.14 | −3.14 | −0.38 | 0.37 | 0.0305 |
| 224 | 995529.10 | 2957476 | −1.80 | −2.90 | −0.50 | −3.55 | −3.67 | −1.10 | | |
| 225 | 2126751CB1 | 2126751 | −1.17 | −1.56 | −0.63 | −1.48 | −1.10 | −1.29 | −0.14 | 0.5579 |
| 226 | 569648CB1 | 2720467 | 2.10 | 2.02 | 1.20 | 1.71 | 2.26 | 1.98 | 0.93 | 0.5797 |
| 227 | 1352789CB1 | 3000146 | 2.26 | 1.48 | 1.29 | 1.86 | 1.38 | 0.68 | 1.35 | 0.2624 |
| 228 | 1635966CB1 | 2046165 | 1.56 | 1.94 | 1.23 | 1.13 | −0.85 | 0.93 | 1.63 | 0.6637 |
| 229 | 349415.6 | 3074415 | −2.14 | 0.19 | −0.72 | −1.51 | −2.79 | −2.91 | | |
| 230 | 406438.14 | 2797787 | −1.04 | −1.26 | −0.80 | −1.26 | −0.72 | −1.49 | | |
| 231 | 1471808CB1 | 3074415 | −2.14 | 0.19 | −0.72 | −1.51 | −2.79 | −2.91 | | |
| 232 | 245595.1 | 2052480 | −1.43 | −2.10 | 0.00 | −0.69 | −2.42 | −1.68 | | |
| 233 | 2784232CB1 | 3151158 | 1.43 | 1.17 | 1.10 | 1.54 | 1.48 | 0.26 | | |
| 234 | 2742913CB1 | 2516950 | 1.03 | 0.32 | 1.04 | 1.10 | 0.81 | 1.22 | 1.48 | 0.0717 |
| 235 | 1509972CB1 | 4047785 | 1.40 | 1.96 | 1.00 | 1.25 | 1.26 | 1.65 | −0.07 | 0.6192 |
| 236 | 1097638.1 | 1903760 | 0.14 | 0.38 | 1.89 | 1.61 | 2.74 | 1.26 | | |
| 237 | 900035.58 | 5186773 | −1.98 | −1.07 | −1.74 | −2.91 | −0.96 | −1.91 | −0.53 | 0.5926 |
| 237 | 900035.58 | 1900306 | −3.13 | −1.10 | −2.77 | −2.47 | −0.68 | −2.47 | | |
| 238 | 1075592.6 | 1423848 | −1.26 | −0.32 | −1.86 | −1.94 | −1.35 | −1.48 | −0.85 | 0.7018 |
| 239 | 984540.1 | 1393337 | 2.00 | 0.20 | 2.00 | 1.29 | 2.09 | 1.39 | 1.14 | 0.54 |
| 240 | 378633.40 | 1821938 | 1.29 | 1.45 | 1.14 | 1.89 | 2.79 | 1.46 | | |
| 241 | 477387.7 | 3100048 | −1.00 | −0.53 | −1.40 | −1.74 | −1.67 | −1.48 | | |
| 242 | 347049.9 | 2057158 | 2.19 | 1.87 | 1.72 | 1.92 | 2.49 | 1.14 | 0.88 | 0.0088 |
| 243 | 1097717.18 | 5065213 | −1.32 | −2.15 | −0.58 | −1.56 | −1.49 | −0.58 | 0.63 | 0.214 |
| 244 | 2832214CB1 | 2832214 | −1.23 | −2.05 | −0.85 | −1.07 | −1.22 | −0.43 | | |
| 245 | 2947513CB1 | 5984038 | −1.32 | −2.28 | −0.76 | −1.54 | −1.48 | −0.63 | 0.53 | 0.2065 |
| 246 | 1655369CB1 | 2056840 | 1.17 | 1.03 | 1.29 | 1.26 | 1.56 | 1.00 | 0.85 | 0.0456 |
| 247 | 039170.3 | 2418484 | −1.37 | −2.28 | −0.76 | −1.72 | −2.14 | −0.85 | | |
| 248 | 1074926.1 | 2947513 | −1.43 | −2.33 | −0.96 | −1.54 | −1.37 | −0.63 | | |
| 249 | 344297.5 | 62144 | −1.03 | −1.06 | 0.20 | −1.12 | −1.63 | −0.42 | 0.00 | 0.1143 |
| 250 | 410721.1 | 4688 | −2.23 | −0.07 | −2.96 | −1.51 | −3.54 | −2.63 | | |
| 251 | 1121097.1 | 1450641 | −1.43 | 0.79 | −1.74 | −1.10 | 0.53 | −1.45 | | |
| 252 | 1097450.22 | 3374194 | 1.38 | 1.20 | 0.38 | 1.51 | 1.45 | 0.20 | | |
| 253 | 238533.1 | 2790947 | 1.96 | 0.31 | 1.91 | 1.33 | 1.83 | 0.92 | | |
| 254 | 1097080.8 | 2694381 | 0.58 | 0.32 | 1.65 | 1.14 | 1.48 | 1.04 | | |
| 255 | 1097450.20 | 3374194 | 1.38 | 1.20 | 0.38 | 1.51 | 1.45 | 0.20 | | |
| 256 | 2514988CB1 | 1886886 | 1.46 | −0.14 | 2.06 | 1.86 | 2.15 | 1.10 | 2.12 | 0.8568 |
| 257 | 3130485CB1 | 1987238 | 1.77 | 2.74 | 2.55 | 1.26 | 3.19 | 0.53 | | |
| 258 | 1330234.11 | 5392723 | −2.05 | −1.22 | −2.23 | −2.29 | −1.38 | −1.52 | | |
| 259 | 1943624CB1 | 1943624 | 0.37 | 1.45 | 1.28 | 2.72 | 1.93 | 0.63 | −1.26 | 0.2657 |
| 260 | 2189816CB1 | 2189816 | 1.79 | 1.29 | 0.60 | 2.36 | 1.29 | 2.01 | 1.00 | 0.9536 |
| 261 | 039027.1 | 3861522 | −2.17 | −2.35 | −1.41 | −1.63 | −0.46 | −1.20 | | |
| 262 | 2502336CB1 | 2507719 | −1.65 | −0.07 | −2.72 | −0.99 | −2.77 | −1.85 | | |
| 263 | 3592543CB1 | 5066393 | 1.20 | 0.48 | 1.83 | 2.17 | 0.92 | 1.85 | | |
| 264 | 399589.1 | 2070856 | 1.68 | 0.96 | 1.58 | 1.71 | 1.96 | 0.96 | | |
| 265 | 2175401CB1 | 1833174 | 1.48 | 1.10 | 0.92 | 1.38 | 1.38 | 0.26 | | |
| 266 | 084455.9 | 1356268 | 1.17 | 1.17 | 2.17 | 2.32 | 2.28 | 1.23 | | |
| 267 | 233041.5 | 2060622 | −1.40 | −1.20 | −0.14 | −1.77 | 1.15 | −0.81 | | |
| 268 | 1382961.5 | 3184882 | −4.32 | −1.91 | −1.58 | −3.76 | 0.58 | −0.20 | 0.37 | 0.0581 |
| 269 | 1518310CB1 | 1518310 | −3.23 | −2.77 | −1.06 | −2.69 | 0.00 | −1.54 | 0.37 | 0.3569 |
| 270 | 1517817CB1 | 1402078 | 2.14 | 1.04 | 0.38 | 1.79 | 1.54 | 0.00 | 1.05 | 0.3119 |
| 271 | 3090387CB1 | 1714684 | 2.08 | 1.23 | 0.96 | 1.65 | 1.78 | 1.00 | 1.14 | 0.0793 |
| 272 | 3836893CB1 | 3836893 | 2.60 | 0.68 | 1.24 | 3.56 | 2.49 | 2.26 | 1.29 | 0.6605 |
| 273 | 394121.2 | 2796118 | −1.60 | 1.14 | 1.40 | 1.50 | 2.03 | 1.10 | −0.53 | 0.6076 |
| 274 | 2347046CB1 | 2347046 | −1.26 | −1.70 | −0.63 | −1.74 | −2.76 | −1.29 | 0.07 | 0.3472 |
| 275 | 1137894.1 | 2059420 | 1.46 | 0.80 | 2.74 | 4.02 | 3.71 | 1.54 | 0.07 | 0.1819 |
| 276 | 1137536.4 | 1753015 | −1.35 | −1.53 | −0.48 | −1.77 | −1.87 | −1.29 | | |
| 277 | 2501808CB1 | 2501808 | −1.17 | −1.29 | −0.89 | −2.67 | −2.90 | −1.10 | −0.38 | 0.1348 |
| 278 | 4767318CB1 | 3088261 | −1.07 | −1.87 | −0.26 | −1.87 | −1.32 | 0.20 | −0.14 | 0.0128 |
| 279 | 1223705CB1 | 1633118 | −3.41 | −0.07 | −1.17 | −3.60 | 0.13 | −2.76 | −0.53 | 0.9891 |
| 280 | 296696.7 | 851875 | 1.23 | 0.57 | 1.38 | 2.29 | 1.20 | 0.93 | 1.23 | 0.4562 |
| 281 | 410320.1 | 2886536 | 1.81 | 1.81 | 1.07 | 2.25 | 2.12 | 0.58 | | |
| 282 | 426109.1 | 2057823 | −2.03 | −2.53 | −0.37 | −2.64 | −2.57 | −1.17 | 0.07 | 0.1849 |
| 283 | 1098589.27 | 2152363 | −1.87 | −1.83 | −1.14 | −1.79 | −2.66 | −1.07 | | |
| 284 | 233575.1 | 1222942 | −0.48 | −1.35 | −0.72 | −1.45 | −1.53 | −1.49 | | |
| 285 | 235191.4 | 1997915 | −1.35 | −1.94 | −1.23 | −3.06 | −2.97 | −1.43 | −0.43 | 0.1838 |
| 286 | 468221.19 | 1662856 | −1.34 | −1.26 | −1.17 | −1.77 | −0.84 | −0.43 | | |
| 287 | 986342.1 | 2811372 | 0.96 | 0.76 | 1.38 | 1.61 | 1.07 | 1.17 | | |
| 288 | 240120.3 | 2795141 | 1.26 | 0.20 | 2.48 | 2.09 | 2.56 | 1.98 | 1.32 | 0.9076 |
| 289 | 5511889CB1 | 6105902 | 0.07 | 0.26 | 1.92 | 2.42 | 2.05 | 1.04 | | |
| 290 | 3009578CB1 | 3009578 | −1.07 | −1.10 | −0.43 | −1.43 | −1.83 | −0.68 | | |
| 291 | 982520.1 | 4821815 | −3.52 | −0.53 | −1.16 | −2.86 | −0.20 | −2.53 | −2.23 | 0.3352 |
| 292 | 899156.36 | 2851850 | 1.07 | 0.48 | 0.92 | 1.63 | 1.79 | 1.41 | | |
| 293 | 1748428CB1 | 1658215 | 1.70 | 0.07 | 0.99 | 1.53 | 1.92 | 1.07 | 1.51 | 0.9089 |
| 294 | 238540.1 | 1608244 | −1.07 | 1.38 | 1.14 | 0.89 | 1.70 | 1.04 | | |
| 295 | 898495.3 | 5261507 | −0.96 | −1.77 | −0.48 | −1.40 | −1.22 | −1.10 | | |
| 296 | 369213.48 | 1292449 | 1.17 | 1.54 | 0.20 | 2.07 | 1.30 | 0.43 | | |
| 297 | 3084563CB1 | 5834427 | 1.38 | 0.00 | 1.41 | 1.35 | 1.54 | 0.93 | | |
| 298 | 237613.7 | 1975550 | 2.00 | 0.81 | 2.16 | 1.83 | 1.54 | 1.53 | 0.07 | 0.4332 |
| 299 | 2630652CB1 | 2152363 | −1.87 | −1.83 | −1.14 | −1.79 | −2.66 | −1.07 | | |
| 300 | 5785224CB1 | 1212335 | −1.49 | −1.29 | −1.10 | −0.81 | −0.48 | −1.29 | | |
| 301 | 409911.8 | 2219364 | 2.33 | 1.53 | 1.95 | 1.13 | 1.91 | 1.78 | 1.20 | 0.5089 |
| 302 | 2418523CB1 | 2454639 | −0.13 | −1.32 | 0.32 | −1.74 | −2.11 | −0.07 | 1.14 | 0.1642 |
| 303 | 1091297.30 | 2418490 | 1.14 | 1.14 | 1.14 | 1.53 | 1.07 | −0.68 | 0.26 | 0.1952 |
| 304 | 369213.2 | 2200842 | 1.61 | 2.79 | 1.04 | 2.14 | 1.14 | −0.32 | −0.38 | 0.0029 |
| 305 | 221869.5 | 5523515 | 1.38 | 0.38 | 1.41 | 1.74 | 0.46 | 1.92 | | |
| 306 | 995529.6 | 2957476 | −1.80 | −2.90 | −0.50 | −3.55 | −3.67 | −1.10 | | |
| 307 | 982959.1 | 3115823 | −2.36 | −0.76 | −1.23 | −2.17 | −0.48 | −1.87 | −1.00 | 0.9556 |
| 308 | 342074.1 | 4408943 | 2.51 | 0.00 | 0.53 | 1.74 | 2.32 | −1.06 | 1.63 | 0.5484 |
| 309 | 245194.2 | 1512826 | −1.29 | −0.43 | −1.06 | −1.89 | −0.24 | −1.51 | −1.48 | 0.1607 |
| 310 | 246444.6 | 3382391 | −3.48 | −1.94 | −1.24 | −6.57 | 0.13 | −0.32 | | |
| 311 | 374054.4 | 3843227 | 1.23 | 0.99 | 1.20 | 2.18 | 1.74 | 1.07 | 1.61 | 0.7411 |
| 312 | 001322.4 | 2663164 | 1.51 | 0.20 | 1.07 | 1.20 | 1.23 | 0.63 | 1.04 | 0.5361 |
| 313 | 406438.12 | 2797787 | −1.04 | −1.26 | −0.80 | −1.26 | −0.72 | −1.49 | | |
| 314 | 1382961.12 | 4382348 | −5.07 | −4.05 | −3.81 | −5.39 | 1.14 | −0.13 | | |
| 315 | 1359783CB1 | 3138721 | 1.51 | −1.00 | 0.81 | 1.34 | 2.49 | 1.40 | | |
| 316 | 1796035CB1 | 3986547 | 2.31 | 1.77 | 1.29 | 1.85 | 2.18 | 1.72 | | |
| 317 | 444850.11 | 2860704 | 1.68 | 1.10 | 1.14 | 1.14 | 0.72 | −0.63 | | |
| 318 | 236062CB1 | 2238411 | 1.14 | 0.20 | 1.65 | 2.39 | 1.63 | 0.53 | 1.29 | 0.4169 |
| 319 | 333238.12 | 2833637 | −1.10 | −1.26 | −0.14 | −1.48 | −1.14 | −1.17 | | |
| 320 | 475547.2 | 1955363 | 1.83 | 1.40 | 1.63 | 1.63 | 2.92 | 1.23 | | |
| 321 | 977929.1 | 4112181 | 1.26 | 0.72 | 1.14 | 1.86 | 1.51 | 1.26 | | |
| 322 | 1362715CB1 | 1816113 | 1.79 | 1.26 | 2.15 | 2.94 | 2.46 | 1.46 | 0.63 | 0.1629 |
| 323 | 3117642CB1 | 3117642 | −2.23 | 1.56 | −2.72 | −1.54 | −0.14 | −2.45 | | |
| 324 | 2026270CB1 | 2026270 | −4.45 | −1.71 | −1.77 | −3.14 | 0.00 | −0.34 | 0.00 | 0.0512 |
| 325 | 981662.2 | 4401727 | −1.10 | −1.22 | −0.72 | −1.98 | −1.60 | −0.53 | | |
| 326 | 453004.10 | 2394888 | 1.58 | 1.46 | 1.07 | 1.96 | 1.85 | 1.35 | 0.38 | 0.3609 |
| 327 | 978147.7 | 2784394 | −1.10 | −1.17 | −0.68 | −1.98 | −3.04 | −0.43 | | |
| 328 | 2132626CB1 | 541875 | 1.80 | 0.85 | 2.23 | 2.33 | 1.96 | 1.29 | 2.55 | 0.9157 |
| 329 | 402716.37 | 4199466 | 1.74 | 1.23 | 1.16 | 3.08 | 1.85 | 1.93 | 1.41 | 0.7577 |
| 330 | 464482.1 | 625374 | 1.82 | 2.05 | 1.46 | 0.53 | 0.14 | 1.56 | 1.94 | 0.2439 |
| 331 | 402716.20 | 4199466 | 1.74 | 1.23 | 1.16 | 3.08 | 1.85 | 1.93 | 1.41 | 0.7577 |
| 332 | 1137710.5 | 1713191 | 1.23 | 1.00 | 1.14 | 1.64 | −0.07 | 1.09 | 1.20 | 0.6157 |
| 333 | 348912.3 | 1716655 | 2.70 | 2.31 | 1.80 | 1.65 | 2.87 | 0.43 | −1.92 | 0.2253 |
| 334 | 474926.11 | 2512203 | −2.26 | −1.87 | −1.29 | −2.54 | −1.93 | −0.84 | 0.00 | 0.1128 |
| 335 | 406804.4 | 3130454 | 2.03 | 0.58 | 2.65 | 1.63 | 1.70 | 2.05 | | |
| 336 | 480855.1 | 3234063 | 1.61 | 1.70 | 1.17 | 2.17 | 2.07 | 0.63 | | |
| 337 | 238593.5 | 211779 | −2.17 | 1.19 | −1.94 | −1.04 | 0.20 | −2.63 | | |
| 338 | 373514.7 | 4740251 | −1.89 | 2.29 | −2.76 | −1.35 | 0.63 | −2.22 | | |
| 339 | 1383354.10 | 5057204 | 0.89 | 0.38 | 1.58 | 1.41 | 2.01 | 1.26 | | |
| 340 | 3120070CB1 | 3120070 | −3.43 | −2.34 | −1.65 | −3.95 | −2.15 | −0.58 | −0.48 | 0.0067 |
| 341 | 253987.19 | 2232658 | 1.26 | −0.38 | 1.07 | 0.76 | 2.17 | 1.51 | 1.68 | 0.2104 |
| 342 | 133425.16 | 3510656 | 1.29 | 1.29 | 0.72 | 2.03 | 1.74 | 1.32 | 1.00 | 0.4025 |
| 343 | 468221.18 | 1662856 | −1.34 | −1.26 | −1.17 | −1.77 | −0.84 | −0.43 | | |
| 344 | 020093.8 | 2102756 | 2.47 | 1.78 | 0.68 | 2.17 | 1.77 | 0.48 | 2.09 | 0.6523 |
| 345 | 1556751CB1 | 1986121 | 1.35 | 0.96 | 1.35 | 1.65 | 1.49 | 1.10 | 0.96 | 0.0587 |
| 346 | 1397976.1 | 4628258 | 1.74 | 1.34 | 1.92 | 1.19 | 2.28 | 0.26 | | |
| 347 | 233828.16 | 1362831 | 1.68 | 0.43 | 1.82 | 1.92 | 2.13 | 1.29 | 2.15 | 0.8343 |
| 348 | 1253414CB1 | 5681633 | 1.07 | 0.48 | 1.59 | 1.74 | 1.74 | 1.23 | | |
| 349 | 1101068.1 | 5856402 | 1.74 | 0.77 | 2.36 | 1.45 | 1.72 | 1.26 | | |
| 350 | 006922.1 | 2934515 | 1.17 | 0.07 | 1.91 | 0.89 | 1.06 | 1.06 | | |
| 351 | 333398.5 | 2456903 | 1.96 | 0.26 | 1.91 | 1.28 | 1.87 | 1.26 | 1.10 | 0.4498 |
| 352 | 235725.21 | 2095728 | −1.74 | −2.17 | 0.07 | −1.65 | −1.20 | −0.68 | 0.53 | 0.2272 |
| 353 | 242472.14 | 4572916 | −2.25 | −2.90 | −1.84 | −1.13 | 0.14 | −1.00 | | |
| 354 | 253550.20 | 3397390 | −1.14 | 0.88 | −1.74 | −2.55 | −0.43 | −2.02 | −1.37 | 0.3441 |
| 355 | 216262.3 | 3813934 | −0.20 | −5.50 | −1.32 | 2.03 | −0.63 | −2.91 | −1.17 | 0.5725 |
| 356 | 235191.3 | 1997915 | −1.35 | −1.94 | −1.23 | −3.06 | −2.97 | −1.43 | −0.43 | 0.1838 |
| 357 | 480337.45 | 4602215 | −1.38 | −2.02 | −0.63 | −3.10 | −2.00 | −1.26 | | |
| 358 | 199939.6 | 1858415 | −1.46 | −1.87 | −0.68 | −1.65 | −2.54 | −0.57 | 0.51 | 0.1443 |
| 359 | 201204.9 | 4088394 | −1.56 | −2.09 | −0.37 | −0.63 | −1.91 | −1.07 | −0.07 | 0.3432 |
| 360 | 201887.2 | 3478024 | −1.41 | −1.14 | −0.26 | −1.59 | −1.35 | −0.72 | | |
| 361 | 1136056.1 | 3527982 | 1.10 | 1.35 | 2.88 | 5.45 | 4.68 | 2.17 | 0.85 | 0.2166 |
| 362 | 994977.1 | 3722056 | −2.50 | −0.72 | −2.24 | −2.57 | 0.26 | −2.22 | −0.68 | 0.9224 |
| 363 | 888669.8 | 4721466 | −1.29 | −1.23 | −0.20 | −2.36 | −1.74 | −0.37 | | |
| 364 | 345860.20 | 5160686 | 2.00 | 1.79 | 1.04 | 2.10 | 2.36 | 1.29 | | |
| 365 | 480337.43 | 1459082 | −1.29 | −1.70 | −0.53 | −2.87 | −1.65 | −1.23 | −0.14 | 0.2945 |
| 365 | 480337.43 | 4602215 | −1.38 | −2.02 | −0.63 | −3.10 | −2.00 | −1.26 | | |
| 366 | 399300.14 | 2842978 | 1.46 | −0.07 | 1.61 | 0.43 | 1.43 | 1.07 | 2.10 | 0.4263 |
| 367 | 368015.2 | 3115792 | −1.04 | −2.43 | −1.68 | −0.43 | 0.85 | −1.17 | | |
| 368 | 227550.1 | 3771020 | −1.85 | 1.56 | −2.42 | −1.56 | 0.43 | −2.03 | | |
| 369 | 201906.6 | 3733666 | 1.78 | 1.68 | 1.23 | −1.20 | 0.07 | 1.26 | | |
| 370 | 349589.10 | 127321 | −2.18 | 2.30 | −2.60 | −1.32 | 0.14 | −2.89 | −1.61 | 0.2318 |
| 371 | 3713867CB1 | 2061401 | 2.17 | 1.51 | 1.43 | 1.56 | 1.94 | 0.14 | | |
| 372 | 235943.27 | 2957567 | 2.34 | 2.70 | 1.79 | 2.79 | 3.08 | 1.13 | −0.14 | 0.16 |
| 373 | 241742.1 | 1295905 | 0.85 | 0.00 | 1.17 | 1.35 | 1.68 | 1.23 | | |
| 374 | 333680.1 | 3138456 | 1.79 | 1.00 | 1.32 | 0.69 | 1.68 | 1.17 | 1.70 | 0.7134 |
| 375 | 411429.3 | 1424886 | −1.70 | −3.09 | −0.38 | −2.13 | −1.00 | −1.20 | | |
| 376 | 2356055CB1 | 2356055 | 1.58 | 0.58 | 1.65 | 1.94 | 1.26 | 1.69 | 0.46 | 0.69 |
| 377 | 239579.3 | 1704713 | 1.29 | 0.53 | 1.22 | 1.09 | 1.35 | 0.42 | 1.72 | 0.9747 |
| 378 | 332240.1 | 2201507 | −2.12 | −2.89 | −1.07 | −2.36 | −2.10 | −1.72 | | |
| 379 | 255161.1 | 2270986 | 1.32 | 1.82 | 0.20 | 1.29 | 1.32 | 0.85 | 1.49 | 0.9652 |
| 380 | 2454384CB1 | 2454384 | 2.24 | −0.53 | 1.93 | 0.96 | 1.17 | 1.83 | | |
| 381 | 1383298.3 | 1510539 | 1.86 | 1.04 | 0.63 | 0.77 | 2.45 | 1.22 | 2.22 | 0.6032 |
| 382 | 383376.7 | 5551761 | 1.70 | 0.76 | 1.29 | 2.25 | 1.60 | 1.14 | 0.38 | 0.2398 |
| 383 | 230816.1 | 1526322 | −1.94 | 1.54 | −2.32 | −1.41 | 0.20 | −2.14 | | |
| 384 | 211949.2 | 1966295 | 1.50 | 0.58 | 1.35 | 1.14 | 1.55 | 0.77 | | |
| 385 | 238853.42 | 1450886 | −2.03 | −1.43 | −2.12 | −3.00 | 1.83 | −1.14 | | |
| 386 | 333165.2 | 1682337 | 1.38 | 1.26 | 0.76 | 1.14 | 1.65 | 0.00 | | |
| 387 | 344868.12 | 3333118 | −0.48 | −1.04 | −1.00 | −1.07 | −1.51 | −1.23 | | |
| 388 | 148304.14 | 3646303 | 2.28 | 1.35 | 1.87 | 1.16 | 1.18 | 1.14 | | |
| 389 | 247747.6 | 3124204 | −1.70 | −2.25 | −0.14 | −1.80 | −1.98 | −0.58 | | |
| 390 | 001153.12 | 5546984 | 1.26 | 0.72 | 1.46 | 1.17 | −1.14 | 0.68 | 1.72 | 0.5264 |
| 391 | 1095728.19 | 414480 | −1.69 | −2.96 | −0.63 | −1.04 | 0.43 | −2.08 | −0.81 | 0.7753 |
| 392 | 222604.7 | 2745735 | −1.51 | −1.10 | −0.89 | −2.36 | −1.98 | −1.00 | | |
| 393 | 1094984.14 | 2620487 | −1.14 | −2.80 | −1.26 | −2.07 | 0.26 | −1.07 | | |
| 394 | 400702.1 | 1969974 | 1.94 | 0.67 | 1.35 | 1.45 | 0.63 | 2.02 | | |
| 395 | 196557.1 | 1927026 | 0.88 | 0.58 | 1.83 | 1.35 | 1.83 | 1.20 | | |
| 396 | 237208.5 | 993365 | −1.70 | −1.72 | −0.48 | −2.12 | −1.31 | −0.38 | | |
| 397 | 337792.2 | 1832594 | −1.63 | −1.92 | −0.43 | −1.53 | −1.35 | −1.04 | | |
| 398 | 398970.3 | 4885619 | 2.24 | 1.78 | 1.07 | 0.87 | 1.10 | 0.94 | 1.38 | 0.5002 |
| 399 | 1309633.3 | 1880421 | −1.04 | −1.29 | −0.43 | 0.12 | −1.89 | −1.40 | | |
| 400 | 981662.1 | 4401727 | −1.10 | −1.22 | −0.72 | −1.98 | −1.60 | −0.53 | | |
| 401 | 081187.1 | 2416447 | −0.77 | −1.38 | −0.63 | −1.61 | −3.32 | −1.20 | −1.59 | 0.7885 |
| 402 | 086533.6 | 219839 | 1.20 | 0.48 | 1.56 | 2.59 | 1.59 | 1.23 | 0.53 | 0.3019 |
| 403 | 1093481.2 | 1962139 | 1.41 | 0.53 | 1.92 | 1.87 | 2.46 | 1.38 | | |
| 404 | 024494.7 | 1938744 | 1.20 | 0.42 | 1.54 | 1.26 | 1.68 | 1.07 | | |
| 405 | 888669.7 | 4721466 | −1.29 | −1.23 | −0.20 | −2.36 | −1.74 | −0.37 | | |
| 406 | 428206.1 | 2675284 | 1.77 | 0.76 | 1.87 | 0.76 | 1.42 | 1.74 | | |
| 407 | 1328026.7 | 549728 | −5.13 | −1.58 | −2.00 | −5.75 | 0.00 | −0.14 | | |
| 408 | 475113.7 | 1700462 | −1.17 | −1.20 | −0.88 | −1.26 | −1.85 | −0.53 | | |
| 409 | 204392.5 | 2950394 | 2.15 | 1.17 | 0.63 | 0.92 | 1.65 | 1.06 | 1.29 | 0.6786 |
| 410 | 1092257.12 | 3440567 | 1.35 | 0.85 | 1.40 | 1.44 | 1.63 | 2.01 | 1.04 | 0.762 |
| 411 | 1134990.3 | 3878116 | 2.72 | 1.07 | 1.58 | 1.83 | 1.93 | 1.56 | 1.26 | 0.2347 |
| 412 | 1095223.13 | 4158982 | −1.10 | −1.83 | −0.38 | −1.43 | −2.51 | −1.14 | | |
| 413 | 214654.1 | 1681727 | 1.96 | 0.58 | 1.56 | 0.63 | 1.67 | 1.14 | | |
| 414 | 086533.5 | 219839 | 1.20 | 0.48 | 1.56 | 2.59 | 1.59 | 1.23 | 0.53 | 0.3019 |
| 415 | 474117.5 | 3992320 | −1.35 | −1.40 | −0.43 | −1.43 | −1.29 | −0.88 | 0.13 | 0.3335 |
| 416 | 410812.1 | 3380665 | −1.54 | −1.17 | −1.46 | −0.96 | −0.48 | −1.98 | | |
| 417 | 011822.2 | 2887646 | −1.26 | −2.43 | −0.81 | −1.43 | −1.38 | −0.58 | | |
| 418 | 252899.7 | 2811710 | 1.58 | 0.58 | 1.85 | 1.74 | 1.65 | 1.41 | | |
| 419 | 1511488CB1 | 1511488 | 1.96 | 0.76 | 0.94 | 1.03 | 1.90 | 1.58 | | |
| 420 | 1383797.1 | 1466844 | −3.12 | 2.44 | −4.14 | −1.82 | 0.00 | −4.01 | | |
| 421 | 400745.1 | 4205017 | 1.93 | 1.68 | 1.46 | 1.20 | 0.92 | 1.29 | | |
| 422 | 029618.1 | 1223877 | −1.14 | −1.13 | −0.51 | −1.51 | −1.15 | −0.77 | | |
| 423 | 245722.8 | 2508079 | −1.46 | −1.43 | 0.48 | −2.07 | 2.09 | 0.43 | | |
| 424 | 981488.1 | 2159213 | 1.56 | 0.58 | 1.56 | 1.63 | 0.77 | 1.09 | | |
| 424 | 981488.1 | 2449016 | 1.32 | 0.13 | 1.17 | 1.56 | 1.56 | 1.07 | | |
| 425 | 241858.1 | 4460483 | 1.70 | 1.35 | 1.23 | 1.78 | 1.38 | 1.34 | | |
| 426 | 1466276CB1 | 739191 | −1.60 | −1.31 | −0.48 | −2.52 | −2.25 | −0.72 | 0.81 | 0.2291 |
| 427 | 999386.3 | 5286390 | 1.90 | 1.10 | 1.89 | 1.34 | 1.93 | 1.20 | 1.94 | 0.8975 |
| 428 | 1091079.8 | 1503624 | 1.70 | 0.53 | 1.94 | 2.29 | 2.27 | 1.78 | | |
| 429 | 407084.1 | 3423374 | 1.85 | 1.59 | 1.23 | 2.68 | 1.58 | 1.43 | 0.63 | 0.2599 |
| 430 | 215642.2 | 1923709 | −1.26 | −1.03 | −0.95 | −1.70 | 0.00 | −1.86 | | |
| 431 | 1159769.1 | 3929017 | 1.87 | 0.89 | 1.26 | −0.32 | 2.72 | 1.54 | | |
| 432 | 482336.31 | 4104390 | 1.51 | 1.31 | 1.35 | 1.62 | 1.46 | −0.38 | 0.43 | 0.1705 |
| 433 | 064703.1 | 1900111 | −2.26 | −1.87 | −1.63 | −1.77 | −0.63 | −2.61 | | |
| 434 | 294837.1 | 3255489 | −1.93 | 1.48 | 1.07 | 0.63 | 1.56 | 0.72 | | |
| 435 | 982699.1 | 5394922 | −1.91 | −1.06 | −2.00 | −2.51 | −0.99 | −1.56 | | |
| 436 | 015126.1 | 4361169 | −1.76 | −1.69 | −1.23 | −2.62 | −0.07 | 0.07 | | |
| 437 | 235132.10 | 5682290 | −1.79 | −2.64 | −2.93 | −3.24 | −2.51 | −1.32 | | |
| 438 | 1070092.1 | 2965648 | −1.41 | 1.07 | 1.23 | 1.14 | 1.60 | 0.93 | | |
| 439 | 272599.1 | 2278772 | −0.20 | −1.46 | −1.72 | −1.37 | −1.64 | −2.07 | | |
| 440 | 415901.1 | 2998131 | −1.70 | 0.95 | −1.89 | −1.46 | 0.07 | −1.85 | | |
| 441 | 477387.3 | 3100048 | −1.00 | −0.53 | −1.40 | −1.74 | −1.67 | −1.48 | | |
| 442 | 208702.1 | 3658444 | 1.23 | 1.07 | 1.29 | 0.53 | 2.48 | 0.63 | | |
| 443 | 1135039.1 | 899118 | 2.23 | 2.12 | 2.20 | 3.04 | 3.20 | 1.10 | | |
| 444 | 232649.2 | 2234640 | 1.45 | 1.07 | 1.59 | 0.38 | 1.17 | 1.70 | | |
| 445 | 340450.1 | 4639995 | −1.72 | −1.23 | −0.38 | −2.45 | −1.23 | −0.48 | | |
| 446 | 320660.1 | 5034026 | 1.51 | 1.16 | 1.26 | 1.79 | 2.02 | 1.51 | | |
| 447 | 467104.21 | 2054055 | −1.80 | −1.87 | −1.37 | −1.74 | 0.06 | −0.07 | | |
| 448 | 332382.1 | 3999569 | −1.26 | −1.28 | −0.77 | −2.66 | −0.81 | 0.20 | | |
| 449 | 2104530CB1 | 2104530 | −1.20 | −1.41 | −0.48 | −1.51 | −1.07 | −1.20 | | |
| 450 | 007228.1 | 60306 | 1.79 | 0.68 | 1.80 | 1.30 | 1.70 | 1.68 | | |
| 451 | 008098.1 | 313312 | 1.23 | 2.64 | 0.00 | 0.00 | 2.00 | 1.56 | | |
| 452 | 016124.2 | 3732960 | 1.77 | 2.29 | 0.26 | 1.89 | 1.89 | 1.48 | | |
| 453 | 016149.1 | 3736058 | −2.38 | −3.19 | 0.37 | 0.68 | −0.72 | −1.51 | | |
| 454 | 025731.1 | 2586554 | 1.48 | −0.96 | 0.63 | 1.04 | 2.46 | 1.23 | | |
| 455 | 026856.1 | 1300730 | −1.32 | 0.91 | −2.24 | −1.29 | 0.20 | −1.38 | | |
| 456 | 028918.1 | 2800380 | 1.23 | 0.87 | 1.07 | 1.53 | 1.92 | 0.14 | | |
| 457 | 032481.1 | 4073339 | −1.85 | −1.75 | 0.00 | −2.63 | −2.26 | −0.58 | | |
| 458 | 045630.1 | 1269876 | −1.98 | −0.74 | −1.58 | −1.46 | −0.85 | −1.94 | | |
| 459 | 047533.1 | 4276910 | −1.85 | −1.77 | −1.17 | −2.47 | −0.13 | −2.12 | | |
| 460 | 072540.1 | 2152929 | 1.46 | 0.81 | 1.40 | 0.93 | 1.07 | 1.54 | | |
| 461 | 076580.1 | 2264967 | −1.54 | −1.51 | −1.20 | −1.56 | 0.06 | −2.03 | | |
| 462 | 1072776.1 | 5505238 | −1.20 | −3.29 | −1.14 | −2.00 | 0.26 | −1.26 | | |
| 463 | 1100233.1 | 3725970 | −2.40 | 1.94 | −2.85 | −1.43 | 0.26 | −2.72 | | |
| 464 | 1102220.2 | 1299351 | 1.10 | −1.23 | 1.00 | 1.58 | 1.08 | −0.14 | | |
| 465 | 1328508.1 | 2669974 | 1.23 | 0.42 | 1.20 | 1.41 | 1.49 | 0.76 | | |
| 466 | 1383457.1 | 5024278 | −1.61 | −0.13 | −2.25 | −1.58 | −1.10 | −1.07 | | |
| 467 | 140230.5 | 1211835 | 1.60 | 0.43 | 2.17 | 1.60 | 2.41 | 0.88 | | |
| 468 | 150868.1 | 491129 | −1.40 | −0.07 | 0.34 | −2.55 | −1.07 | −0.63 | | |
| 469 | 172582.1 | 5622463 | 1.59 | 0.92 | 1.73 | 1.43 | 1.41 | 1.29 | | |
| 470 | 175536.1 | 122943 | 1.67 | −0.42 | 2.02 | 1.59 | 1.98 | 1.12 | | |
| 470 | 175536.1 | 5187276 | 3.17 | 0.37 | 2.63 | 2.17 | 2.40 | 2.38 | | |
| 471 | 176928.1 | 2782090 | −1.26 | −1.72 | −1.15 | −1.96 | −2.09 | −1.46 | | |
| 472 | 197252.1 | 2656358 | −2.56 | −1.89 | −1.41 | −2.86 | −0.31 | −0.85 | | |
| 473 | 1975480CB1 | 3852527 | 1.20 | 0.89 | 1.92 | 1.46 | 1.68 | 1.14 | | |
| 474 | 200578.1 | 473724 | −2.91 | −1.29 | −0.13 | −3.01 | 0.00 | −3.35 | | |
| 475 | 202117.4 | 1402988 | 2.18 | 0.47 | 1.85 | 1.48 | 2.50 | 1.93 | | |
| 476 | 204750.3 | 2021485 | 1.93 | 1.61 | 1.29 | 1.88 | 2.51 | 0.93 | | |
| 477 | 205232.1 | 1379153 | 1.01 | 2.35 | 1.01 | −0.32 | 1.99 | 1.51 | | |
| 478 | 212157.1 | 3130003 | 1.71 | −0.26 | 1.26 | 0.81 | 1.70 | 1.10 | | |
| 479 | 221846.4 | 4116336 | 1.98 | 0.37 | 1.53 | 1.32 | 2.01 | 1.68 | | |
| 480 | 222092.1 | 2154847 | 0.96 | 1.17 | 1.26 | 1.89 | 1.26 | 0.72 | | |
| 481 | 228350.1 | 2605849 | 2.35 | 2.29 | 1.65 | 2.17 | 2.51 | 1.04 | | |
| 482 | 229170.1 | 1818484 | 1.78 | 0.80 | 1.96 | 2.36 | 2.60 | 1.54 | | |
| 483 | 230820.1 | 3120378 | −1.46 | −1.67 | −0.81 | −1.03 | −1.48 | −2.03 | | |
| 484 | 231160.1 | 1848676 | 1.15 | −0.48 | 1.79 | 1.27 | 1.95 | 0.68 | | |
| 485 | 232212.1 | 1866738 | 1.60 | −0.26 | 1.92 | 1.26 | 1.15 | 0.37 | | |
| 486 | 234090.1 | 1718918 | 1.40 | 0.53 | 1.63 | 0.68 | 1.96 | 1.43 | | |
| 487 | 236432.1 | 3322032 | 2.00 | 0.72 | 0.20 | 1.77 | 2.98 | 1.20 | | |
| 488 | 236497.1 | 1682609 | −1.38 | 0.26 | −1.41 | −1.07 | 0.07 | −1.54 | | |
| 489 | 237208.4 | 993365 | −1.70 | −1.72 | −0.48 | −2.12 | −1.31 | −0.38 | | |
| 490 | 2484813CB1 | 1338090 | 2.88 | 2.67 | 2.25 | 3.21 | 3.10 | 0.85 | 1.17 | 0.0027 |
| 491 | 249104.1 | 2394947 | 2.11 | 1.29 | 2.09 | 1.53 | 2.06 | 2.20 | | |
| 492 | 287241.1 | 3412108 | 1.43 | 0.26 | 1.74 | 1.27 | 1.63 | 0.96 | | |
| 493 | 316571.1 | 5185101 | 2.02 | 1.59 | 1.56 | 1.48 | 0.98 | 2.43 | | |
| 494 | 331447.1 | 3363538 | −1.72 | −2.08 | −1.76 | −1.32 | 0.00 | −1.46 | | |
| 495 | 333127.1 | 153430 | −0.81 | −0.14 | −1.14 | −0.19 | −1.97 | −1.07 | | |
| 496 | 334025.5 | 3724351 | 2.01 | 1.60 | 0.68 | 1.80 | 1.29 | 0.63 | 1.20 | 0.242 |
| 497 | 334570.1 | 1296146 | 1.58 | 1.04 | 1.80 | 0.50 | 1.56 | 1.49 | | |
| 498 | 334814.1 | 1645745 | −1.72 | −1.96 | −0.87 | −1.58 | −1.70 | −0.72 | | |
| 499 | 335692.1 | 5023108 | −1.83 | 1.76 | −2.07 | −1.14 | −0.07 | −1.91 | | |
| 500 | 337407.1 | 3518117 | −1.29 | −1.53 | 0.00 | −2.12 | −1.20 | −0.63 | | |
| 501 | 337953.2 | 1378835 | 1.31 | 2.24 | 1.99 | 0.72 | 1.16 | 1.12 | | |
| 502 | 337953.5 | 1378835 | 1.31 | 2.24 | 1.99 | 0.72 | 1.16 | 1.12 | | |
| 503 | 346431.1 | 2723937 | 1.40 | 0.68 | 2.03 | 1.91 | 1.62 | 1.29 | | |
| 504 | 368357.1 | 5085077 | −2.46 | 0.65 | −1.58 | −1.16 | 0.00 | −2.56 | | |
| 505 | 390546.1 | 5339122 | 1.54 | 0.72 | 1.41 | 0.77 | 1.47 | 1.35 | | |
| 506 | 400267.1 | 4240272 | 1.63 | 1.17 | 0.43 | 2.19 | 2.19 | 0.72 | | |
| 507 | 402288.1 | 3871646 | −1.48 | 0.31 | −1.79 | −1.29 | 0.00 | −1.00 | | |
| 508 | 403407.2 | 1316042 | −1.03 | 1.56 | 1.35 | 0.92 | 1.98 | 0.96 | | |
| 509 | 405447.1 | 3220727 | −2.23 | −0.19 | 0.00 | −2.49 | 0.00 | −1.20 | | |
| 510 | 407485.1 | 5552541 | 1.03 | 1.07 | 2.48 | 2.08 | 1.74 | 0.85 | | |
| 511 | 427832.19 | 3692235 | −2.67 | 2.89 | −3.48 | −1.68 | 0.00 | −3.34 | | |
| 512 | 477913.1 | 2593092 | 0.85 | 1.35 | 1.03 | 1.10 | 1.98 | 1.79 | | |
| 513 | 902956.2 | 2607572 | −1.98 | 0.85 | −2.17 | −1.20 | −0.14 | −2.23 | | |
| 514 | 979367.4 | 2998372 | 0.85 | −0.38 | 1.54 | 1.50 | 1.81 | 1.07 | | |
| 515 | 979367.5 | 2998372 | 0.85 | −0.38 | 1.54 | 1.50 | 1.81 | 1.07 | | |
| 516 | 982861.1 | 3888854 | −2.26 | 2.40 | −3.00 | −1.07 | 0.48 | −2.70 | | |
| 517 | 983351.1 | 1819244 | 2.43 | 0.37 | 3.20 | 2.06 | 2.71 | 1.96 | | |
| 518 | 984637.1 | 1610772 | −2.52 | 1.77 | −2.94 | −1.65 | 0.26 | −3.06 | | |
| 519 | 984900.1 | 1802185 | −1.26 | −1.51 | −0.53 | −1.85 | −2.00 | −1.22 | | |

TABLE 2

| SEQ | | | | | |
|---|---|---|---|---|---|
| ID NO | Template ID | Clone ID | Genbank ID | E−value | Homolog Description |
| 1 | 1867417CB1 | 1357231 | g2668414 | 0 | topoisomersae II [Sus scrofa] |
| 2 | 1970111CB1 | 1970111 | g286013 | 0 | KIAA0008 [Homo sapiens] |
| 3 | 959142CB1 | 1930447 | g2995138 | 0 | thrombospondin 2 [Bos taurus] |
| 4 | 064684.7 | 1824717 | g3413861 | 0 | Homo sapiens mRNA for KIAA0450 protein, complete cds. |
| 5 | 245093.34 | 958486 | g393319 | 0 | osteoblast specific factor 2 [Homo sapiens] |
| 6 | 331908.5 | 1995457 | g63114 | 0 | B-cadherin [Gallus gallus] |
| 7 | 1329880.35 | 5150602 | g30058 | 1.00E−142 | prepro-alpha-1 type 3 collagen [Homo sapiens] |
| 8 | 963555.1 | 2820294 | g30058 | 1.00E−140 | prepro-alpha-1 type 3 collagen [Homo sapiens] |
| 9 | 199471.2 | 2047549 | g1575534 | 1.00E−112 | Mad2 [Homo sapiens] |
| 10 | 048849.1 | 1358605 | g5669134 | 6.00E−37 | Homo sapiens constitutive fragile region FRA3B sequence. |
| 11 | 278283.1 | 4180161 | g29584 | 3.00E−12 | Human mRNA for alpha 1 (III) collagen fragment (aa 892-1023). |
| 12 | 978433CB1 | 1869068 | g2088834 | 1.00E−05 | F59E12.12 gene product [Caenorhabditis elegans] |
| 13 | 611514CB1 | 605219 | g1374792 | 0 | selenium-binding protein [Homo sapiens] |
| 14 | 1382907.35 | 1888708 | g165490 | 0 | myosin heavy chain [Oryctolagus cuniculus] |
| 15 | 350509.2 | 1510413 | g1699038 | 0 | ABC3 [Homo sapiens] |
| 16 | 2512879CB1 | 2512879 | g178092 | 0 | alcohol dehydrogenase 1 [Homo sapiens] |
| 17 | 241123.1 | 3876732 | g1834493 | 0 | flavin-containing monooxygenase 2 [Homo sapiens] |
| 18 | 247817.4 | 1480063 | g186675 | 0 | receptor tyrosine kinase [Homo sapiens] |
| 19 | 1674368CB1 | 1241484 | g188676 | 0 | mannose receptor precursor [Homo sapiens] |
| 20 | 343963.1 | 2671006 | g1907329 | 0 | angiopoietin-1 [Mus musculus] |
| 21 | 273154CB1 | 2899786 | g202592 | 0 | prealpha-2-macroglobulin [Rattus norvegicus] |
| 22 | 331508.4 | 1967759 | g2222794 | 0 | VE−Cadherin [Sus scrofa] |
| 23 | 1137924.1 | 1480479 | g2337883 | 0 | pyruvate dehydrogenase kinase isoform 4 [Homo sapiens] |
| 24 | 247168.4 | 2278925 | g2338748 | 0 | oxidoreductase [Homo sapiens] |
| 25 | 2275817CB1 | 224996 | g2853224 | 0 | skeletal muscle LIM-protein FHL1 [Homo sapiens] |
| 26 | 2717806CB1 | 4918603 | g29721 | 0 | catalase (aa 1-527) [Homo sapiens] |
| 27 | 407624.1 | 3771805 | g3360425 | 0 | Homo sapiens clone 23822 mRNA sequence. |
| 28 | 3950154CB1 | 2748163 | g34383 | 0 | precursor protein [Homo sapiens] |
| 29 | 3741842CB1 | 1988080 | g3676522 | 0 | prostaglandin transporter [Homo sapiens] |
| 30 | 221055.4 | 2718391 | g4092861 | 0 | p53 regulated PA26-T3 nuclear protein [Homo sapiens] |
| 31 | 379425CB1 | 433622 | g409977 | 0 | nontransmembrane protein tyrosine phosphatase, ERP [mice, liver, Peptide, |
| | | | | | 367 aa][Mus sp.] |
| 32 | 1685090CB1 | 2721792 | g4585372 | 0 | Wnt inhibitory factor-1 [Mus musculus] |
| 33 | 350476.1 | 123312 | g4588918 | 0 | serotonin transporter [Bos taurus] |
| 33 | 350476.1 | 4044520 | g4588918 | 0 | serotonin transporter [Bos taurus] |
| 34 | 404278.1 | 3116479 | g4884242 | 0 | Homo sapiens mRNA; cDNA DKFZp564G112 (from clone DKFZp564G112). |
| 35 | 1250492CB1 | 1361644 | g547484 | 0 | sodium-dependent phosphate transporter [Bos taurus] |
| 36 | 200095.2 | 3125685 | g547484 | 0 | sodium-dependent phosphate transporter [Bos taurus] |
| 37 | 997405.3 | 1240444 | g6006498 | 0 | dTDP-4-keto-6-deoxy-D-glucose 4-reductase [Homo sapiens] |
| 38 | 235369.10 | 1965041 | g6468391 | 0 | dJ365O12.1 (KIAA0758 protein) [Homo sapiens] |
| 39 | 236587.4 | 1362125 | g6572260 | 0 | bK65A6.2 (novel Sushi domain (SCR repeat) containing protein |
| | | | | | similar to Mucins) (Homo sapiens] |
| 40 | 1135936.1 | 4271973 | g6599288 | 0 | Homo sapiens mRNA; cDNA DKFZp586A0421 |
| | | | | | (from clone DKFZp586A0421). |
| 41 | 251123.6 | 3557441 | g6636099 | 0 | NADH/NADPH thyroid oxidase p138-tox [Homo sapiens] |
| 41 | 251123.6 | 2600963 | g6636099 | 0 | NADH/NADPH thyroid oxidase p138-tox [Homo sapiens] |
| 42 | 2547002CB1 | 2475740 | g7363342 | 0 | chemokine receptor [Homo sapiens] |
| 43 | 251123.8 | 3557441 | g8163932 | 0 | NADPH thyroid oxidase 2 [Canis familiaris] |
| 44 | 1000172.35 | 3645309 | g338048 | 1.00E−180 | Human pulmonary surfactant-associated protein |
| | | | | | SP-A (SFTP1) gene, complete cds. |
| 45 | 2921920CB1 | 1498363 | g7019846 | 1.00E−159 | unnamed protein product [Homo sapiens] |
| 46 | 631645CB1 | 1274935 | g1655592 | 1.00E−152 | folate receptor [Homo sapiens] |
| 46 | 631645CB1 | 1577756 | g1655592 | 1.00E−152 | folate receptor [Homo sapiens] |
| 47 | 1507546CB1 | 3507734 | g292507 | 1.00E−144 | surfactant protein D [Homo sapiens] |
| 48 | 1508437CB1 | 1376121 | g1483627 | 1.00E−140 | folate receptor [Homo sapiens] |
| 48 | 1508437CB1 | 1607091 | g1483627 | 1.00E−140 | folate receptor [Homo sapiens] |
| 49 | 1100669.4 | 1675122 | g825722 | 1.00E−116 | tetranectin [Homo sapiens] |
| 50 | 124600CB1 | 1578941 | g1203984 | 1.00E−108 | NAD +− dependent 15-hydroxyprostaglandin dehydrogenase [Homo sapiens] |
| 51 | 470771.11 | 322066 | g190856 | 1.00E−100 | GTPase activating protein [Homo sapiens] |
| 52 | 273259.4 | 2201411 | g7768703 | 5.00E−93 | Homo sapiens genomic DNA, chromosome 21q, section 38/105. |
| 53 | 5170638CB1 | 4014022 | g339946 | 2.00E−81 | slow twitch skeletal/cardiac muscle troponin C [Homo sapiens] |
| 54 | 1330170.3 | 4221057 | g4038450 | 1.00E−77 | alpha one globin [Homo sapiens] |
| 55 | 1383898.2 | 1499549 | g184834 | 1.00E−76 | insulin-like growth factor [Homo sapiens] |
| 56 | 019238.3 | 1430507 | g6841220 | 3.00E−73 | HSPC285 [Homo sapiens] |
| 57 | 1102296.5 | 3026658 | g4038450 | 5.00E−73 | alpha one globin [Homo sapiens] |
| 58 | 995673.3 | 2149968 | g403S450 | 9.00E−71 | alpha one globin [Homo sapiens] |
| 58 | 995673.3 | 4215545 | g4038450 | 9.00E−71 | alpha one globin [Homo sapiens] |
| 59 | 312256CB1 | 313697 | g4519602 | 3.00E−59 | IGSF4 [Homo sapiens] |
| 60 | 199183.2 | 2763310 | g6652812 | 2.00E−55 | putative secreted protein XAG [Homo sapiens] |
| 61 | 289671.40 | 3678546 | g7230514 | 2.00E−55 | extracellular glutathione peroxidase [Homo sapiens] |
| 62 | 154403.1 | 2050104 | g530140 | 7.00E−50 | Homo sapiens iduronate sulphate sulphatase (IDS) gene, |
| | | | | | complete cds. |
| 63 | 086518.22 | 1987759 | g6137108 | 5.00E−47 | RGC32 Homo sapiens] |
| 64 | 1383156.10 | 311197 | g262770 | 2.00E−43 | type II surfactant protein C, type II SP-C [rabbits, lung, Peptide, |
| | | | | | 189 aa] [Oryctolagus cuniculus] |
| 65 | 1100669.3 | 1675122 | g825722 | 4.00E−41 | tetranectin [Homo sapiens] |
| 66 | 1330148.1 | 3867795 | g386765 | 7.00E−36 | hba1 alpha globin [Homo sapiens] |
| 67 | 1436702CB1 | 1358185 | g180501 | 5.00E−30 | channel-like integral membrane protein [Homo sapiens] |
| 68 | 3141226CB1 | 3625857 | g183629 | 2.00E−29 | cytokine gro-beta [Homo sapiens] |
| 69 | 289671.44 | 970905 | g2654264 | 8.00E−29 | glutathione peroxidase [Homo sapiens] |
| 70 | 197927.7 | 2417149 | g8176600 | 1.00E−16 | Homo sapiens TCL6 gene, exon 11. |
| 71 | 1134834.2 | 1911306 | g5714696 | 7.00E−14 | alpha 2 delta calcium channel subunit [Mus musculus] |
| 72 | 200273.1 | 2721144 | g7229101 | 1.00E−10 | down-regulated in gastric cancer [Homo sapiens] |
| 73 | 477974.1 | 3117677 | g37519 | 9.00E−09 | H. sapiens U13 snRNA pseudogene U13.12A. |
| 74 | 235095.7 | 1809377 | g7529597 | 2.00E−07 | dJ402N21.2 (novel protein with MAM domain) [Homo sapiens] |
| 75 | 407593.1 | 205053 | | | Incyte Unique |
| 76 | 332301.1 | 1417150 | | | Incyte Unique |
| 77 | 407699.4 | 1919860 | | | Incyte Unique |
| 78 | 405501.1 | 2593077 | | | Incyte Unique |
| 79 | 482541.2 | 2685454 | | | Incyte Unique |
| 80 | 237026.3 | 2963374 | | | Incyte Unique |
| 81 | 443605.4 | 3556587 | | | Incyte Unique |
| 82 | 443605.1 | 53556587 | | | Incyte Unique |
| 83 | 029997.1 | 3638532 | | | Incyte Unique |
| 84 | 331743.9 | 789903 | g1136394 | 0 | There are three putative hydrophobic domains in the central region. |
| | | | | | [Homo sapiens] |
| 85 | 337334.1 | 3220029 | g155084 | 0 | kinesin-related protein [Homo sapiens] |
| 86 | 410438.2 | 1879818 | g1200247 | 0 | perforin [Mus musculus] |
| 87 | 453004.3 | 22394888 | g1246779 | 0 | calpain [Mus musculus] |
| 88 | 236319.2 | 3044230 | g1399032 | 0 | copper monamine oxidase [Homo sapiens] |
| 89 | 978118.5 | 2508618 | g1439565 | 0 | chitinase [Homo sapiens] |
| 90 | 1383177.16 | 1646505 | g1469874 | 0 | The KIAA0146 gene product is novel. [Homo sapiens] |
| 91 | 1383298.1 | 1510539 | g1490314 | 0 | peroxisome proliferator activated receptor gamma [Homo sapiens] |
| 92 | 1000222.20 | 1729693 | g1498255 | 0 | replication factor C, 37-kDa subunit [Homo sapiens] |
| 93 | 1096863.27 | 1303863 | g1519394 | 0 | leptin receptor [Homo sapiens] |
| 94 | 245334.1 | 2380412 | g1688258 | 0 | collagenase [Homo sapiens] |
| 95 | 1656674CB1 | 2222802 | g1698708 | 0 | mast cell carboxypeptidase A precursor [Rattus norvegicus] |
| 96 | 522678CB1 | 2199851 | g177205 | 0 | 92 kDa type IV collagenase [Homo sapiens] |
| 97 | 1424985CB1 | 2150615 | g1778069 | 0 | zyginI [Rattus norvegicus] |
| 98 | 1468237CB1 | 1573505 | g178277 | 0 | S-adenosylhomocysteine hydrolase [Homo sapiens] |
| 99 | 337008.1 | 1599272 | g180494 | 0 | butyrylcholinesterase (EC 3.1.1.8) [Homo sapiens] |
| 100 | 444850.9 | 2860704 | g183002 | 0 | guanylate binding protein isoform I [Homo sapiens] |
| 101 | 786284CB1 | 1458210 | g183138 | 0 | gamma-glutamyl transpeptidase (EC 2.3.2.2) [Homo sapiens] |
| 102 | 1352170CB1 | 11988092 | g187240 | 0 | leukocyte surface protein [Homo sapiens] |
| 103 | 1815320CB1 | 1742116 | g1899259 | 0 | CX3C chemokine precursor [Homo sapiens] |
| 104 | 331192.11 | 2007554 | g1903384 | 0 | preferentially expressed antigen of melanoma |
| | | | | | [Homo sapiens] |
| 105 | 344053.5 | 2928545 | g191992 | 0 | APC [Mus musculus] |
| 106 | 411188.2 | 4247796 | g202874 | 0 | AMPA selective glutamate receptor [Rattus norvegicus] |
| 107 | 331510.4 | 3814138 | g2071974 | 0 | phospholipase D1 [Cricetulus griseus] |
| 108 | 492750CB1 | 2732630 | g2125814 | 0 | serine/threonine protein kinase [Homo sapiens] |
| 109 | 348912.4 | 1716655 | g219936 | 0 | NCA-W272 [Homo sapiens] |
| 110 | 2680109CB1 | 2125020 | g255098 | 0 | transmembrane secretory component, SC, polyIg receptor [Homo sapiens] |
| 110 | 2680109CB1 | 2242677 | g255098 | 0 | transmembrane secretory component, SC, poly-Ig receptor [Homo sapiens] |
| 111 | 512261CB1 | 512261g | 2570531 | 0 | beta2-adrenergic receptor [Homo sapiens] |
| 112 | 232719.2 | 1848259 | g2582830 | 0 | alpha1 integrin [Gallus gallus] |
| 113 | 369664.2 | 3119171 | g2642133 | 0 | neuronal apoptosis inhibitory protein [Homo sapiens] |
| 114 | 021042.1 | 2879922 | g2735714 | 0 | Homo sapiens pro-alpha 2(I) collagen (COL1A2) gene, complete cds. |
| 115 | 418805.7 | 1637320 | g2735857 | 0 | cAMP-specific phosphodiesterase PDE4D5 [Homo sapiens] |
| 116 | 1094000.4 | 1397816 | g285926 | 0 | human endothelin-B receptor [Homo sapiens] |
| 117 | 1094000.5 | 1380927 | g285926 | 0 | human endothelin-B receptor [Homo sapiens] |
| 118 | 1222734CB1 | 1300701 | g285926 | 0 | human endothelin-B receptor [Homo sapiens] |
| 118 | 1222734CB1 | 1397816 | g285926 | 0 | human endothelin-B receptor [Homo sapiens] |
| 119 | 411205.5 | 1420883 | g2865521 | 0 | protein regulating cytokinesis 1; PRC1 [Homo sapiens] |
| 120 | 1092777.6 | 1643711 | g292823 | 0 | Homo sapiens receptor protein-tyrosine kinase (TEK) mRNA, complete cds. |
| 121 | 234358.5 | 1996180 | g293332 | 0 | ect2 [Mus musculus] |
| 122 | 1092777.7 | 1643711 | g296578 | 0 | receptor tyrosine kinase [Bos taurus] |
| 123 | 4180444CB1 | 3872317 | g3025335 | 0 | sucrase-isomaltase [Suncus murinus] |
| 124 | 1987983CB1 | 690819 | g3061284 | 0 | p67phox [Mus musculus] |
| 125 | 1398420.2 | 4655050 | g306799 | 0 | pregnancy-specific beta-glycoprotein e [Homo sapiens] |
| 126 | 1749102CB1 | 1749102 | g306956 | 0 | indoleamine 2,3-dioxygenase (IDO) (EC 1.13.11.17) |
| | | | | | [Homo sapiens] |
| 127 | 2965804CB1 | 2965804 | g309266 | 0 | glycerophosphate dehydrogenase [Mus musculus] |
| 128 | 210095.21 | 1285926 | g3172150 | 0 | BGPc_HUMAN [Homo sapiens] |
| 129 | 1555752CB1 | 42248 | g3192879 | 0 | MAD3-like protein kinase [Homo sapiens] |
| 130 | 110245.1 | 3028719 | g3213194 | 0 | Homo sapiens serine-threonine kinase (BTAK) gene, partial cds. |
| 131 | 009476CB1 | 2017386 | g3243035 | 0 | RuvB-like protein RUVBLI [Homo sapiens] |
| 132 | 1560874CB1 | 3815942 | g3283045 | 0 | CDC45L [Homo sapiens] |
| 133 | 3571894CB1 | 2132715 | g3288916 | 0 | aortic carboxypeptidase-like protein ACLP [Homo sapiens] |
| 134 | 349622.1 | 2055903 | g338422 | 0 | Human small proline rich protein (sprII) mRNA, clone 930. |
| 135 | 002940CB1 | 1679482 | g3402293 | 0 | aurora and IPL1-like midbody-associated protein kinase-1 [Homo sapiens] |
| 136 | 1362466CB1 | 1834502 | g34754 | 0 | put. ribosomal protein L3 (AA 1 - 348) [Homo sapiens] |
| 137 | 383376.19 | 4461157 | g37947 | 0 | VWF pre-pro-polypeptide (−22 to 2791) [Homo sapiens] |
| 138 | 1382961.3 | 3184882 | g386848 | 0 | keratin [Homo sapiens] |
| 139 | 1382961.15 | 4382348 | g3868804 | 0 | cytokeratin 17 [Mus musculus] |
| 140 | 1454852CB1 | 793403 | g38688040 | 0 | cytokeratin 17 [Mus musculus] |
| 141 | 2161632CB1 | 954057 | g39167330 | 0 | lysyl oxidase homolog [Perca flavescens] |
| 142 | 2058013CB1 | 1968413 | g401763 | 0 | ataxia-telangiectasia group D-associated protein [Homo sapiens] |
| 143 | 235333.1 | 1684632 | g402197 | 0 | ALK-1 [Homo sapiens] |
| 144 | 702628CB1 | 2418629 | g4049492 | 0 | Cdc6-related protein [Mus musculus] |
| 145 | 1383415.3 | 1420380 | g4098297 | 0 | Koc1 [Homo sapiens] |
| 146 | 236309.1 | 2925376 | g4102034 | 0 | Homo sapiens LNCaP mRNA, putative 3′UTR sequence. |
| 147 | 344868.13 | 3333118 | g4105617 | 0 | cytosolic NADP-dependent isocitrate dehydrogenase [Microtus ochrogaster] |
| 148 | 368869.1 | 5100602 | g4138921 | 0 | Homo sapiens promyclocytic leukemia zinc |
| | | | | | finger protein (PLZF) gene, complete cds. |
| 149 | 3256566CB1 | 1666069 | g4204915 | 0 | ladinin [Homo sapiens] |
| 150 | 4104673CB1 | 4104673 | g4261713 | 0 | chlordecone reductase homolog [Homo sapiens] |
| 151 | 345511.2 | 4243318 | g433491 | 0 | H. sapiens HLA-E gene. |
| 152 | 1723834CB1 | 1723834 | g4347S3 | 0 | KIAA0030 [Homo sapiens] |
| 153 | 078756CB1 | 490795 | g435776 | 0 | retinoblastoma binding protein 1, RBP1 [Homo sapiens] |
| 154 | 331749.3 | 1628341 | g453368 | 0 | Human maspin mRNA, complete cds. |
| 155 | 350528.7 | 1429293 | g456353 | 0 | intestinal VIP receptor related protein [[Homo sapiens] |
| 156 | 1099159.1 | 1519404 | g4755084 | 0 | Homo sapiens pro alpha 1(I) collagen (COL1A1) gene, |
| | | | | | complete cds. |
| 157 | 1530186CB1 | 3715059 | g4826465 | 0 | dJ287G14.2 (PUTATIVE novel seven transmembrane domain |
| | | | | | protein) [Homo sapiens] |
| 158 | 1092387.11 | 2674527 | g4871330 | 0 | Human hepatic dihydrodiol dehydrogenase gene, exon 9. |
| 159 | 107939.2 | 2313581 | g4902678 | 0 | hypothetical protein [Homo sapiens] |
| 160 | 899248.6 | 1832584 | g4929268 | 0 | LOMP protein [Homo sapiens] |
| 161 | 2959521CB1 | 87727 | g505102 | 0 | KIAA0062 [Homo sapiens] |
| 162 | 3331519CB1 | 2197965 | g512414 | 0 | tie receptor tyrosine kinase [Homo sapiens] |
| 163 | 1453334CB1 | 1445547 | g512447 | 0 | uPA [Homo sapiens] |
| 164 | 2798854CB1 | 4385292 | g5262584 | 0 | hypothetical protein [Homo sapiens] |
| 165 | 227961.1 | 2742979 | g5262S97 | 0 | hypothetical protein [Homo sapiens] |
| 166 | 1427470CB1 | 1430862 | g537514 | 0 | arylacetaniide deacetylase [Homo sapiens] |
| 167 | 206250.6 | 2180220 | g5457171 | 0 | dA159A1.1 (novel protein) [Homo sapiens] |
| 168 | 995068.15 | 893230 | g546085 | 0 | cytoplasmic antiproteinase, CAP = 38 kda intracellular |
| | | | | | serine proteinase inhibitor [Homo sapiens] |
| 169 | 2545475CB1 | 1981569 | g561722 | 0 | monocarboxylate transporter 1 [Homo sapiens] |
| 170 | 1502559CB1 | 1502559 | g5678815 | 0 | inositol polyphosphate 1-phosphatase [Homo sapiens] |
| 171 | 2239738CB1 | 2239738 | g5809682 | 0 | carboxypeptidase M precursor [Homo sapiens] |
| 172 | 347572.1 | 1501621 | g5817160 | 0 | hypothetical protein [Homo sapiens] |
| 173 | 977985.10 | 1445387 | g5912203 | 0 | Homo sapiens mRNA; cDNA DKFZp564N1164 (from |
| | | | | | clone DKFZp564N1164). |
| 174 | 236582.2 | 2720359 | g5926698 | 0 | Homo sapiens genomic DNA, chromosome 6p21.3, HLA Class I region, |
| | | | | | section 10/20. |
| 175 | 403676.1 | 1656490 | g5926709 | 0 | Homo sapiens genomic DNA, chromosome 6p21.3, HLA Class I region, |
| | | | | | section 19/20. |
| 176 | 138472CB1 | 2373085 | g598143 | 0 | alcohol dehydrogenase beta-3 subunit [Homo sapiens] |
| 177 | 253672.11 | 2851539 | g6016837 | 0 | Homo sapiens mRNA for 14-3-3gamma, complete cds. |
| 178 | 201928.3 | 2126712 | g6172223 | 0 | alias: DIL-2; chromosome 20 open reading frame 2 [Homo sapiens] |
| 179 | 198643.1 | 2187262 | g6273492 | 0 | Homo sapiens Spast gene for spastin protein. |
| 180 | 979448.2 | 1497012 | g6331294 | 0 | KIAA 1275 protein [Homo sapiens] |
| 181 | 460745.7 | 1381145 | g6331308 | 0 | KIAA 1277 protein [Homo sapiens] |
| 182 | 399300.18 | 2842978 | g6403463 | 0 | alpha-catulin [Homo sapiens] |
| 183 | 248091.1 | 1607083 | g6467177 | 0 | novel member of chitinase family [Homo sapiens] |
| 184 | 4030737CB1 | 2440848 | g6518913 | 0 | Bit [Homo sapiens] |
| 185 | 3012575CB1 | 3012575 | g6531404 | 0 | mutant desmin [Homo sapiens] |
| 186 | 221827.1 | 2057406 | g6599073 | 0 | caveolin-1/-2 locus, Contig1, D7S522 [Homo sapiens] |
| 187 | 238660.5 | 1604437 | g6624920 | 0 | DMBT1 prototype [Homo sapiens] |
| 188 | 2171401CB1 | 2055814 | g6642925 | 0 | ERO1L [Mus musculus] |
| 189 | 1420940CB1 | 1603408 | g6642929 | 0 | 3′phosphoadenosine 5′-phosphosulfate synthase 2b isoform [Homo sapiens] |
| 190 | 1383160.14 | 1834906 | g6807690 | 0 | hypothetical protein [Homo sapiens] |
| 191 | 233331.8 | 2154725 | g6808077 | 0 | hypothetical protein [Homo sapiens] |
| 192 | 338217.10 | 1989545 | g6808502 | 0 | C11orf9 [Homo sapiens] |
| 193 | 5982278CB1 | 2640427 | g683536 | 0 | CHO1 antigen [Cricetulus griseus] |
| 194 | 1097030.1 | 1760232 | g688384 | 0 | Differentiation-stimulating factor receptor, |
| | | | | | leukemia inhibitory factor receptor, LIF receptor [Mus sp.] |
| 195 | 1100509.4 | 2215183 | g6906729 | 0 | Cas and HEF1 associated signal transducer [Mus musculus] |
| 196 | 199671.1 | 1694119 | g7018527 | 0 | Homo sapiens mRNA; cDNA DKFZp762M127 (from clone DKFZp762M127). |
| 197 | 232137.20 | 2053221 | g7020309 | 0 | unnamed protein product [Homo sapiens] |
| 198 | 898608.2 | 3112520 | g7020365 | 0 | unnamed protein product [Homo sapiens] |
| 199 | 411373.3 | 1625856 | g7243117 | 0 | KIAA1368 protein [Homo sapiens] |
| 200 | 1092387.17 | 2070554 | g7328944 | 0 | 20 alpha-hydroxysteroid dehydrogenase [Homo sapiens] |
| 201 | 201906.5 | 3733666 | g7416858 | 0 | MBIP [Homo sapiens] |
| 202 | 3607580CB1 | 3607580 | g747970 | 0 | angiotensin II type 2 receptor [Homo sapiens] |
| 203 | 991163CB1 | 1809178 | g7578919 | 0 | plasmic transmembrane protein X [Mus musculus] |
| 204 | 1309633.1 | 1880421 | g7717462 | 0 | AgX-1 antigen [Homo sapiens] |
| 205 | 1384719.29 | 4372330 | g780261 | 0 | lactate dehydrogenase-A [Homo sapiens] |
| 206 | 178250.2 | 3075015 | g8216989 | 0 | putative cell cycle control protein [Homo sapiens] |
| 207 | 1099945.13 | 1911742 | g833853 | 0 | versican V2 core protein precursor [Homo sapiens] |
| 208 | 2172334CB1 | 2172334 | g840817 | 0 | gp130 [Mus musculus] |
| 209 | 253946.17 | 3625189 | g840817 | 0 | gp130 [Mus musculus] |
| 210 | 008513.49 | 2057510 | g908790 | 0 | keratin type II [Homo sapiens] |
| 211 | 1092257.2 | 1447795 | g912488 | 0 | gut-enriched Kruppel-like factor [Mus musculus] |
| 211 | 1092257.2 | 1962235 | g912488 | 0 | gut-enriched Kruppel-like factor [Mus musculus] |
| 211 | 1092257.2 | 3440567 | g912488 | 0 | gut-enriched Kruppel-like factor [Mus musculus] |
| 212 | 4349106CB1 | 2820861 | g912488 | 0 | gut-enriched Kruppel-like factor [Mus musculus] |
| 213 | 1814803CB1 | 1698951 | g1296503 | 1.00E−179 | uracil-DNA-glycosylase, UNG1 [Homo sapiens] |
| 214 | 1141764.8 | 1926114 | g7243185 | 1.00E−179 | KIAA1402 protein [Homo sapiens] |
| 215 | 406531.1 | 1603580 | g339560 | 1.00E−178 | bone morphogenetic protein 5 [Homo sapiens] |
| 216 | 429307.4 | 3144021 | g4321794 | 1.00E−177 | Homo sapiens keratin 16 (KRT16A) gene, complete cds. |
| 217 | 239515.9 | 1675369 | g4151205 | 1.00E−176 | neurogenic extracellular slit protein Slit2 [Homo sapiens] |
| 218 | 1089210.1 | 5033671 | g4261710 | 1.00E−176 | chlordecone reductase [Homo sapiens] |
| 219 | 010796.18 | 1445895 | g2467300 | 1.00E−173 | phosphatidic acid phosphatase 2b [Homo sapiens] |
| 220 | 3752346CB1 | 1378037 | g3334761 | 1.00E−173 | ribonuclease HI large subunit [Homo sapiens] |
| 221 | 2729828CB1 | 1858171 | g34033 | 1.00E−173 | keratin 13 [Homo sapiens] |
| 222 | 1648579CB1 | 3142624 | g187291 | 1.00E−172 | MAD3 [Homo sapiens] |
| 223 | 1454418CB1 | 1468660 | g29839 | 1.00E−172 | CDC2 polypeptide (CDC2) (AA 1-297) [Homo sapiens] |
| 224 | 995529.10 | 2957476 | g29839 | 1.00E−172 | CDC2 polypeptide (CDC2) (AA 1-297) [Homo sapiens] |
| 225 | 2126751CB1 | 2126751 | g1923256 | 1.00E−167 | 26S proteasome-associated pad1 homolog [Homo sapiens] |
| 226 | 569648CB1 | 2720467 | g5231092 | 1.00E−163 | macrophage receptor [Homo sapiens] |
| 227 | 1352789CB1 | 3000146 | g7023570 | 1.00E−163 | unnamed protein product [Homo sapiens] |
| 228 | 1635966CB1 | 2046165 | g6318544 | 1.00E−162 | retinal short-chain dehydrogenase/reductase retSDR2 [Homo sapiens] |
| 229 | 349415.6 | 3074415 | g1125016 | 1.00E−159 | MAGE−6 protein [Homo sapiens] |
| 230 | 406438.14 | 2797787 | g1552328 | 1.00E−158 | TFG [Homo sapiens] |
| 231 | 1471808CB1 | 3074415 | g533523 | 1.00E−158 | MAGE−6 antigen [Homo sapiens] |
| 232 | 245595.1 | 2052480 | g7328092 | 1.00E−158 | hypothetical protein [Homo sapiens] |
| 233 | 2784232CB1 | 3151158 | g7023868 | 1.00E−157 | unnamed protein product [Homo sapiens] |
| 234 | 2742913CB1 | 2516950 | g179795 | 1.00E−156 | carbonic anhydrase II [Homo sapiens] |
| 235 | 1509972CB1 | 4047785 | g338327 | 1.00E−156 | pulmonary surfactant-associated protein SP-B [Homo sapiens] |
| 236 | 1097638.1 | 1903760 | g187094 | 1.00E−155 | Human low density lipoprotein receptor gene, exon 18. |
| 237 | 900035.58 | 1900306 | g1418928 | 1.00E−154 | prepro-alpha1(I) collagen [Homo sapiens] |
| 237 | 900035.58 | 5186773 | g1418928 | 1.00E−154 | prepro-alpha1(I) collagen [Homo sapiens] |
| 238 | 1075592.6 | 1423848 | g505589 | 1.00E−154 | [Human insulin-like growth factor binding protein 5 |
| | | | | | (IGFBP5) gene], gene product [Homo sapiens] |
| 239 | 984540.1 | 1393337 | g7657926 | 1.00E−154 | UDP-N-acetyl-alpha-D-galactosamine: polypeptide |
| | | | | | N-acetylgalactosaminyltransferase 8 [Homo sapiens] |
| 240 | 378633.40 | 1821938 | g1182067 | 1.00E−153 | tryptase precursor [Homo sapiens] |
| 241 | 477387.7 | 3100048 | g469478 | 1.00E−152 | SM-20 [Rattus norvegicus] |
| 242 | 347049.9 | 2057158 | g1161562 | 1.00E−151 | stomatin [Homo sapiens] |
| 243 | 1097717.18 | 5065213 | g1732423 | 1.00E−151 | C9 [Homo sapiens] |
| 244 | 2832214CB1 | 2832214 | g1732423 | 1.00E−151 | C9 [Homo sapiens] |
| 245 | 2947513CB1 | 5984038 | g1732423 | 1.00E−151 | C9 [Homo sapiens] |
| 246 | 1655369CB1 | 2056840 | g3319988 | 1.00E−151 | TOM1 [Mus musculus] |
| 247 | 039170.3 | 2418484 | g1029482 | 1.00E−150 | H. sapiens CpG island DNA genomic Mse1 fragment, |
| | | | | | clone 33e12, reverse read cpg33e12rt1f. |
| 248 | 1074926.1 | 2947513 | g176959 | 1.00E−150 | P. troglodytes triose-phosphate isomerase (TPI) gene, complete cds. |
| 249 | 344297.5 | 62144 | g413911 | 1.00E−149 | Rat cyclin E [Rattus rattus] |
| 250 | 410721.1 | 4688 | g416115 | 1.00E−149 | MAGE−1 [Homo sapiens] |
| 251 | 1121097.1 | 1450641 | g7023561 | 1.00E−147 | unnamed protein product [Homo sapiens] |
| 252 | 1097450.22 | 3374194 | g307042 | 1.00E−145 | gamma-interferon-inducible protein precursor [Homo sapiens] |
| 253 | 238533.1 | 2790947 | g7673566 | 1.00E−144 | CLIC5 [Homo sapiens] |
| 254 | 1097080.8 | 2694381 | g2674195 | 1.00E−143 | polymerase I-transcript release factor; PTRF [Mus musculus] |
| 255 | 1097450.20 | 3374194 | g307042 | 1.00E−142 | gamma-interferon-inducible protein precursor [Homo sapiens] |
| 256 | 2514988CB1 | 1886886 | g178853 | 1.00E−141 | apolipoprotein E [Homo sapiens] |
| 257 | 3130485CB1 | 1987238 | g3885376 | 1.00E−140 | Homo sapiens mRNA expressed only in placental villi, clone SMAP31. |
| 258 | 1330234.11 | 5392723 | g189151 | 1.00E−138 | nephropontin [Homo sapiens] |
| 259 | 1943624CB1 | 1943624 | g439603 | 1.00E−137 | Rad [Homo sapiens] |
| 260 | 2189816CB1 | 2189816 | g478887 | 1.00E−137 | folate receptor 3 (gamma) [Homo sapiens] |
| 261 | 039027.1 | 3861522 | g5689824 | 1.00E−137 | Homo sapiens mnRNA full length insert cDNA clone EUROIMAGE 295344 |
| 262 | 2502336CB1 | 2507719 | g533528 | 1.00E−136 | MAGE−9 antigen [Homo sapiens] |
| 263 | 3592543CB1 | 5066393 | g386839 | 1.00E−135 | JUN [Homo sapiens] |
| 264 | 399589.1 | 2070856 | g7688215 | 1.00E−135 | dJ788L20.1 (hepatocyte nuclear factor 3, beta) [Homo sapiens] |
| 265 | 2175401CB1 | 1833174 | g4262298 | 1.00E−132 | integral membrane protein 2A [Mus musculus] |
| 266 | 084455.9 | 1356268 | g3329482 | 1.00E−131 | Sox-like transcriptional factor [Homo sapiens] |
| 267 | 233041.5 | 2060622 | g36652 | 1.00E−128 | syndecan [Homo sapiens] |
| 268 | 1382961.5 | 3184882 | g386848 | 1.00E−128 | keratin [Homo sapiens] |
| 269 | 1518310CB1 | 1518310 | g4481753 | 1.00E−127 | connexin 26 [Homosapiens] |
| 270 | 1517817CB1 | 1402078 | g4894854 | 1.00E−127 | complement C1q A chain precursor [Homo sapiens] |
| 271 | 3090387CB1 | 1714684 | g7325554 | 1.00E−127 | tumor necrosis factor-related death ligand-1alpha [Homo sapien] |
| 272 | 3836893CB1 | 3836893 | g8050527 | 1.00E−125 | triggering receptor expressed on monocytes 1 [Homo sapiens] |
| 273 | 394121.2 | 2796118 | g3046817 | 1.00E−117 | glutathione-requiring prostaglandin D synthase [Homo sapiens] |
| 274 | 2347046CB1 | 2347046 | g443669 | 1.00E−117 | protein phosphatase [Homo sapiens] |
| 275 | 1137894.1 | 2059420 | g183445 | 1.00E−116 | zinc finger transcriptional regulator [Homo sapiens] |
| 276 | 1137536.4 | 1753015 | g2674061 | 1.00E−115 | Homo sapiens 3-phosphoglycerate dehydrogenase mRNA, complete cds. |
| 277 | 2501808CB1 | 2501808 | g7416120 | 1.00E−114 | ubiquitin-conjugating enzyme isolog [Homo sapiens] |
| 278 | 4767318CB1 | 3088261 | g7299015 | 1.00E−112 | CG9615 gene product [Drosophila melanogaster] |
| 279 | 1223705CB1 | 1633118 | g579930 | 1.00E−109 | glutathione peroxidase-GI [Homo sapiens] |
| 280 | 296696.7 | 851875 | g5725508 | 1.00E−108 | METH2 protein [Homo sapiens] |
| 281 | 410320.1 | 2886536 | g1033887 | 1.00E−107 | H. sapiens CpG island DNA genomic Mse1 fragment, |
| | | | | | clone 53d4, reverse read cpg53d4.rt1b. |
| 282 | 426109.1 | 2057823 | g181916 | 1.00E−107 | ubiquitin carrier protein [Homo sapiens] |
| 283 | 1098589.27 | 2152363 | g35068 | 1.00E−105 | Nm23 protein [Homo sapiens] |
| 284 | 233575.1 | 1222942 | g6177738 | 1.00E−104 | MCT-1 [Homo sapiens] |
| 285 | 235191.4 | 1997915 | g2062373 | 1.00E−101 | cyclin-selective ubiquitin carrier protein [Homo sapiens] |
| 286 | 468221.19 | 1662856 | g1469920 | 1.00E−100 | D53 [Homo sapiens] |
| 287 | 986342.1 | 2811372 | g4678526 | 5.00E−99 | dJ971N18.2 [Homo sapiens‘] |
| 288 | 240120.3 | 2795141 | g3094994 | 2.00E−95 | HBGF [Homo sapiens] |
| 289 | 5511889CB1 | 6105902 | g2612868 | 2.00E−94 | down syndrome candidate region 1; |
| | | | | | one of four alternatively spliced exon 1 [Homo sapiens] |
| 290 | 3009578CB1 | 3009578 | g1665817 | 4.00E−94 | Similar to S. cerevisiae hypothetical protein L3111 (S59316) [Homo sapiens[ |
| 291 | 982520.1 | 4821815 | g7573532 | 4.00E−93 | dJ136014.2 (collagen, type X, alpha 1) [Homo sapiens] |
| 292 | 899156.36 | 2851850 | g29904 | 3.00E−92 | cfos [Homo sapiens] |
| 293 | 1748428CB1 | 1658215 | g37481 | 3.00E−92 | TTG-2a/RBTN-2a [Homo sapiens] |
| 294 | 238540.1 | 1608244 | g5103021 | 3.00E−91 | Homo sapiens genomic DNA, chromosome 22q11.2, clone N110F4. |
| 295 | 898495.3 | 5261507 | g1575365 | 1.00E−90 | Human DSS1 pseudogene (DSS1P1), complete sequence. |
| 296 | 369213.48 | 1292449 | g579592 | 9.00E−90 | alpha 2-macroglobulin 690-730 [Homo sapiens] |
| 297 | 3084563CB1 | 5834427 | g181071 | 2.00E−89 | cysteine-rich protein [Homo sapiens] |
| 298 | 237613.7 | 1975550 | g7292879 | 2.00E−89 | CG1998 gene product [Drosophila melanogaster] |
| 299 | 2630652CB1 | 2152363 | g35068 | 2.00E−87 | Nm23 protein [Homo sapiens] |
| 300 | 5785224GB1 | 1212335 | g7300628 | 9.00E−87 | CG10877 gene product [Drosophila melanogaster] |
| 301 | 409911.8 | 2219364 | g2474096 | 2.00E−86 | XMP [Homo sapiens] |
| 302 | 2418523CB1 | 2454639 | g191983 | 4.00E−86 | clathrin-associated protein 19 [Mus musculus] |
| 303 | 1091297.30 | 2418490 | g189226 | 6.00E−86 | putative [Homo sapiens] |
| 304 | 369213.2 | 2200842 | g6048565 | 1.00E−85 | retinoid inducible gene 1 [Homo sapiens] |
| 305 | 221869.5 | 5523515 | g7243133 | 2.00E−82 | KIAA1376 protein [Homo sapiens] |
| 306 | 995529.6 | 2957476 | g29839 | 1.00E−81 | CDC2 polypeptide (CDC2) (AA 1-297) [Homo sapiens] |
| 307 | 982959.1 | 3115823 | g4028581 | 1.00E−81 | connective tissue growth factor related protein WISP-1 [Homo sapiens] |
| 308 | 342074.1 | 4408943 | g5732680 | 1.00E−81 | amino acid transporter B0+ [Homo sapiens] |
| 309 | 245194.2 | 1512826 | g2920504 | 2.00E−81 | frpHE [Homo sapiens] |
| 310 | 246444.6 | 3382391 | g6688153 | 1.00E−78 | small proline-rich protein 3 [Homo sapiens] |
| 311 | 374054.4 | 3843227 | g7301679 | 3.00E−78 | CG1540 gene product [Drosophila melanogaster] |
| 312 | 001322.4 | 2663164 | g7243009 | 1.00E−77 | KIAA1314 protein [Homo sapiens] |
| 313 | 406438.12 | 2797787 | g1552327 | 5.00E−76 | H. sapiens mRNA for TFG protein. |
| 314 | 1382961.12 | 4382348 | g34075 | 1.00E−75 | keratin related product [Homo sapiens] |
| 315 | 1359783CB1 | 3138721 | g458228 | 5.00E−75 | extracellular protein [Homo sapiens] |
| 316 | 1796035CB1 | 3986547 | g641937 | 1.00E−74 | ribonuclease A precursor [Homo sapiens] |
| 317 | 444850.11 | 2860704 | g7023332 | 4.00E−73 | unnamed protein product [Homo sapiens] |
| 318 | 236062CB1 | 2238411 | g6434876 | 4.00E−72 | CDK4-binding protein p345EI1 [Homo sapiens] |
| 319 | 333238.12 | 2833637 | g7021853 | 5.00E−72 | unnamed protein product [Homo sapiens] |
| 320 | 475547.2 | 1955363 | g7023806 | 2.00E−71 | unnamed protein product [Homo sapiens] |
| 321 | 977929.1 | 4112181 | g6690339 | 3.00E−71 | hematopoietic zinc finger protein [Mus musculus] |
| 322 | 1362715CB1 | 1816113 | g6457338 | 1.00E−69 | E2IG1 [Homo sapiens] |
| 323 | 3117642CB1 | 3117642 | g3300092 | 4.00E−69 | prostate associated PAGE−1 [Homo sapiens] |
| 324 | 2026270CB1 | 2026270 | g190726 | 9.00E−68 | parathyroid hormone-related protein precursor [Homo sapiens] |
| 325 | 981662.2 | 4401727 | g7022306 | 3.00E−67 | unnamed protein product [Homo sapiens] |
| 326 | 453004.10 | 2394888 | g4704752 | 6.00E−67 | calpain 3; calcium activated neutral protease; CAPN3; CL1 [Homo sapiens] |
| 327 | 978147.7 | 2784394 | g7022973 | 5.00E−66 | unnamed protein product [Homo sapiens] |
| 328 | 2132626CB1 | 541875 | g3171914 | 6.00E−66 | RAMP3 [Homo sapiens] |
| 329 | 402716.37 | 4199466 | g3582143 | 1.00E−65 | DNA-binding zinc finger(GBF) [Homo sapiens] |
| 330 | 464482.1 | 625374 | g181227 | 3.00E−65 | cytochrome b5 [Homo sapiens] |
| 331 | 402716.20 | 4199466 | g2745961 | 1.00E−64 | Bcd orf2 [Homo sapiens] |
| 332 | 1137710.5 | 1713191 | g471126 | 4.00E−64 | Id-2H [Homo sapiens] |
| 333 | 348912.3 | 1716655 | g219936 | 2.00E−62 | NCA-W272 [Homo sapiens] |
| 334 | 474926.11 | 2512203 | g312334 | 1.00E−61 | macrophage migration inhibitory factor [Homo sapiens] |
| 335 | 406804.4 | 3130454 | g6331328 | 1.00E−61 | KIAA1280 protein [Homo sapiens] |
| 336 | 480855.1 | 3234063 | g6808254 | 1.00E−60 | hypothetical protein [Homo sapiens] |
| 337 | 238593.5 | 211779 | g7106770 | 3.00E−60 | HSPC190 [Homo sapiens] |
| 338 | 373514.7 | 4740251 | g8515711 | 3.00E−60 | EXP35 [Homo sapiens] |
| 339 | 1383354.10 | 5057204 | g6983729 | 4.00E−60 | dJ977B1.5 (myosin regulatory light chain 2, |
| | | | | | smooth muscle isoform) [Homo sapiens] |
| 340 | 3120070CB1 | 3120070 | g7582391 | 4.00E−57 | p53 apoptosis-associated target [Mus musculus] |
| 341 | 253987.19 | 2232658 | g395338 | 2.00E−55 | helix-loop-helix protein [Homo sapiens] |
| 342 | 133425.16 | 3510656 | g178349 | 2.00E−54 | fructose 1,6-bisphosphatase (EC 3.1.3.11) [Homo sapiens] |
| 343 | 468221.18 | 1662856 | g1469920 | 5.00E−54 | D53 [Homo sapiens] |
| 344 | 020093.8 | 2102756 | g573114 | 6.00E−54 | Clq B-chain precursor [Homo sapiens] |
| 345 | 1556751CB1 | 1986121 | g7959303 | 1.00E−52 | KIAA1518 protein [Homo sapiens] |
| 346 | 1397976.1 | 4628258 | g306799 | 2.00E−52 | pregnancy-specific beta-glycoprotein e [Homo sapiens] |
| 347 | 233828.16 | 1362831 | g7021111 | 2.00E−52 | unnamed protein Product [Homo sapiens] |
| 348 | 1253414CB1 | 5681633 | g450281 | 6.00E−52 | isolog of yeast sui1 and rice gos2; putative [Homo sapiens] |
| 349 | 1101068.1 | 5856402 | g6164743 | 4.00E−51 | F-box protein Fbx20 [Homo sapiens] |
| 350 | 006922.1 | 2934515 | g7242957 | 4.00E−49 | KIAA 1301 protein [Homo sapiens] |
| 351 | 333398.5 | 2456903 | g6502523 | 2.00E−48 | Smad6 protein [Homo sapiens] |
| 352 | 235725.21 | 2095728 | g2407068 | 3.00E−48 | TFAR19 [Homo sapiens] |
| 353 | 242472.14 | 4572916 | g34416 | 1.00E−47 | precursor (AA −19 to 692) [Homo sapiens] |
| 354 | 253550.20 | 3397390 | g183116 | 3.00E−46 | insulin-like growth factor-binding protein [Homo sapiens] |
| 355 | 216262.3 | 3813934 | g4128051 | 5.00E−46 | EBI1-31 ligand chemokine [Homo sapiens] |
| 356 | 235191.3 | 1997915 | g6706799 | 5.00E−46 | dJ447F3.2 (ubiquitin-conjugating enzyme E2 H10) [Homo sapiens] |
| 357 | 480337.45 | 4602215 | g1167 | 4.00E−45 | cpn10 protein [Bos taurus] |
| 358 | 199939.6 | 1858415 | g2232019 | 8.00E−44 | HPV16 E1 protein binding protein [Homo sapiens] |
| 359 | 201204.9 | 4088394 | g4323528 | 1.00E−43 | cell cycle protein CDC20 [Homo sapiens] |
| 360 | 201887.2 | 3478024 | g2988398 | 2.00E−43 | Unknown gene product [Homo sapiens] |
| 361 | 1136056.1 | 3527982 | g1082038 | 3.00E−43 | G053 is human homolog of mouse FOSB gene [Homo sapiens] |
| 362 | 994977.1 | 3722056 | g30102 | 4.00E−43 | type I collagen [Homo sapiens] |
| 363 | 888669.8 | 4721466 | g7329217 | 6.00E−42 | TS58 [Homo sapiens] |
| 364 | 345860.20 | 5160686 | g29710 | 9.00E−42 | preprocathepsin H (AA −22 to 314) [Homo sapiens] |
| 365 | 480337.43 | 1459082 | g6996446 | 2.00E−39 | chaperonin 10, Hsp10 protein [Homo sapiens] |
| 365 | 480337.43 | 4602215 | g6996446 | 2.00E−39 | chaperonin 10, Hsp10 protein [Homo sapiens] |
| 366 | 399300.14 | 2842978 | g3818590 | 2.00E−38 | alpha-catenin-like protein; CG-4 [Homo sapiens] |
| 367 | 368015.2 | 3115792 | g7717449 | 3.00E−38 | Homo sapiens chromosome 21 segment HS21C103. |
| 368 | 227550.1 | 3771020 | g4028563 | 7.00E−38 | brain and nasopharyngeal carcinoma susceptibility protein |
| | | | | | NSG-x [Homo sapiens] |
| 369 | 201906.6 | 3733666 | g7416858 | 2.00E−36 | MBIP [Homo sapiens] |
| 370 | 349589.10 | 127321 | g2370126 | 6.00E−36 | LIM-31 domain protein [Homo sapiens] |
| 371 | 3713867CB1 | 2061401 | g28608 | 4.00E−35 | precursor polypeptide (AA −36 to 479) [Homo sapiens] |
| 372 | 235943.27 | 2957567 | g188870 | 1.00E−34 | polymorphic epithelial mucin [Homo sapiens] |
| 373 | 241742.1 | 1295905 | g190168 | 2.00E−34 | Homo sapiens dinucleotide repeat polymorphism, at locus D5S178 |
| 374 | 333680.1 | 3138456 | g7295285 | 4.00E−34 | melt gene product [Drosophila melanogaster] |
| 375 | 411429.3 | 1424886 | g4929719 | 9.00E−34 | CGI-125 protein [Homo sapiens] |
| 376 | 2356055CB1 | 2356055 | g6580815 | 9.00E−34 | indolethylamine N-methyltransferase [Homo sapiens] |
| 377 | 239579.3 | 1704713 | g3462455 | 2.00E−33 | junctional adhesion molecule [Mus musculus] |
| 378 | 332240.1 | 2201507 | g7022637 | 3.00E−32 | unnamed protein product [Homo sapiens] |
| 379 | 255161.1 | 2270986 | g7107421 | 3.00E−32 | ferritin light chain [Cavia porcellus] |
| 380 | 2454384CB1 | 2454384 | g1293145 | 2.00E−31 | rhotekin [Mus musculus] |
| 381 | 1383298.3 | 1510539 | g1711117 | 6.00E−31 | ligand activated transcription factor PPARgamma2 [Homo sapiens] |
| 382 | 383376.7 | 5551761 | g340361 | 1.00E−30 | von Willebrand factor prepropeptide [Homo sapiens] |
| 383 | 230816.1 | 1526322 | g7340847 | 2.00E−29 | chondroItin 4-sulfotransferase [Mus musculus] |
| 384 | 211949.2 | 1966295 | g5817053 | 3.00E−29 | hypothetical protein [Homo sapiens] |
| 385 | 238853.42 | 1450886 | g386803 | 2.00E−28 | 40-kDa keratin protein [Homo sapiens] |
| 386 | 333165.2 | 1682337 | g7020625 | 6.00E−28 | unnamed protein product [Homo sapiens] |
| 387 | 344868.12 | 3333118 | g4761222 | 1.00E−27 | Homo sapiens NADP+-dependent isocitrate |
| | | | | | dehydrogenase (PICD) mRNA, complete cds. |
| 388 | 148304.14 | 3646303 | g8131858 | 1.00E−27 | putative thymic stromal co-transporter TSCOT [Mus musculus] |
| 389 | 247747.6 | 3124204 | g4377993 | 6.00E−27 | tumor transforming protein 1 [Homo sapiens] |
| 390 | 001153.12 | 5546984 | g505033 | 4.00E−26 | mitogen inducible gene mig-2 [Homo sapiens] |
| 391 | 1095728.19 | 414480 | g7209525 | 1.00E−25 | DRAL/Slim3/FHL2 [Homo sapiens] |
| 392 | 222604.7 | 2745735 | g7293742 | 4.00E−25 | CG15881 gene product [Drosophila melanogaster] |
| 393 | 1094984.14 | 2620487 | g6330840 | 9.00E−25 | KIAA1247 protein [Homo sapiens] |
| 394 | 400702.1 | 1969974 | g7770167 | 9.00E−25 | PRO2176 [Homo sapiens] |
| 395 | 196557.1 | 1927026 | g2570154 | 3.00E−24 | 17-kDa PKC-potentiated inhibitory protein of PP1 [Sus scrofa] |
| 396 | 237208.5 | 993365 | g8037909 | 6.00E−24 | PAR6A [Mus musculus] |
| 397 | 337792.2 | 1832594 | g219476 | 1.00E−23 | APR peptide [Homo sapiens] |
| 398 | 398970.3 | 4885619 | g387015 | 1.00E−23 | pepsinogen C [Homo sapiens] |
| 399 | 1309633.3 | 1880421 | g7717462 | 9.00E−23 | AgX-1 antigen [Homo sapiens] |
| 400 | 981662.1 | 4401727 | g7022306 | 4.00E−21 | unnamed protein product [Homo sapiens] |
| 401 | 081187.1 | 2416447 | g219928 | 9.00E−21 | Human midkine gene, complete cds. |
| 402 | 086533.6 | 219839 | g533381 | 1.00E−19 | homologous to members of the I-kappa B family; |
| | | | | | protein binds NF-kappa B proteins [Homo sapiens] |
| 403 | 1093481.2 | 1962139 | g1814277 | 2.00E−19 | A33 antigen precursor [Homo sapiens] |
| 404 | 024494.7 | 1938744 | g6970439 | 2.00E−19 | CLST 11240 protein [Homo sapiens] |
| 405 | 888669.7 | 4721466 | g337451 | 3.00E−19 | hnRNP type A/B protein [Homo sapiens] |
| 406 | 428206.1 | 2675284 | g7291878 | 3.00E−19 | CG2811 gene product [Drosophila melanogaster] |
| 407 | 1328026.7 | 549728 | g3065741 | 4.00E−18 | small proline-rich protein 1A [Mus musculus] |
| 408 | 475113.7 | 1700462 | g7270660 | 5.00E−18 | putative protein [Arabidopsis thaliana] |
| 409 | 204392.5 | 2950394 | g510339 | 6.00E−18 | phosphatidylethanolamine-binding protein [Rattus norvegicus] |
| 410 | 1092257.12 | 3440567 | g1857161 | 2.00E−17 | hEZF [Homo sapiens] |
| 411 | 1134990.3 | 3878116 | g4894950 | 7.00E−17 | encephalopsin [Mus musculus] |
| 412 | 1095223.13 | 4158982 | g1478205 | 1.00E−16 | PNG protein [Mus musculus] |
| 413 | 214654.1 | 1681727 | g7020625 | 4.00E−16 | unnamed protein product [Homo sapiens] |
| 414 | 086533.5 | 219839 | g179376 | 7.00E−16 | lymphoma 3-encoded protein (bcl-3) [Homo sapiens] |
| 415 | 474117.5 | 3992320 | g1620561 | 2.00E−15 | C-1 [Homo sapiens] |
| 416 | 410812.1 | 3380665 | g7768737 | 2.00E−15 | Homo sapiens genomic DNA, chromosome 21q, section 89/105. |
| 417 | 011822.2 | 2887646 | g2239242 | 5.00E−15 | kinesin-like protein [Schizosaccharomyces pombe] |
| 418 | 252899.7 | 2811710 | g7959267 | 2.00E−14 | KIAA1503 protein [Homo sapiens] |
| 419 | 1511488CB1 | 1511488 | g6457344 | 3.00E−14 | E2IG5 [Homo sapiens] |
| 420 | 1383797.1 | 1466844 | g2088550 | 5.00E−14 | hereditary haemochromatosis region [Homo sapiens] |
| 421 | 400745.1 | 4205017 | g7688148 | 3.00E−13 | hypothetical protein [Homo sapiens] |
| 422 | 029618.1 | 1223877 | g4678684 | 7.00E−13 | hypothetical protein [Schizosaccharomyces pombe] |
| 423 | 245722.8 | 2508079 | g1694828 | 1.00E−12 | S100 calcium-binding protein A13 (S100A13) [Homo sapiens] |
| 424 | 981488.1 | 2159213 | g7020292 | 1.00E−12 | unnamed protein product [Homo sapiens] |
| 424 | 981488.1 | 2449016 | g7020292 | 1.00E−12 | unnamed protein product [Homo sapiens] |
| 425 | 241858.1 | 4460483 | g7768730 | 1.00E−12 | Homo sapiens genomic DNA, chromosome 21q, section 73/105. |
| 426 | 1466276CB1 | 739191 | g63466 | 5.00E−12 | histone H2A [Gallus gallus] |
| 427 | 999386.3 | 5286390 | g7295892 | 8.00E−11 | CG3104 gene product [Drosophila melanogaster] |
| 428 | 1091079.8 | 1503624 | g1946351 | 1.00E−10 | cell surface protein HCAR [Homo sapiens] |
| 429 | 407084.1 | 3423374 | g177869 | 4.00E−10 | Human alpha-2-macroglobulin mRNA, complete cds. |
| 430 | 215642.2 | 1923709 | g6331286 | 5.00E−10 | Homo sapiens mRNA for KIAA1274 protein, |
| | | | | | partial cds. |
| 431 | 1159769.1 | 3929017 | g6630766 | 6.00E−10 | dJ300I2.1 (secretory leukocyte protease inhibitor |
| | | | | | (antileukoproteinase)) [Homo sapiens] |
| 432 | 482336.31 | 4104390 | g1200072 | 2.00E−09 | keratin [Homo sapiens] |
| 433 | 064703.1 | 1900111 | g5712737 | 6.00E−09 | Homo sapiens alpha-1-antitrypsin nuclear matrix attachment region sequence. |
| 434 | 294837.1 | 3255489 | g1054740 | 1.00E−08 | chromosomal region [Homo sapiens] |
| 435 | 982699.1 | 5394922 | g1438107 | 1.00E−08 | Homo sapiens DNA for NRAMP1, partial cds. |
| 436 | 015126.1 | 4361169 | g5103017 | 1.00E−08 | Homo sapiens genomic DNA, chromosome 22q11.2, clone KB1674E1. |
| 437 | 235132.10 | 5682290 | g7573229 | 1.00E−08 | profilin II [Mus musculus] |
| 438 | 1070092.1 | 2965648 | g4927774 | 4.00E−08 | HHLA3 protein [Homo sapiens] |
| 439 | 272599.1 | 2278772 | g5926699 | 4.00E−08 | Homo sapiens genomic DNA, chromosome 6p21.3, |
| | | | | | HLA Class I region, section 11/20 |
| 440 | 415901.1 | 2998131 | g1203968 | 1.00E−07 | chromosome X region from filamin (FLN) gene to |
| | | | | | glucose-6-phosphate dehydrogenase (G6PD) gene [Homo sa |
| 441 | 477387.3 | 3100048 | g469478 | 2.00E−07 | SM-20 [Rattus norvegicus] |
| 442 | 208702.1 | 3658444 | g6969578 | 2.00E−07 | Homo sapiens glutaminase kidney isoform mRNA, complete cds. |
| 443 | 1135039.1 | 899118 | g7020625 | 4.00E−07 | unnamed protein product [Homo sapiens] |
| 444 | 232649.2 | 2234640 | g7768718 | 8.00E−07 | Homo sapiens genomic DNA, chromosome 21q, section 62/105. |
| 445 | 340450.1 | 4639995 | g558908 | 9.00E−07 | reverse transcriptase [Mus musculus] |
| 446 | 320660.1 | 5034026 | g7768679 | 9.00E−07 | Homo sapiens genomic DNA, chromosome 21q, section 64/105. |
| 447 | 467104.21 | 2054055 | g397606 | 2.00E−06 | H. sapiens encoding CLA-1 mRNA. |
| 448 | 332382.1 | 3999569 | g4240144 | 4.00E−06 | Homo sapiens mRNA for KIAA0828 protein, partial cds. |
| 449 | 2104530CB1 | 2104530 | g806564 | 1.00E−04 | Sm protein F [Homo sapiens] |
| 450 | 007228.1 | 60306 | | 0 | Incyte Unique |
| 451 | 008098.1 | 313312 | | 0 | Incyte Unique |
| 452 | 016124.2 | 3732960 | | 0 | Incyte Unique |
| 453 | 016149.1 | 3736058 | | 0 | Incyte Unique |
| 454 | 025731.1 | 2586554 | | 0 | Incyte Unique |
| 455 | 026856.1 | 1300730 | | 0 | Incyte Unique |
| 456 | 028918.1 | 2800380 | | 0 | Incyte Unique |
| 457 | 032481.1 | 4073339 | | 0 | Incyte Unique |
| 458 | 045630.1 | 1269876 | | 0 | Incyte Unique |
| 459 | 047533.1 | 4276910 | | 0 | Incyte Unique |
| 460 | 072540.1 | 2152929 | | 0 | Incyte Unique |
| 461 | 076580.1 | 2264967 | | 0 | Incyte Unique |
| 462 | 1072776.1 | 5505238 | | 0 | Incyte Unique |
| 463 | 1100233.1 | 3725970 | | 0 | Incyte Unique |
| 464 | 1102220.2 | 1299351 | | 0 | Incyte Unique |
| 465 | 1328508.1 | 2669974 | | 0 | Incyte Unique |
| 466 | 1383457.1 | 5024278 | | 0 | Incyte Unique |
| 467 | 140230.5 | 1211835 | | 0 | Incyte Unique |
| 468 | 150868.1 | 491129 | | 0 | Incyte Unique |
| 469 | 172582.1 | 5622463 | | 0 | Incyte Unique |
| 470 | 175536.1 | 122943 | | 0 | Incyte Unique |
| 470 | 175536.1 | 5187276 | | 0 | Incyte Unique |
| 471 | 176928.1 | 2782090 | | 0 | Incyte Unique |
| 472 | 197252.1 | 2656358 | | 0 | Incyte Unique |
| 473 | 1975480CB1 | 3852527 | | 0 | Incyte Unique |
| 474 | 200578.1 | 473724 | | 0 | Incyte Unique |
| 475 | 202117.4 | 1402988 | | 0 | Incyte Unique |
| 476 | 204750.3 | 2021485 | | 0 | Incyte Unique |
| 477 | 205232.1 | 1379153 | | 0 | Incyte Unique |
| 478 | 212157.1 | 3130003 | | 0 | Incyte Unique |
| 479 | 221846.4 | 4116336 | | 0 | Incyte Unique |
| 480 | 222092.1 | 2154847 | | 0 | Incyte Unique |
| 481 | 228350.1 | 2605849 | | 0 | Incyte Unique |
| 482 | 229170.1 | 1818484 | | 0 | Incyte Unique |
| 483 | 230820.1 | 3120378 | | 0 | Incyte Unique |
| 484 | 231160.1 | 1848676 | | 0 | Incyte Unique |
| 485 | 232212.1 | 1866738 | | 0 | Incyte Unique |
| 486 | 234090.1 | 1718918 | | 0 | Incyte Unique |
| 487 | 236432.1 | 3322032 | | 0 | Incyte Unique |
| 488 | 236497.1 | 1682609 | | 0 | Incyte Unique |
| 489 | 237208.4 | 993365 | | 0 | Incyte Unique |
| 490 | 2484813CB1 | 1338090 | | 0 | Incyte Unique |
| 491 | 249104.1 | 2394947 | | 0 | Incyte Unique |
| 492 | 287241.1 | 3412108 | | 0 | Incyte Unique |
| 493 | 316571.1 | 5185101 | | 0 | Incyte Unique |
| 494 | 331447.1 | 3363538 | | 0 | Incyte Unique |
| 495 | 333127.1 | 153430 | | 0 | Ineyte Unique |
| 496 | 334025.5 | 3724351 | | 0 | Incyte Unique |
| 497 | 334570.1 | 1296146 | | 0 | Incyte Unique |
| 498 | 334814.1 | 1645745 | | 0 | Incyte Unique |
| 499 | 335692.1 | 5023108 | | 0 | Incyte Unique |
| 500 | 337407.1 | 3518117 | | 0 | Incyte Unique |
| 501 | 337953.2 | 1378835 | | 0 | Incyte Unique |
| 502 | 337953.5 | 1378835 | | 0 | Incyte Unique |
| 503 | 346431.1 | 2723937 | | 0 | Incyte Unique |
| 504 | 368357.1 | 5085077 | | 0 | Incyte Unique |
| 505 | 390546.1 | 5339122 | | 0 | Incyte Unique |
| 506 | 400267.1 | 4240272 | | 0 | Incyte Unique |
| 507 | 402288.1 | 3871646 | | 0 | Incyte Unique |
| 508 | 403407.2 | 1316042 | | 0 | Incyte Unique |
| 509 | 405447.1 | 3220727 | | 0 | Incyte Unique |
| 510 | 407485.1 | 5552541 | | 0 | Incyte Unique |
| 511 | 427832.19 | 3692235 | | 0 | Incyte Unique |
| 512 | 477913.1 | 2593092 | | 0 | Incyte Unique |
| 513 | 902956.2 | 2607572 | | 0 | Incyte Unique |
| 514 | 979367.4 | 2998372 | | 0 | Incyte Unique |
| 515 | 979367.5 | 2998372 | | 0 | Incyte Unique |
| 516 | 982861.1 | 3888854 | | 0 | Incyte Unique |
| 517 | 983351.1 | 1819244 | | 0 | Incyte Unique |
| 518 | 984637.1 | 1610772 | | 0 | Incyte Unique |
| 519 | 984900.1 | 1802185 | | 0 | Incyte Unique |

TABLE 3

| SEQ ID NO | Template ID | Clone ID | Start | Stop |
|---|---|---|---|---|
| 1 | 1867417CB1 | 1357231 | 3074 | 3751 |
| 2 | 1970111CB1 | 1970111 | 1059 | 2805 |
| 3 | 959142CB1 | 1930447 | 4830 | 5369 |
| 4 | 064684.7 | 1824717 | 9 | 655 |
| 5 | 245093.34 | 958486 | 2723 | 3083 |
| 6 | 331908.5 | 1995457 | 3146 | 3716 |
| 7 | 1329880.35 | 5150602 | 3980 | 4759 |
| 8 | 963555.1 | 2820294 | 3306 | 3835 |
| 9 | 199471.2 | 2047549 | 148 | 1452 |
| 10 | 048849.1 | 1358605 | 243 | 654 |
| 11 | 278283.1 | 4180161 | 1 | 85 |
| 12 | 978433CB1 | 1869068 | 513 | 903 |
| 13 | 611514CB1 | 605219 | 1131 | 1679 |
| 14 | 1382907.35 | 1888708 | 6880 | 7488 |
| 15 | 350509.2 | 1510413 | 5890 | 6572 |
| 16 | 2512879CB1 | 2512879 | 130 | 1418 |
| 17 | 241123.1 | 3876732 | 667 | 1255 |
| 18 | 247817.4 | 1480063 | 4916 | 5331 |
| 19 | 1674368CB1 | 1241484 | 3525 | 3926 |
| 20 | 343963.1 | 2671006 | 3173 | 3987 |
| 21 | 273154CB1 | 2899786 | 13 | 4831 |
| 22 | 331508.4 | 1967759 | 3466 | 4070 |
| 23 | 1137924.1 | 1480479 | 2178 | 2679 |
| 24 | 247168.4 | 2278925 | 835 | 1671 |
| 25 | 2275817CB1 | 224996 | 1690 | 2282 |
| 26 | 2717806CB1 | 4918603 | 81 | 2260 |
| 27 | 407624.1 | 3771805 | 1 | 1009 |
| 28 | 3950154CB1 | 2748163 | 3041 | 3582 |
| 29 | 3741842CB1 | 1988080 | 3263 | 3973 |
| 30 | 221055.4 | 2718391 | 2218 | 2672 |
| 31 | 379425CB1 | 433622 | 1203 | 1858 |
| 32 | 1685090CB1 | 2721792 | 51 | 503 |
| 33 | 350476.1 | 123312 | 2919 | 4276 |
| 33 | 350476.1 | 4044520 | 3335 | 4279 |
| 33 | 350476.1 | 123312 | 2919 | 4276 |
| 33 | 350476.1 | 4044520 | 3335 | 4279 |
| 34 | 404278.1 | 3116479 | 1233 | 1641 |
| 35 | 1250492CB1 | 1361644 | 1420 | 1954 |
| 36 | 200095.2 | 3125685 | 498 | 945 |
| 37 | 997405.3 | 1240444 | 419 | 810 |
| 38 | 235369.10 | 1965041 | 3846 | 4581 |
| 39 | 236587.4 | 1362125 | 2251 | 2715 |
| 40 | 1135936.1 | 4271973 | 661 | 1039 |
| 41 | 251123.6 | 3557441 | 1451 | 1951 |
| 41 | 251123.6 | 2600963 | 236 | 1946 |
| 41 | 251123.6 | 3557441 | 1451 | 1951 |
| 41 | 251123.6 | 2600963 | 236 | 1946 |
| 42 | 2547002CB1 | 2475740 | 1 | 1339 |
| 43 | 251123.8 | 3557441 | 949 | 1356 |
| 44 | 1000172.35 | 3645309 | 1 | 362 |
| 45 | 2921920CB1 | 1498363 | 520 | 1103 |
| 46 | 631645CB1 | 1274935 | 67 | 626 |
| 46 | 631645CB1 | 1274935 | 67 | 626 |
| 46 | 631645CB1 | 1577756 | 27 | 412 |
| 47 | 1507546CB1 | 3507734 | 904 | 1196 |
| 48 | 1508437CB1 | 1376121 | 322 | 959 |
| 48 | 1508437CB1 | 1607091 | 220 | 959 |
| 48 | 1508437CB1 | 1376121 | 322 | 959 |
| 48 | 1508437CB1 | 1607091 | 220 | 959 |
| 49 | 1100669.4 | 1675122 | 467 | 972 |
| 50 | 124600CB1 | 1578941 | 38 | 611 |
| 51 | 470771.11 | 322066 | 706 | 1444 |
| 52 | 273259.4 | 2201411 | 1 | 565 |
| 53 | 5170638CB1 | 4014022 | 77 | 743 |
| 54 | 1330170.3 | 4221057 | 31 | 304 |
| 55 | 1383898.2 | 1499549 | 1 | 620 |
| 56 | 019238.3 | 1430507 | 728 | 1579 |
| 57 | 1102296.5 | 3026658 | 15 | 288 |
| 58 | 995673.3 | 2149968 | 314 | 862 |
| 58 | 995673.3 | 4215545 | 322 | 837 |
| 58 | 995673.3 | 2149968 | 314 | 862 |
| 58 | 995673.3 | 4215545 | 322 | 837 |
| 59 | 312256CB1 | 313697 | 1014 | 1463 |
| 60 | 199183.2 | 2763310 | 18 | 700 |
| 61 | 289671.40 | 3678546 | 1 | 506 |
| 62 | 154403.1 | 2050104 | 82 | 202 |
| 63 | 086518.22 | 1987759 | 175 | 393 |
| 64 | 1383156.10 | 311197 | 59 | 724 |
| 65 | 1100669.3 | 1675122 | 119 | 356 |
| 66 | 1330148.1 | 3867795 | 1 | 136 |
| 67 | 1436702CB1 | 1358185 | 907 | 2847 |
| 68 | 3141226CB1 | 3625857 | 725 | 1161 |
| 69 | 289671.44 | 970905 | 801 | 1217 |
| 70 | 197927.7 | 2417149 | 4 | 392 |
| 71 | 1134834.2 | 1911306 | 1 | 389 |
| 72 | 200273.1 | 2721144 | 19 | 775 |
| 73 | 477974.1 | 3117677 | 1 | 231 |
| 74 | 235095.7 | 1809377 | 826 | 1247 |
| 75 | 407593.1 | 205053 | 1020 | 1277 |
| 76 | 332301.1 | 1417150 | 645 | 1230 |
| 77 | 407699.4 | 1919860 | 1546 | 1959 |
| 78 | 405501.1 | 2593077 | 270 | 755 |
| 79 | 482541.2 | 2685454 | 900 | 1217 |
| 80 | 237026.3 | 2963374 | 111 | 1470 |
| 81 | 443605.4 | 3556587 | 359 | 947 |
| 82 | 443605.15 | 3556587 | 491 | 680 |
| 83 | 029997.1 | 3638532 | 1 | 489 |
| 84 | 331743.9 | 789903 | 6102 | 6915 |
| 85 | 337334.1 | 3220029 | 4222 | 4954 |
| 86 | 410438.2 | 1879818 | 2054 | 2460 |
| 87 | 453004.32 | 2394888 | 2871 | 3305 |
| 88 | 236319.2 | 3044230 | 1282 | 3990 |
| 89 | 978118.5 | 2508618 | 66 | 1524 |
| 90 | 1383177.16 | 1646505 | 3022 | 3654 |
| 91 | 1383298.1 | 1510539 | 1403 | 1763 |
| 92 | 1000222.20 | 1729693 | 1008 | 1319 |
| 93 | 1096863.27 | 1303863 | 3824 | 5091 |
| 94 | 245334.1 | 2380412 | 1301 | 1932 |
| 95 | 1656674CB1 | 2222802 | 1173 | 1622 |
| 96 | 522678CB1 | 2199851 | 1804 | 2300 |
| 97 | 1424985CB1 | 2150615 | 957 | 1509 |
| 98 | 1468237CB1 | 1573505 | 451 | 2131 |
| 99 | 337008.1 | 1599272 | 1108 | 1571 |
| 100 | 444850.9 | 2860704 | 2676 | 2975 |
| 101 | 786284CB1 | 1458210 | 1507 | 1881 |
| 102 | 1352170CB1 | 1988092 | 2751 | 3158 |
| 103 | 1815320CB1 | 1742116 | 2617 | 3234 |
| 104 | 331192.11 | 2007554 | 1584 | 2105 |
| 105 | 344053.5 | 2928545 | 1 | 487 |
| 106 | 411188.2 | 4247796 | 3887 | 4272 |
| 107 | 331510.4 | 3814138 | 5491 | 6057 |
| 108 | 492750CB1 | 2732630 | 2415 | 3688 |
| 109 | 348912.4 | 1716655 | 1033 | 1392 |
| 110 | 2680109CB1 | 2125020 | 51 | 2410 |
| 110 | 2680109CB1 | 2242677 | 2015 | 2150 |
| 110 | 2680109CB1 | 2125020 | 51 | 2410 |
| 110 | 2680109CB1 | 2242677 | 2015 | 2150 |
| 111 | 512261CB1 | 512261 | 1433 | 1788 |
| 112 | 232719.2 | 1848259 | 4889 | 5167 |
| 113 | 369664.2 | 3119171 | 142 | 551 |
| 114 | 021042.1 | 2879922 | −23 | 268 |
| 115 | 418805.7 | 1637320 | 7437 | 7843 |
| 116 | 1094000.4 | 1397816 | 1344 | 1667 |
| 117 | 1094000.5 | 1380927 | 854 | 1537 |
| 118 | 1222734CB1 | 1300701 | 1331 | 1717 |
| 118 | 1222734CB1 | 1397816 | 1342 | 1708 |
| 118 | 1222734CB1 | 1300701 | 1331 | 1717 |
| 118 | 1222734CB1 | 1397816 | 1342 | 1708 |
| 119 | 411205.5 | 1420883 | 2446 | 2877 |
| 120 | 1092777.6 | 1643711 | 148 | 635 |
| 121 | 234358.5 | 1996180 | 2099 | 2743 |
| 122 | 1092777.7 | 1643711 | 1193 | 1684 |
| 123 | 4180444CB1 | 3872317 | 5376 | 5711 |
| 124 | 1987983CB1 | 690819 | 213 | 519 |
| 125 | 1398420.2 | 4655050 | 6 | 1227 |
| 126 | 1749102CB1 | 1749102 | 51 | 1535 |
| 127 | 2965804GB | 12965804 | 249 | 1858 |
| 128 | 210095.21 | 1285926 | 894 | 1583 |
| 129 | 1555752CB1 | 42248 | 1956 | 2146 |
| 130 | 110245.1 | 3028719 | 1 | 396 |
| 131 | 009476CB1 | 2017386 | 1326 | 1690 |
| 132 | 1560874CB1 | 3815942 | 1230 | 1518 |
| 133 | 3571894CB1 | 2132715 | 3322 | 3854 |
| 134 | 349622.1 | 2055903 | 325 | 681 |
| 135 | 002940CB1 | 1679482 | 709 | 1223 |
| 136 | 1362466CB1 | 1834502 455 | 1509 | |
| 137 | 383376.19 | 4461157 | 428 | 997 |
| 138 | 1382961.3 | 3184882 | 1080 | 1401 |
| 139 | 1382961.1 | 54382348 | 1090 | 1204 |
| 140 | 1454852CB1 | 793403 | 891 | 1305 |
| 141 | 2161632CB1 | 954057 | 3078 | 3462 |
| 142 | 2058013CB1 | 1968413 | 2472 | 3001 |
| 143 | 235333.1 | 1684632 | 3737 | 4318 |
| 144 | 702628CB1 | 2418629 | 736 | 1832 |
| 145 | 1383415.3 | 1420380 | 2534 | 3186 |
| 146 | 236309.1 | 2925376 | 1420 | 1959 |
| 147 | 344868.13 | 3333118 | 1931 | 2322 |
| 148 | 368869.15 | 100602 | 1 | 821 |
| 149 | 3256566CB1 | 1666069 | 1777 | 2347 |
| 150 | 4104673CB1 | 4104673 | 1 | 1532 |
| 151 | 345511.2 | 4243318 | 111 | 462 |
| 152 | 1723834CB1 | 1723834 | 2901 | 3240 |
| 153 | 078756CB1 | 490795 | 2952 | 3202 |
| 154 | 331749.3 | 1628341 | 264 | 748 |
| 155 | 350528.7 | 1429293 | 2258 | 2819 |
| 156 | 1099159.1 | 1519404 | 192 | 406 |
| 157 | 1530186CB1 | 3715059 | 3278 | 4655 |
| 158 | 1092387.11 | 2674527 | 1078 | 1530 |
| 159 | 107939.2 | 2313581 | 2837 | 3302 |
| 160 | 899248.6 | 1832584 | 2558 | 3052 |
| 161 | 2959521CB1 | 87727 | 1538 | 1779 |
| 162 | 3331519CB1 | 2197965 | 3205 | 3861 |
| 163 | 1453334CB1 | 1445547 | 1416 | 2292 |
| 164 | 2798854CB1 | 4385292 | 1174 | 3091 |
| 165 | 227961.1 | 2742979 | 30 | 1490 |
| 166 | 1427470CB1 | 1430862 | 768 | 1588 |
| 167 | 206250.6 | 2180220 | 736 | 1789 |
| 168 | 995068.15 | 893230 | 468 | 1015 |
| 169 | 2545475CB1 | 1981569 | 2829 | 3230 |
| 170 | 1502559CB1 | 1502559 | 198 | 1580 |
| 171 | 2239738CB1 | 22397381 | 264 | 1909 |
| 172 | 347572.1 | 1501621 | 2094 | 3288 |
| 173 | 977985.10 | 1445387 | 15 | 583 |
| 174 | 236582.2 | 2720359 | 1 | 585 |
| 175 | 403676.1 | 1656490 | 1085 | 1990 |
| 176 | 138472CB1 | 2373085 | 1105 | 1780 |
| 177 | 253672.11 | 2851539 | 32 | 844 |
| 178 | 201928.3 | 2126712 | 2952 | 3429 |
| 179 | 198643.1 | 2187262 | 615 | 1263 |
| 180 | 979448.2 | 1497012 | 292 | 1817 |
| 181 | 460745.7 | 1381145 | 2892 | 3277 |
| 182 | 399300.18 | 2842978 | 2048 | 2428 |
| 183 | 248091.1 | 1607083 | 1228 | 1726 |
| 184 | 4030737CB1 | 2440848 | 1918 | 2439 |
| 185 | 3012575CB1 | 3012575 | 1372 | 2214 |
| 186 | 221827.1 | 2057406 | 59 | 513 |
| 187 | 238660.5 | 1604437 | 5456 | 5898 |
| 188 | 2171401CB1 | 2055814 | 854 | 1639 |
| 189 | 1420940CB1 | 1603408 | 1557 | 1775 |
| 190 | 1383160.14 | 1834906 | 53 | 610 |
| 191 | 233331.8 | 2154725 | 3246 | 3983 |
| 192 | 338217.10 | 1989545 | 2002 | 3687 |
| 193 | 5982278CB1 | 2640427 | 2624 | 3278 |
| 194 | 1097030.1 | 1760232 | 9300 | 10053 |
| 195 | 1100509.4 | 2215183 | 1705 | 2127 |
| 196 | 199671.1 | 1694119 | 2390 | 2978 |
| 197 | 232137.20 | 2053221 | 2799 | 3373 |
| 198 | 898608.2 | 3112520 | 1552 | 1669 |
| 199 | 411373.3 | 1625856 | 3912 | 4539 |
| 200 | 1092387.17 | 2070554 | 96 | 1273 |
| 201 | 201906.5 | 3733666 | 1140 | 1547 |
| 202 | 3607580CB1 | 3607580 | 2034 | 2829 |
| 203 | 991163CB1 | 1809178 | 933 | 1346 |
| 204 | 1309633.1 | 1880421 | 1868 | 2234 |
| 205 | 1384719.29 | 4372330 | 45 | 1713 |
| 206 | 178250.2 | 3075015 | 484 | 1011 |
| 207 | 1099945.13 | 1911742 | 5804 | 6313 |
| 208 | 2172334CB1 | 2172334 | 956 | 2250 |
| 209 | 253946.17 | 3625189 | 6204 | 6758 |
| 210 | 008513.49 | 2057510 | 1721 | 2258 |
| 211 | 1092257.2 | 1447795 | 1529 | 2772 |
| 211 | 1092257.2 | 1962235 | 2348 | 2796 |
| 211 | 1092257.2 | 3440567 | 2334 | 2777 |
| 211 | 1092257.2 | 1447795 | 1529 | 2772 |
| 211 | 1092257.2 | 1962235 | 2348 | 2796 |
| 211 | 1092257.2 | 3440567 | 2334 | 2777 |
| 211 | 1092257.2 | 1447795 | 1529 | 2772 |
| 211 | 1092257.2 | 1962235 | 2348 | 2796 |
| 211 | 1092257.2 | 3440567 | 2334 | 2777 |
| 212 | 4349106CB1 | 2820861 | 103 | 344 |
| 213 | 1814803CB1 | 1698951 | 1303 | 2084 |
| 214 | 1141764.8 | 1926114 | 1559 | 2161 |
| 215 | 406531.1 | 1603580 | 3326 | 3581 |
| 216 | 429307.4 | 3144021 | 1 | 374 |
| 217 | 239515.9 | 1675369 | 766 | 1201 |
| 218 | 1089210.1 | 5033671 | 34 | 1152 |
| 219 | 010796.18 | 1445895 | 879 | 1402 |
| 220 | 3752346CB1 | 1378037 | 577 | 1140 |
| 221 | 2729828CB1 | 1858171 | 1039 | 1673 |
| 222 | 1648579CB1 | 3142624 | 419 | 1638 |
| 223 | 1454418CB1 | 1468660 | 625 | 1167 |
| 224 | 995529.10 | 2957476 | 878 | 1254 |
| 225 | 2126751CB1 | 2126751 | 899 | 1528 |
| 226 | 569648CB1 | 2720467 | 526 | 1777 |
| 227 | 1352789CB1 | 3000146 | 47 | 872 |
| 228 | 1635966CB1 | 2046165 | 492 | 846 |
| 229 | 349415.6 | 3074415 | 3847 | 3974 |
| 230 | 406438.14 | 2797787 | 1529 | 2009 |
| 231 | 1471808CB1 | 3074415 | 1 | 501 |
| 232 | 245595.1 | 2052480 | 2023 | 2444 |
| 233 | 2784232CB1 | 3151158 | 1042 | 1764 |
| 234 | 2742913CB1 | 2516950 | 75 | 1693 |
| 235 | 1509972CB1 | 4047785 | 1133 | 1204 |
| 236 | 1097638.1 | 1903760 | 142 | 604 |
| 237 | 900035.58 | 1900306 | 786 | 6912 |
| 237 | 900035.58 | 5186773 | 5391 | 5851 |
| 237 | 900035.58 | 1900306 | 786 | 6912 |
| 237 | 900035.58 | 5186773 | 5391 | 5851 |
| 238 | 1075592.61 | 423848 | 4204 | 4771 |
| 239 | 984540.11 | 393337 | 1648 | 2198 |
| 240 | 378633.40 | 1821938 | 583 | 1119 |
| 241 | 477387.73 | 100048 | 1253 | 1700 |
| 242 | 347049.92 | 057158 | 2011 | 3007 |
| 243 | 1097717.18 | 5065213 | 346 | 1556 |
| 244 | 2832214CB1 | 2832214 | 6 | 1181 |
| 245 | 2947513CB1 | 5984038 | 22 | 502 |
| 246 | 1655369CB1 | 2056840 | 1867 | 2504 |
| 247 | 039170.3 | 2418484 | 106 | 532 |
| 248 | 1074926.1 | 2947513 | −11 | 292 |
| 249 | 344297.5 | 62144 | 721 | 908 |
| 250 | 410721.1 | 4688 | 1276 | 1548 |
| 251 | 1121097.1 | 1450641 | 2382 | 3709 |
| 252 | 1097450.22 | 3374194 | 55 | 301 |
| 253 | 238533.1 | 2790947 | 3027 | 3560 |
| 254 | 1097080.8 | 2694381 | 1400 | 1595 |
| 255 | 1097450.20 | 3374194 | 464 | 939 |
| 256 | 2514988CB1 | 1886886 | 809 | 1277 |
| 257 | 3130485CB1 | 1987238 | 407 | 1382 |
| 258 | 1330234.11 | 5392723 | 91 | 1528 |
| 259 | 1943624CB1 | 1943624 | 941 | 1440 |
| 260 | 2189816CB1 | 2189816 | 202 | 575 |
| 261 | 039027.1 | 3861522 | 1 | 223 |
| 262 | 2502336CB1 | 2507719 | 473 | 1659 |
| 263 | 3592543CB1 | 5066393 | 931 | 1312 |
| 264 | 399589.1 | 2070856 | 1574 | 2021 |
| 265 | 2175401CB1 | 1833174 | 763 | 1582 |
| 266 | 084455.9 | 1356268 | 1748 | 1920 |
| 267 | 233041.5 | 2060622 | 2750 | 3422 |
| 268 | 1382961.5 | 3184882 | 1 | 518 |
| 269 | 1518310CB1 | 1518310 | 1692 | 2154 |
| 270 | 1517817CB1 | 1402078 | 264 | 976 |
| 271 | 3090387CB1 | 1714684 | 747 | 1078 |
| 272 | 3836893CB1 | 3836893 | 365 | 881 |
| 273 | 394121.2 | 2796118 | 31 | 561 |
| 274 | 2347046CB1 | 2347046 | 7 | 847 |
| 275 | 1137894.1 | 2059420 | 1947 | 2552 |
| 276 | 1137536.4 | 1753015 | 189 | 531 |
| 277 | 2501808CB1 | 2501808 | 19 | 858 |
| 278 | 4767318CB1 | 3088261 | 961 | 1243 |
| 279 | 1223705CB1 | 1633118 | 504 | 986 |
| 280 | 296696.7 | 851875 | 203 | 1455 |
| 281 | 410320.1 | 2886536 | 1 | 546 |
| 282 | 426109.1 | 2057823 | 526 | 1043 |
| 283 | 1098589.27 | 2152363 | 348 | 729 |
| 284 | 233575.1 | 1222942 | 237 | 962 |
| 285 | 235191.4 | 1997915 | 213 | 782 |
| 286 | 468221.19 | 1662856 | 992 | 1313 |
| 287 | 986342.1 | 2811372 | 4490 | 4916 |
| 288 | 240120.3 | 2795141 | 572 | 1600 |
| 289 | 5511889CB1 | 6105902 | 1789 | 2336 |
| 290 | 3009578CB1 | 3009578 | 672 | 1239 |
| 291 | 982520.1 | 4821815 | 1559 | 2423 |
| 292 | 899156.36 | 2851850 | 1209 | 1525 |
| 293 | 1748428CB1 | 1658215 | 933 | 1395 |
| 294 | 238540.1 | 1608244 | 119 | 194 |
| 295 | 898495.3 | 5261507 | 1 | 581 |
| 296 | 369213.48 | 1292449 | 656 | 1542 |
| 297 | 3084563CB1 | 5834427 | 1436 | 1845 |
| 298 | 237613.7 | 1975550 | 2232 | 2858 |
| 299 | 2630652CB1 | 2152363 | 462 | 922 |
| 300 | 5785224CB1 | 1212335 | 931 | 1583 |
| 301 | 409911.8 | 2219364 | 246 | 989 |
| 302 | 2418523CB1 | 2454639 | 49 | 1172 |
| 303 | 1091297.30 | 2418490 | 168 | 635 |
| 304 | 369213.2 | 2200842 | 5 | 737 |
| 305 | 221869.5 | 5523515 | 70 | 357 |
| 306 | 995529.6 | 2957476 | 1993 | 2553 |
| 307 | 982959.1 | 3115823 | 2164 | 4269 |
| 308 | 342074.1 | 4408943 | 1 | 487 |
| 309 | 245194.2 | 1512826 | 709 | 1216 |
| 310 | 246444.6 | 3382391 | 20 | 853 |
| 311 | 374054.4 | 3843227 | 805 | 1333 |
| 312 | 001322.4 | 2663164 | 1069 | 3206 |
| 313 | 406438.12 | 2797787 | 313 | 502 |
| 314 | 1382961.12 | 4382348 | 1408 | 1786 |
| 315 | 1359783CB1 | 3138721 | 1545 | 2281 |
| 316 | 1796035CB1 | 3986547 | 84 | 524 |
| 317 | 444850.11 | 2860704 | 1 | 479 |
| 318 | 236062CB1 | 2238411 | 677 | 1143 |
| 319 | 333238.12 | 2833637 | 2398 | 2661 |
| 320 | 475547.2 | 1955363 | 2587 | 3041 |
| 321 | 977929.1 | 4112181 | 1192 | 1685 |
| 322 | 1362715CB1 | 1816113 | 61 | 531 |
| 323 | 3117642CB1 | 3117642 | 272 | 640 |
| 324 | 2026270CB1 | 2026270 | 532 | 800 |
| 325 | 981662.2 | 4401727 | 1 | 433 |
| 326 | 453004.10 | 2394888 | 1000 | 1393 |
| 327 | 978147.7 | 2784394 | 468 | 1899 |
| 328 | 2132626CB1 | 541875 | 724 | 1279 |
| 329 | 402716.37 | 4199466 | 1123 | 1384 |
| 330 | 464482.1 | 625374 | 20 | 811 |
| 331 | 402716.20 | 4199466 | 2052 | 2508 |
| 332 | 1137710.5 | 1713191 | −14 | 177 |
| 333 | 348912.3 | 1716655 | 1 | 533 |
| 334 | 474926.11 | 2512203 | 3202 | 3716 |
| 335 | 406804.4 | 3130454 | 1581 | 2299 |
| 336 | 480855.1 | 3234063 | 572 | 1000 |
| 337 | 238593.5 | 211779 | 675 | 1163 |
| 338 | 373514.7 | 4740251 | 1 | 508 |
| 339 | 1383354.10 | 5057204 | 6 | 470 |
| 340 | 3120070CB1 | 3120070 | 351 | 1931 |
| 341 | 253987.19 | 2232658 | 1707 | 2066 |
| 342 | 133425.16 | 3510656 | 174 | 1414 |
| 343 | 468221.18 | 1662856 | 257 | 765 |
| 344 | 020093.8 | 2102756 | 487 | 926 |
| 345 | 1556751CB1 | 1986121 | 635 | 1379 |
| 346 | 1397976.1 | 4628258 | 1 | 1534 |
| 347 | 233828.16 | 1362831 | 870 | 1224 |
| 348 | 1253414CB1 | 5681633 | 802 | 1177 |
| 349 | 1101068.1 | 5856402 | 399 | 859 |
| 350 | 006922.1 | 2934515 | 1 | 257 |
| 351 | 333398.5 | 2456903 | 1 | 420 |
| 352 | 235725.21 | 2095728 | 145 | 564 |
| 353 | 242472.14 | 4572916 | 1 | 509 |
| 354 | 253550.20 | 3397390 | 345 | 737 |
| 355 | 216262.3 | 3813934 | 1 | 555 |
| 356 | 235191.3 | 1997915 | 4 | 465 |
| 357 | 480337.45 | 4602215 | 509 | 796 |
| 358 | 199939.6 | 1858415 | 1177 | 1726 |
| 359 | 201204.9 | 4088394 | 1 | 634 |
| 360 | 201887.2 | 3478024 | 1 | 728 |
| 361 | 1136056.1 | 3527982 | 3200 | 3976 |
| 362 | 994977.1 | 3722056 | 1 | 272 |
| 363 | 888669.8 | 4721466 | 186 | 668 |
| 364 | 345860.20 | 5160686 | 734 | 1719 |
| 365 | 480337.43 | 1459082 | 321 | 851 |
| 365 | 480337.43 | 4602215 | 1 | 347 |
| 365 | 480337.43 | 1459082 | 321 | 851 |
| 365 | 480337.43 | 4602215 | 1 | 347 |
| 366 | 399300.14 | 2842978 | 1 | 444 |
| 367 | 368015.2 | 3115792 | 21 | 538 |
| 368 | 227550.1 | 3771020 | 1 | 1611 |
| 369 | 201906.6 | 3733666 | 439 | 850 |
| 370 | 349589.10 | 127321 | 165 | 543 |
| 371 | 3713867CB1 | 2061401 | 1852 | 2214 |
| 372 | 235943.27 | 2957567 | 1 | 717 |
| 373 | 241742.1 | 1295905 | 2642 | 2895 |
| 374 | 333680.1 | 3138456 | 2152 | 2344 |
| 375 | 411429.3 | 1424886 | 72 | 500 |
| 376 | 2356055CB1 | 2356055 | 52 | 994 |
| 377 | 239579.3 | 1704713 | 3029 | 3430 |
| 378 | 332240.1 | 2201507 | 2044 | 2648 |
| 379 | 255161.1 | 2270986 | 88 | 425 |
| 380 | 2454384CB1 | 2454384 | 195 | 763 |
| 381 | 1383298.3 | 1510539 | 309 | 789 |
| 382 | 383376.7 | 5551761 | 138 | 483 |
| 383 | 230816.1 | 1526322 | 345 | 1072 |
| 384 | 211949.2 | 1966295 | 1538 | 2202 |
| 385 | 238853.42 | 1450886 | 6 | 370 |
| 386 | 333165.2 | 1682337 | 2755 | 3521 |
| 387 | 344868.12 | 3333118 | 360 | 882 |
| 388 | 148304.14 | 3646303 | 281 | 814 |
| 389 | 247747.6 | 3124204 | 272 | 835 |
| 390 | 001153.12 | 5546984 | 1 | 398 |
| 391 | 1095728.19 | 414480 | 68 | 752 |
| 392 | 222604.7 | 2745735 | 502 | 776 |
| 393 | 1094984.14 | 2620487 | 0 | 544 |
| 394 | 400702.1 | 1969974 | 280 | 814 |
| 395 | 196557.1 | 1927026 | 442 | 874 |
| 396 | 237208.5 | 993365 | 1694 | 2020 |
| 397 | 337792.2 | 1832594 | 1229 | 1870 |
| 398 | 398970.3 | 4885619 | 1 | 497 |
| 399 | 1309633.3 | 1880421 | 388 | 662 |
| 400 | 981662.1 | 4401727 | 93 | 576 |
| 401 | 081187.1 | 2416447 | 1 | 64 |
| 402 | 086533.6 | 219839 | 954 | 1430 |
| 403 | 1093481.2 | 1962139 | 1177 | 1417 |
| 404 | 024494.7 | 1938744 | 5 | 461 |
| 405 | 888669.7 | 4721466 | 430 | 889 |
| 406 | 428206.1 | 2675284 | 877 | 1216 |
| 407 | 1328026.7 | 549728 | 2073 | 2667 |
| 408 | 475113.7 | 1700462 | 2797 | 3289 |
| 409 | 204392.5 | 2950394 | 236 | 615 |
| 410 | 1092257.12 | 3440567 | 1 | 532 |
| 411 | 1134990.3 | 3878116 | 751 | 1092 |
| 412 | 1095223.13 | 4158982 | 1 | 371 |
| 413 | 214654.1 | 1681727 | 1333 | 2088 |
| 414 | 086533.5 | 219839 | 325 | 900 |
| 415 | 474117.5 | 3992320 | −27 | 997 |
| 416 | 410812.1 | 3380665 | 0 | 573 |
| 417 | 011822.2 | 2887646 | 1 | 730 |
| 418 | 252899.7 | 2811710 | 1692 | 2837 |
| 419 | 1511488CB1 | 1511488 | 439 | 984 |
| 420 | 1383797.1 | 1466844 | 677 | 1059 |
| 421 | 400745.1 | 4205017 | 629 | 1348 |
| 422 | 029618.1 | 1223877 | 333 | 798 |
| 423 | 245722.8 | 2508079 | 19 | 1220 |
| 424 | 981488.1 | 2159213 | 51 | 951 |
| 424 | 981488.1 | 2449016 | 2332 | 2708 |
| 424 | 981488.1 | 2159213 | 51 | 951 |
| 424 | 981488.1 | 2449016 | 2332 | 2708 |
| 425 | 241858.1 | 4460483 | 1 | 392 |
| 426 | 1466276CB1 | 739191 | 1 | 1043 |
| 427 | 999386.3 | 5286390 | 340 | 1331 |
| 428 | 1091079.8 | 1503624 | 643 | 1329 |
| 429 | 407084.1 | 3423374 | 143 | 486 |
| 430 | 215642.2 | 1923709 | 2475 | 2828 |
| 431 | 1159769.1 | 3929017 | 27 | 826 |
| 432 | 482336.31 | 4104390 | 1467 | 2046 |
| 433 | 064703.1 | 1900111 | 1 | 555 |
| 434 | 294837.1 | 3255489 | 621 | 1064 |
| 435 | 982699.1 | 5394922 | 1 | 338 |
| 436 | 015126.1 | 4361169 | 94 | 349 |
| 437 | 235132.10 | 5682290 | 88 | 600 |
| 438 | 1070092.1 | 2965648 | −7 | 660 |
| 439 | 272599.1 | 2278772 | 1 | 278 |
| 440 | 415901.1 | 2998131 | −8 | 314 |
| 441 | 477387.3 | 3100048 | 1 | 439 |
| 442 | 208702.1 | 3658444 | 407 | 725 |
| 443 | 1135039.1 | 899118 | 2167 | 2457 |
| 444 | 232649.2 | 2234640 | 1483 | 2043 |
| 445 | 340450.1 | 4639995 | 1 | 225 |
| 446 | 320660.1 | 5034026 | 1 | 380 |
| 447 | 467104.21 | 2054055 | 2671 | 3274 |
| 448 | 332382.1 | 3999569 | 1 | 554 |
| 449 | 2104530CB1 | 2104530 | 139 | 463 |
| 450 | 007228.1 | 60306 | 635 | 846 |
| 451 | 008098.1 | 313312 | 49 | 739 |
| 452 | 016124.2 | 3732960 | 31 | 391 |
| 453 | 016149.1 | 3736058 | 1 | 363 |
| 454 | 025731.1 | 2586554 | 1093 | 1355 |
| 455 | 026856.1 | 1300730 | 1 | 711 |
| 456 | 028918.1 | 2800380 | 278 | 951 |
| 457 | 032481.1 | 4073339 | 937 | 1338 |
| 458 | 045630.1 | 1269876 | 1 | 462 |
| 459 | 047533.1 | 4276910 | 198 | 635 |
| 460 | 072540.1 | 2152929 | 165 | 691 |
| 461 | 076580.1 | 2264967 | 132 | 562 |
| 462 | 1072776.1 | 5505238 | 68 | 334 |
| 463 | 1100233.1 | 3725970 | 1 | 527 |
| 464 | 1102220.2 | 1299351 | 4246 | 4789 |
| 465 | 1328508.1 | 2669974 | 1215 | 1910 |
| 466 | 1383457.1 | 5024278 | 398 | 1014 |
| 467 | 140230.5 | 1211835 | 516 | 1070 |
| 468 | 150868.1 | 491129 | 208 | 467 |
| 469 | 172582.1 | 5622463 | 1 | 841 |
| 470 | 175536.1 | 122943 | 522 | 1216 |
| 470 | 175536.1 | 5187276 | 1238 | 2039 |
| 470 | 175536.1 | 122943 | 522 | 1216 |
| 470 | 175536.1 | 5187276 | 1238 | 2039 |
| 471 | 176928.1 | 2782090 | 1 | 389 |
| 472 | 197252.1 | 2656358 | 685 | 1675 |
| 473 | 1975480CB1 | 3852527 | 35 | 317 |
| 474 | 200578.1 | 473724 | 1662 | 2308 |
| 475 | 202117.4 | 1402988 | 593 | 1223 |
| 476 | 204750.3 | 2021485 | 17 | 401 |
| 477 | 205232.1 | 1379153 | 276 | 702 |
| 478 | 212157.1 | 3130003 | 132 | 610 |
| 479 | 221846.4 | 4116336 | 1 | 624 |
| 480 | 222092.1 | 2154847 | −96 | 381 |
| 481 | 228350.1 | 2605849 | 809 | 1435 |
| 482 | 229170.1 | 1818484 | 1 | 525 |
| 483 | 230820.1 | 3120378 | 1 | 617 |
| 484 | 231160.1 | 1848676 | 49 | 737 |
| 485 | 232212.1 | 1866738 | 39 | 415 |
| 486 | 234090.1 | 1718918 | 3061 | 3745 |
| 487 | 236432.1 | 3322032 | 400 | 1116 |
| 488 | 236497.1 | 1682609 | 128 | 781 |
| 489 | 237208.4 | 993365 | 12 | 552 |
| 490 | 2484813CB1 | 1338090 | 1953 | 2463 |
| 491 | 249104.1 | 2394947 | 1 | 657 |
| 492 | 287241.1 | 3412108 | 1 | 252 |
| 493 | 316571.1 | 5185101 | 1 | 632 |
| 494 | 331447.1 | 3363538 | 1406 | 1754 |
| 495 | 333127.1 | 153430 | 1 | 847 |
| 496 | 334025.5 | 3724351 | 85 | 469 |
| 497 | 334570.1 | 1296146 | 1 | 795 |
| 498 | 334814.1 | 1645745 | 1 | 367 |
| 499 | 335692.1 | 5023108 | 104 | 500 |
| 500 | 337407.1 | 3518117 | 284 | 748 |
| 501 | 337953.2 | 1378835 | 436 | 973 |
| 502 | 337953.5 | 1378835 | 1 | 261 |
| 503 | 346431.1 | 2723937 | 661 | 2265 |
| 504 | 368357.1 | 5085077 | 1 | 493 |
| 505 | 390546.1 | 5339122 | 1 | 282 |
| 506 | 400267.1 | 4240272 | 44 | 557 |
| 507 | 402288.1 | 3871646 | 304 | 728 |
| 508 | 403407.2 | 1316042 | 1 | 239 |
| 509 | 405447.1 | 3220727 | 207 | 528 |
| 510 | 407485.1 | 5552541 | 351 | 1226 |
| 511 | 427832.19 | 3692235 | 267 | 632 |
| 512 | 477913.1 | 2593092 | 1 | 85 |
| 513 | 902956.2 | 2607572 | 29 | 332 |
| 514 | 979367.4 | 2998372 | -112 | 170 |
| 515 | 979367.5 | 2998372 | 162 | 410 |
| 516 | 982861.1 | 3888854 | 36 | 545 |
| 517 | 983351.1 | 1819244 | 738 | 1922 |
| 518 | 984637.1 | 1610772 | 184 | 1164 |
| 519 | 984900.1 | 1802185 | 29 | 322 |

What is claimed is:

1. A combination comprising a plurality of cDNAs wherein the cDNAs are SEQ ID NOs:1-519 that are differentially expressed in respiratory disorders and the complements of SEQ ID NOs:1-519.

2. The combination of claim 1, wherein the cDNAs are SEQ ID NOs:1-12 that are differentially expressed at least 2.5-fold in respiratory disorders and the complements of SEQ ID NOs:1-12.

3. The combination of claim 1, wherein the cDNAs are SEQ ID NOs:13-83 that are differentially expressed at least 2.0-fold and the complements of SEQ ID NOs:13-83.

4. The combination of claim 1, wherein the cDNAs are SEQ ID NOs:98, 132, 135, 140, 145, 152, 174, 210, 223, 242, 246, 278, 304, 340, and 490 that are differentially expressed in squamous cell carcinoma thereby distinguishing squamous cell carcinoma from adenocarcinoma and the complements of SEQ ID NOs:98, 132, 135, 140, 145, 152, 174, 210, 223, 242, 246, 278, 304, 340, and 490.

5. The combination of claim 1, wherein the respiratory disorder is lung cancer, chronic obstructive pulmonary disease, emphysema, or asthma.

6. The combination of claim 1, wherein the cDNAs are immobilized on a substrate.

7. A method for detecting differential expression of one or more cDNAs in a sample containing nucleic acids, the method comprising:
a) hybridizing the substrate of claim 6 with nucleic acids of the sample, thereby forming one or more hybridization complexes;
b) detecting the hybridization complexes; and
c) comparing the hybridization complexes with those of a standard, wherein differences between the standard and sample hybridization complexes indicate differential expression of cDNAs in the sample.

8. The method of claim 7, wherein the nucleic acids of the sample are amplified prior to hybridization.

9. The method of claim 7, wherein the sample is from a subject with a respiratory disorder and comparison with a standard defines an early, mid, or late stage of that disorder.

10. A method of screening a plurality of molecules or compounds to identify a ligand which specifically binds a cDNA, the method comprising:
a) combining the combination of claim 1 with the plurality of molecules or compounds under conditions to allow specific binding; and
b) detecting specific binding between each cDNA and at least one molecule or compound, thereby identifying a ligand that specifically binds to each cDNA.

11. The method of claim 10 wherein the plurality of molecules or compounds are selected from DNA molecules, RNA molecules, peptide nucleic acid molecules, mimetics, peptides, transcription factors, repressors, and regulatory proteins.

12. An isolated cDNA comprising a nucleic acid sequence selected from SEQ ID NOs:12, 45, 51, 56, 64, 70, 72, 75-83, 344, 346, 375, 376, 377, 402, 406, 407, 412, 419, and 431.

13. A vector containing the cDNA of claim 12.

14. A host cell containing the vector of claim 13.

15. A method for producing a protein, the method comprising the steps of:
a) culturing the host cell of claim 14 under conditions for expression of protein; and
b) recovering the protein from the host cell culture.

16. A protein or a portion thereof produced by the method of claim 15.

17. A method for using a protein to screen a plurality of molecules or compounds to identify at least one ligand which specifically binds the protein, the method comprising:
a) combining the protein of claim 16 with the plurality of molecules or compounds under conditions to allow specific binding; and
b) detecting specific binding between the protein and a molecule or compound, thereby identifying a ligand which specifically binds the protein.

18. The method of claim 17 wherein the plurality of molecules or compounds is selected from agonists, antagonists, antibodies, DNA molecules, small molecule drugs, immunoglobulins, inhibitors, mimetics, peptide nucleic acids, peptides, pharmaceutical agents, proteins, RNA molecules, and ribozymes.

19. An antagonist identified by the method of claim 16.

20. An isolated antibody which specifically binds to a protein of claim 16.

21. The antibody of claim 20, wherein the antibody is selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

22. A method of using a protein to prepare and purify a polyclonal antibody comprising:
a) immunizing a animal with a protein of claim 16 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

23. A polyclonal antibody produced by the method of claim 22.

24. A method of using a protein to prepare a monoclonal antibody comprising:
a) immunizing a animal with a protein of claim 16 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies from culture.

25. A monoclonal antibody produced by the method of claim 24.

26. A method for using an antibody to detect expression of a protein in a sample, the method comprising:
a) combining the antibody of claim 20 with a sample under conditions which allow the formation of antibody:protein complexes; and
b) detecting complex formation, wherein complex formation indicates expression of the protein in the sample.

27. The method of claim 26 wherein complex formation is compared with standards and is diagnostic of a lung cancer.

28. A composition comprising an antibody of claim 20 and a labeling moiety or a pharmaceutical agent..
